(19) United States
(12) Patent Application Publication
(10) Pub. No.: US 2025/0002887 A1
(43) Pub. Date: Jan. 2, 2025

(54) ENGINEERED ACID ALPHA-GLUCOSIDASE VARIANTS

(71) Applicant: Crosswalk Therapeutics, Inc., Cambridge, MA (US)

(72) Inventors: Rachel Cathleen Botham, Burlingame, CA (US); Gordon Michael Cheung, San Jose, CA (US); William Casey Hallows, San Francisco, CA (US); Gjalt W. Huisman, Redwood City, CA (US); Nikki D. Kruse, San Carlos, CA (US); Jessica P. Lao, Emeryville, CA (US); Shilpa Maroju, Union City, CA (US); Adam P. Silverman, San Carlos, CA (US)

(21) Appl. No.: 18/759,313

(22) Filed: Jun. 28, 2024

Related U.S. Application Data
(60) Provisional application No. 63/511,347, filed on Jun. 30, 2023.

(51) Int. Cl. C12N 9/24; A61K 38/47; C12N 15/85

(52) U.S. Cl. CPC C12N 9/2402; A61K 38/47; C12N 15/85; C12Y 302/0102

(57) ABSTRACT

The present disclosure provides engineered acid alpha-glucosidase polypeptides, recombinant polynucleotides encoding the engineered acid alpha-glucosidase polypeptides, and method of using the engineered acid alpha-glucosidase polypeptides and the recombinant polynucleotides for therapeutic purposes.

### CROSS-REFERENCE TO RELATED APPLICATIONS

This application claims the benefit of U.S. Provisional Application No. 63/511,347, filed Jun. 30, 2023, which is incorporated by reference herein in its entirety.

### REFERENCE TO SEQUENCE LISTING, TABLE OR COMPUTER PROGRAM

The Sequence Listing concurrently submitted herewith as a file name CX7-223US1_ST26.xml, created on Jun. 28, 2024, with a file size of 2,391,488 bytes, is part of the specification and is incorporated by reference herein.

### TECHNICAL FIELD

The present disclosure relates to engineered acid alpha-glucosidase (GAA) polypeptides, compositions thereof, polynucleotides encoding the engineered acid alpha-glucosidase polypeptides, and uses of the engineered polypeptides for therapeutic and other purposes.

### BACKGROUND

Pompe disease is an autosomal recessive lysosomal storage disorder caused by mutations in the gene encoding acid alpha-glucosidase (GAA). This genetic defect leads to reduction in or absence of acid alpha-glucosidase in the body tissues. The resulting accumulation of glycogen in the lysosomes results in lysosomal swelling and rupture, which can lead to cell damage, organelle dysfunction, and other cellular defects. There are two primary forms of Pompe disease, including the classical infantile form and late-onset (childhood or adulthood) form, with some patients displaying an intermediate phenotype. Disease severity is related to the amount of enzyme activity present in the cells of affected individuals. The infantile form is the most severe and a rapidly progressive form, typically with acid alpha-glucosidase activity that is less than 1%, resulting in marked accumulation of glycogen in skeletal muscle as well as heart and other tissues (see, e.g., Hahn and Schanzer, Ann. Transl. Med., 2019, 7:283). In these patients, there is multi-system storage of accumulated lysosomal and non-lysosomal bound-glycogen in the heart, skeletal muscle, and brain tissue (see, e.g., Schoser, Ann. Transl. Med., 2019, 7:292). Patients have elevated creatine kinase levels, hypertrophic cardiomyopathy, failure to thrive, muscular hypotonia, and axial muscle weakness. If untreated, patients typically die within the first year of life due to cardiorespiratory insufficiency. Survival beyond 18 months of age is exceptional.

The infantile form is distinguished from non-classic or late-infantile Pompe disease in which patients display much less severe cardiac hypertrophy. Patients with late-onset Pompe disease typically experience progressive limb-girdle myopathy and respiratory dysfunction. These patients present with predominant, but not exclusive, muscle involvement. The patients eventually become wheelchair and/or ventilator-dependent. Respiratory insufficiency is the leading cause of death in these patients. Some patients may synthesize a non-functional form of acid alpha-glucosidase, but others do not produce any cross-reactive immunologic material of the native enzyme.

The human gene encoding acid alpha-glucosidase has been localized to chromosome 17q25.2-q25.3 and has been cloned and sequenced (see, e.g., Peruzzo et al., Ann. Transl. Med., 2019, 7:278-287; and Martiniuk et al., DNA Cell. Biol., 1991, 10:283-292). Although numerous mutations in the gene have been reported, the pathological mechanisms that lead to the wide range of phenotypes observed in affected patients remain unknown. Despite the availability of enzyme replacement therapy (ERT) utilizing recombinant human acid alpha-glucosidase, there remains the need for better treatment and management options for affected patients.

### SUMMARY

The present disclosure provides acid alpha-glucosidase polypeptides engineered to have improved properties, particularly as compared to the naturally occurring human acid alpha-glucosidase.

In some embodiments, the present disclosure provides an engineered acid alpha-glucosidase polypeptide, or biologically active fragment thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-944 of an even numbered SEQ ID NO. of SEQ ID NOs: 2, 12, and 14-754, or to an even numbered SEQ ID NO. of SEQ ID NOs: 2, 12, and 14-754, wherein the amino acid sequence comprises one or more substitutions relative to the reference sequence corresponding to residues 20-944 of SEQ ID NO: 12 or 2, or relative to the reference sequence corresponding to SEQ ID NO: 12 or 2.

In some embodiments, the engineered acid alpha-glucosidase, or biologically active fragment thereof, comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20 to 944 of SEQ ID NO: 12 or 2, or to a reference sequence corresponding to SEQ ID NO: 12 or 2, wherein the amino acid sequence comprises one or more substitutions relative to the reference sequence corresponding to residues 20 to 944 of SEQ ID NO: 12 or 2, or relative to the reference sequence corresponding to SEQ ID NO: 12 or 2.

In some embodiments, the engineered acid alpha-glucosidase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-944 of an even numbered SEQ ID NO. of SEQ ID NOs: 14-754, or to an even numbered SEQ ID NO. of SEQ ID NOs: 14-754, wherein the amino acid sequence comprises one or more substitutions relative to the reference sequence corresponding to residues 20-944 of SEQ ID NO: 12 or 2, or relative to the reference sequence corresponding to SEQ ID NO: 12 or 2.

In some embodiments, the amino acid sequence of the engineered acid alpha-glucosidase comprises at least a substitution at amino acid position 24, 28, 29, 39, 50, 62, 78, 87, 135, 150, 266, 267, 305, 437, 486, 522, 569, 670, 692, 711, 736, 750, 812, 830, 842, 871, 883, 894, 913, or 932, or combinations thereof, wherein the amino acid positions are relative to the reference sequence corresponding to residues 20-944 of SEQ ID NO: 12, or relative to the reference sequence corresponding to SEQ ID NO: 12.

In some embodiments, the amino acid sequence of the engineered acid alpha-glucosidase comprises at least a substitution 24A/C/D/E/F/G/H/I/K/L/M/N/P/R/S/T/V/Y, 28A/C/D/E/F/G/H/K/L/P/Q/R/T/V/W, 29A/C/D/E/F/G/H/I/K/L/M/N/P/Q/R/S/V/W/Y, 39A/E/F/G/I/L/N/T, 50A/C/D/E/F/G/H/I/K/L/M/N/Q/R/S/T/W/Y, 62A/D/E/F/G/H/I/K/M/N/P/Q/S/T/V/Y, 78A/C/D/F/G/H/I/K/L/M/N/P/Q/R/S/T/V/W/Y, 87A/D/G/H/I/K/L/MN/Q/R/S/T/V/W, 135A/C/D/E/F/G/H/I/K/L/N/P/R/Y, 150T, 266A/D/E/H/K/Q/T, 267H/L/R/T/V, 305V, 437A/H/S, 486A/C/D/F/G/H/I/K/L/M/N/Q/R/S/T/V/W/Y, 522A/C/D/E/F/G/H/I/K/L/M/N/P/Q/R/S/T/W/Y, 569A/C/D/E/G/H/I/K/L/M/N/P/Q/R/S/V/W/Y, 670A/D/E/F/G/H/I/K/L/M/N/Q/R/S/V/Y, 692A/C/D/E/F/H/I/K/L/M/N/Q/R/S/T/V/W/Y, 711A/C/D/E/F/G/I/K/L/M/N/Q/R/S/T/V/W/Y, 736F/L, 750A/E/K/L/Q/R, 812A/D/G/S, 830D/E/F/G/H/L/M/N/Q/S/T/V/W/Y, 842A/C/D/E/F/G/H/K/L/M/N/Q/R/T/W, 871A/C/D/F/G/H/I/K/L/M/N/Q/R/S/T/V/W/Y, 883A/F/Q, 894A/C/D/E/H/I/K/L/M/N/Q/R/S/T/V/W/Y, 913E/F/H/I/K/M/N/Q/S/W, or 932C/D/E/G/H/K/L/M/N/P/Q/R/S/T/W/Y, or combinations thereof, wherein the amino acid positions are relative to the reference sequence corresponding to residues 20-944 of SEQ ID NO: 12, or relative to the reference sequence corresponding to SEQ ID NO: 12.

In some embodiments, the amino acid sequence of the engineered acid alpha-glucosidase comprises at least a substitution 24A/C/D/F/G/H/I/K/M/N/P/S/T/V/Y, 28A/C/D/E/F/G/H/K/Q/T/V/W, 29A/C/D/E/F/G/H/I/K/M/N/P/R/W/Y, 39A/E/F/G/I/L/N/T, 50A/C/D/E/F/H/I/K/M/N/R/S/T/W/Y, 62D/H/I/K/M/N/P/Q/Y, 78A/C/D/F/G/H/I/K/L/M/N/Q/R/S/T/V/W/Y, 87A/G/H/I/K/L/MN/Q/R/S/T/V/W, 135C/D/E/F/G/H/I/K/L/N/R/Y, 266A/D/E/H/K/Q, 267H/L/T/V, 305V, 437A/H, 486C/D/F/G/H/I/K/L/M/N/Q/R/S/V/W/Y, 522A/C/D/F/G/H/I/K/L/M/N/P/Q/R/S/T/W/Y, 569A/C/D/E/G/K/M/N/P/R/W, 670A/D/G/H/K/M/Y, 692A/D/E/H/K/L/M/N/T/W, 711D/E/I/K/M/N/Q/S/T/V/Y, 736F/L, 750E/K/L/Q/R, 812A/D/G/S, 830D/E/F/G/H/L/M/N/S/T/W/Y, 842A/C/D/F/H/K/L/M/N/Q/R/T/W, 871A/C/D/F/H/I/M/N/Q/T/V/W/Y, 883A/F/Q, 894A/D/E/H/I/K/L/M/N/S/T/V/W/Y, 913F/IK/M/N/S, or 932C/D/E/G/H/K/L/M/N/P/Q/R/W/Y, or combinations thereof, wherein the amino acid positions are relative to the reference sequence corresponding to residues 20-944 of SEQ ID NO: 12, or relative to the reference sequence corresponding to SEQ ID NO: 12.

In some embodiments, the amino acid sequence of the engineered acid alpha-glucosidase comprises a sequence comprising residues 20 to 944 of an even-numbered SEQ ID NO. of SEQ ID NOs: 14-754, or a sequence comprising an even-numbered SEQ ID NO. of SEQ ID NOs: 14-754.

In some embodiments, the amino acid sequence of the engineered acid alpha-glucosidase comprises a sequence comprising residues 20 to 944 of SEQ ID NO: 14, 114, 126, 170, 250, 252, 394, 472, 488, or 506, or a sequence comprising SEQ ID NO: 14, 114, 126, 170, 250, 252, 394, 472, 488, or 506.

In some embodiments, the engineered acid alpha-glucosidase exhibits at least one improved property compared to a reference acid alpha-glucosidase. In some embodiments, engineered acid alpha-glucosidase exhibits at least one improved property selected from: i) enhanced catalytic activity; ii) increased tolerance to pH 7; iii) increased tolerance to pH 4.4; iv) increased stability in lysosomes; v) increased expression in cells; vi) increased uptake into cells; vii) increased enzymatic activity in cell lysates; viii) increased stability in plasma/serum; and ix) reduced immunogenicity; or a combination of any of i), ii), iii), iv), v), vi), vii), viii), and ix), as compared to a reference acid alpha-glucosidase having a sequence corresponding to residues 20 to 944 of SEQ ID NO: 2 or 12, or a sequence corresponding to SEQ ID NO: 2 or 12.

In a further aspect, the present disclosure provides a recombinant polynucleotide comprising a polynucleotide sequence encoding an engineered acid alpha-glucosidase disclosed herein.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference polynucleotide sequence corresponding to nucleotide residues 58-2832 of an odd-numbered SEQ ID NO. of SEQ ID NOs: 13-753, or to a reference polynucleotide sequence corresponding to an odd-numbered SEQ ID NO. of SEQ ID NOs: 13-753, wherein the polynucleotide encodes an acid alpha-glucosidase.

In some embodiments, the recombinant polynucleotide encoding an engineered acid alpha-glucosidase comprises a polynucleotide sequence codon optimized for expression of the engineered acid alpha-glucosidase.

In some embodiments, the recombinant polynucleotide comprises a polynucleotide sequence comprising nucleotide residues 58-2832 of an odd-numbered SEQ ID NO. of SEQ ID NOs: 13-753, or a polynucleotide sequence comprising an odd-numbered SEQ ID NO. of SEQ ID NOs: 13-753.

In a further aspect, the present disclosure provides an expression vector comprising a recombinant polynucleotide encoding an engineered acid alpha-glucosidase described herein. In some embodiments, the expression vector comprises a control sequence operably linked to a recombinant polynucleotide encoding an engineered acid alpha-glucosidase. In some embodiments, the control sequence is a promoter, such as a heterologous promoter.

In another aspect, provide herein are host cells comprising an expression vector comprising the recombinant polynucleotide described herein. In some embodiments, the host cell is a eukaryotic or prokaryotic cell. In some embodiments, the host cell is a mammalian cell, particularly a human cell. In some embodiments, the human cell is from a patient having a deficiency in acid alpha-glucosidase activity, for example a patient afflicted with Pompe disease.

In another aspect, the host cells are used to produce an engineered acid alpha-glucosidase disclosed herein. In some embodiments, a method of producing an engineered acid alpha-glucosidase, comprising culturing the host cell comprising an expression vector under suitable conditions such that the engineered acid alpha-glucosidase is produced.

In another aspect, the present disclosure provides a pharmaceutical composition comprising an engineered acid alpha-glucosidase or a recombinant polynucleotide encoding the engineered acid alpha-glucosidase, including an expression vector comprising the recombinant polynucleotide. In some embodiments, the pharmaceutical composition further comprises a pharmaceutically acceptable carrier and/or excipient.

In another aspect, the engineered acid alpha-glucosidase or the recombinant polynucleotide encoding the engineered acid alpha-glucosidase is used for treating a subject with a deficiency in acid alpha-glucosidase activity. In some embodiments, a method for treating and/or preventing symptoms of a deficiency in acid alpha-glucosidase in a subject, comprises administering to a subject in need thereof an effective amount of an engineered acid alpha-glucosidase or a recombinant polynucleotide encoding the engineered acid alpha-glucosidase disclosed herein.

In some embodiments, a pharmaceutical composition comprising the engineered acid alpha-glucosidase or the recombinant polynucleotide encoding the engineered acid alpha-glucosidase is administered to a subject.

In some embodiments, the subject for treatment is afflicted with Pompe disease. In some embodiments, the subject is an infant or child. In some embodiments, the subject is an adult or young adult.

In a further aspect, the present disclosure provides for use of an engineered acid alpha-glucosidase or a recombinant polynucleotide encoding the engineered acid alpha-glucosidase, or a pharmaceutical composition thereof for treating a deficiency in acid alpha-glucosidase activity. In some embodiments, the deficiency in acid alpha-glucosidase is Pompe disease.

### BRIEF DESCRIPTION OF THE DRAWINGS

FIG. 1 provides a graph showing the duration of stability of six GAA variants in neutral pH cell culture medium, as described in Example 6.

FIG. 2 provides a graph showing the melting temperature of six GAA variants at lysosomal (4.4) and slightly basic (7.4) pH, as described in Example 6.

FIG. 3 provides a graph showing the duration of stability of six GAA variants when challenged by treatment with plasma, as described in Example 6.

FIG. 4 provides graphs showing the 4-MU-GLU hydrolysis activity in lysates from cultured Pompe patient derived fibroblasts treated with seven purified GAA variants for durations of 1 hour (Panel 4A), 4 hours (Panel 4B), 24 hours (Panel 4C), or 96 hours (Panel 4D), wash-out of GAA material, and incubation at 37° C. until 96 hours post treatment, as described in Example 6. Values are expressed as RFU activity.

FIG. 5 provides graphs showing the 4-MU-GLU hydrolysis activity in lysates from cultured C2C12 GAA knockout myoblasts treated with seven purified GAA variants for durations of 1 hour (Panel 5A), 4 hours (Panel 5B), 24 hours (Panel 5C), or 96 hours (Panel 5D), wash-out of GAA material, and incubation at 37° C. until 96 hr post treatment, as described in Example 6. Values are expressed as RFU activity.

FIG. 6 provides a graph showing the 4-MU-GLU hydrolysis activity in the supernatant of cultures of C2C12 GAA knockout myoblasts that were transfected with plasmid DNA of six GAA variants.

FIG. 7 provides graphs showing the GAA activity (4-MU-GLU hydrolysis shown in Panel 7A, and glycogen hydrolysis shown in Panel 7B) in normalized lysates of C2C12 GAA knockout myoblasts that were transfected with plasmid DNA of six GAA variants.

FIG. 8 shows in Panel 8A the sum of all high quality GAA-derived peptides that were observed by mass spectrometry from each GAA variant in an ex vivo MHC II associated peptide proteomics assay (MAPPs assay), and in Panel 8B the peptide frequency across donors plotted against the region of the GAA sequence from which the peptide was processed from for each GAA variant. In the MAPPs assay, PBMCs from healthy donors were differentiated into dendritic cells (antigen presenting cells) and incubated in the presence of GAA variants (the antigen). HLA-binding peptides are then eluted from HLA-DR molecules and identified by mass spectrometry, providing information on the processing and presentation of peptides in antigen presenting cells. These results indicate that GAA variants of SEQ ID NO: 6, SEQ ID NO: 8, and SEQ ID NO: 14 have significantly reduced processing and reduced frequency of peptide presentation as compared to the WT GAA, SEQ ID NO: 2.

### DETAILED DESCRIPTION

The present disclosure provides engineered acid alpha-glucosidase (GAA) polypeptides and compositions thereof. In some embodiments, the engineered acid alpha-glucosidase polypeptides are engineered to display improved properties, including enhanced catalytic activity and enhanced acid stability, while reducing sensitivity to proteolysis. The present disclosure also provides methods for using the engineered acid alpha-glucosidase polypeptides, including compositions thereof, for therapeutic and other purposes.

### Abbreviations and Definitions

In reference to the present disclosure, the technical and scientific terms used in the descriptions herein will have the meanings commonly understood by one of ordinary skill in the art, unless specifically defined otherwise.

It is to be understood that the invention described herein is not limited to the particular methodology, protocols, and reagents described, as these may vary, depending upon the context they are used by those of skill in the art. Accordingly, the terms defined immediately below are more fully described by reference to the application as a whole.

Furthermore, the section headings provided herein are not to be construed as limitations of the various aspects or embodiments of the invention which can be had by reference to the application as a whole.

As used herein, the singular “a”, “an,” and “the” include the plural references, unless the context clearly indicates otherwise.

As used herein, the term “comprising” and its cognates are used in their inclusive sense (i.e., equivalent to the term “including” and its corresponding cognates).

It is also to be understood that where description of embodiments use the term “comprising” and its cognates, the embodiments can also be described using language “consisting essentially of” or “consisting of.”

Moreover, numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

“About” means an acceptable error for a particular value. In some instances, “about” means within 0.05%, 0.5%, 1.0%, or 2.0%, of a given value range. In some instances, “about” means within 1, 2, 3, or 4 standard deviations of a given value. In some instances, “about” encompasses values that are within 2.5%, 3%, 3.5%, 4%, 4.5%, 5%, 5.5%, 6%, 6.5%, 7%, 7.5%, 8%, 8.5%, 9%, 9.5%, or 10% of a given value.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably herein to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glycine (Gly or G), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (Cα). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“Acid alpha-glucosidase,” “acid α-glucosidase,” “acid alpha-glucosidase polypeptide” “lysosomal alpha-glucosidase,” and “GAA” refer to enzymes within a family (EC 3.2.1.20) of enzymes that break down glycogen present in lysosomes. The enzyme is also sometimes referred to as “alpha-1,4-glucosidase,” α-1,4-glucosidase,” “acid maltase,” “glucoinvertase,” “glucosidosucrase,” “lysosomal alpha-glucosidase,” “lysosomal α-glucosidase,” “maltase,” or “maltase-glucoamylase.” One reaction catalyzed by the enzyme is the hydrolysis of terminal, non-reducing (1 to 4) linked alpha-D-glucose residues with release of alpha-D-glucose. As used herein, the term “rhGAA” refers to recombinant human acid alpha-glucosidase.

“Pompe disease” refers to a glycogen storage disease type II, which is typically an autosomal recessive genetic disorder that results in a metabolic disorder characterized by lysosomal accumulation of glycogen in skeletal muscle and other tissues. It is characterized based on age of onset, organ involvement, severity, and rate of progression. The more severe form is infantile-onset Pompe disease (IOPD), which occurs in infants. The other form, referred to as “late-onset Pompe disease” (LOPD), occurs in individuals with an onset of disease before 12 months of age, but without the cardiomyopathy associated with IOPD, and all individuals with an onset of disease after 12 months of age. Synonyms for Pompe disease include “acid alpha-glucosidase deficiency,” “acid maltase deficiency,” “GAA deficiency,” “glycogen storage disease type II,” “GSD II,” “GSD2,” and “glycogenosis type II.”

“Polynucleotide,” “nucleic acid,” or “oligonucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When a polynucleotide, nucleic acid, or oligonucleotide sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid. The polynucleotide or nucleic acid may be single-stranded or double-stranded, or may include both single-stranded regions and double-stranded regions.

“Engineered,” “recombinant,” “non-naturally occurring,” and “variant,” when used with reference to a cell, a polynucleotide or a polypeptide refers to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation. In some embodiments, “wild-type” and “naturally-occurring” refer to the form found in nature that has normal function and/or activity.

“Coding sequence” refers to that part of a nucleic acid (e.g., a gene) that encodes an amino acid sequence of a protein.

“Percent (%) sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol., 1990, 215:403-410; and Altschul et al., Nucleic Acids Res., 1997, 25(17):3389-3402, respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (See, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See, Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered acid alpha-glucosidase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence.

“Amino acid difference” or “residue difference” refers to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X24 as compared to SEQ ID NO: 12” refers to a difference of the amino acid residue at the polypeptide position corresponding to position 24 of SEQ ID NO: 12. Thus, if the reference polypeptide of SEQ ID NO: 12 has a tryptophan at position 24, then a “residue difference at position X24 as compared to SEQ ID NO: 2” an amino acid substitution of any residue other than tryptophan at the position of the polypeptide corresponding to position 24 of SEQ ID NO: 12. In most instances herein, the specific amino acid residue difference at a position is indicated as “XnY” where “Xn” specifies the corresponding position as described above, and “Y” is the single letter identifier of the amino acid found in the engineered polypeptide (i.e., the different residue than in the reference polypeptide). In some instances (e.g., as shown in in Table 3-1), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some embodiments, the amino acid difference, e.g., a substitution, is denoted by the abbreviation “nB,” without the identifier for the residue in the reference sequence. In some embodiments, the phrase “an amino acid residue nB” denotes the presence of the amino residue in the engineered polypeptide, which may or may not be a substitution in context of a reference sequence.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X24A/X24C or X24A/C or 24A/C). In some embodiments, the amino acid residue is selected from the various alternative amino acid residues listed for that residue position. In some embodiments, the polypeptide variants comprise more than one substitution. These substitutions are separated by a slash for ease in reading (e.g., L24W/L28S or 24W/28S) or by a semicolon, as noted below. In some cases, as noted in the foregoing, the “X” does not precede the position number in the present application.

“Amino acid substitution set” and “substitution set” refers to a group of amino acid substitutions within a polypeptide sequence. In some embodiments, substitution set comprises 2, 3, 4, 5, 6, 7, 8, 9, 10, 11, 12, 13, 14, 15, or more amino acid substitutions. In some embodiments, a substitution set refers to the set of amino acid substitutions that is present in any of the acid alpha-glucosidase polypeptides listed in any of the Tables in the Examples. In these substitution sets, the individual substitutions are separated by a semicolon (e.g., L24W; L28S) or slash (“/”; e.g., L24W/L28S or 24W/28S). In some embodiments, the “substitution” comprises the deletion of an amino acid, and can be denoted by “−” symbol.

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide while retaining activity and/or retaining the improved properties of an engineered polypeptide. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” or a “biologically active fragment” used interchangeably herein refers to a polypeptide that has an amino-terminal and/or carboxy-terminal deletion(s) and/or internal deletions, but where the remaining amino acid sequence is identical to the corresponding positions in the sequence to which it is being compared (e.g., a full-length engineered acid alpha-glucosidase of the present invention) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The engineered acid alpha-glucosidase polypeptides may be present within a cell, present in the cellular medium, or prepared in various forms, such as lysates or isolated preparations. As such, in some embodiments, the engineered acid alpha-glucosidase polypeptides can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure polypeptide composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated polypeptides are substantially pure polypeptide compositions.

“Improved enzyme property” refers to an engineered acid alpha-glucosidase polypeptide that exhibits an improvement in any enzyme property as compared to a reference acid alpha-glucosidase polypeptide, which can be a wild-type acid alpha-glucosidase polypeptide or another engineered acid alpha-glucosidase polypeptide. Improved properties include but are not limited to such properties as increased protein expression, increased thermoactivity, increased thermostability, increased pH activity, increased stability, increased enzymatic activity, increased specific activity, increased resistance to substrate or end-product inhibition, increased chemical stability, improved solvent stability, increased tolerance to acidic, neutral, or basic pH, increased tolerance to proteolytic activity (i.e., reduced sensitivity to proteolysis), reduced aggregation, increased solubility, reduced immunogenicity, improved post-translational modification (e.g., glycosylation), altered temperature profile, increased lysosomal stability, etc.

“Increased enzymatic activity” or “enhanced catalytic activity” refers to an improved property of the engineered acid alpha-glucosidase polypeptides, which can be represented by an increase in specific activity (e.g., product produced/time/weight protein) or an increase in percent conversion of the substrate to the product (e.g., percent conversion of starting amount of substrate to product in a specified time period using a specified amount of acid alpha-glucosidase) as compared to the reference acid alpha-glucosidase enzyme. Exemplary methods to determine enzyme activity are provided in the Examples.

Any property relating to enzyme activity may be affected, including the classical enzyme properties of Kₘ, Vₘₐₓ or k_{cat}, changes of which can lead to increased enzymatic activity. Improvements in enzyme activity can be from about 1.1 fold the enzymatic activity of the corresponding wild-type enzyme, to as much as 2-fold, 5-fold, 10-fold, 20-fold, 25-fold, 50-fold, 75-fold, 100-fold, 150-fold, 200-fold or more enzymatic activity than the naturally occurring acid alpha-glucosidase or another engineered acid alpha-glucosidase from which the acid alpha-glucosidase polypeptides were derived.

In some embodiments, acid alpha-glucosidase activity can be measured by any suitable method known in the art (e.g., standard assays, such as monitoring changes in spectrophotometric properties of reactants or products). In some embodiments, the amount of product produced can be measured by monitoring fluorescence (Ex. 355 nm, Em. 460 nm) after hydrolysis of a 4-methylumbelliferyl-alpha-D-glucopyranoside (4-MUGlu) molecule, such as provided in the Examples. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

“Improved tolerance to acidic pH” means that an engineered acid alpha-glucosidase according to the invention will have increased stability (e.g., higher retained activity at about pH 4-4.8, after exposure to an acidic pH for a specified period of time, e.g., 1 hour, up to 24 hours) as compared to a reference acid alpha-glucosidase or another enzyme.

“Improved tolerance to neutral pH” means that an engineered acid alpha-glucosidase according to the invention will have increased stability (higher retained activity at about pH 7, after exposure to a neutral pH for a specified period of time, e.g., 1 hour, and up to 24 hours) as compared to a reference acid alpha-glucosidase or another enzyme.

“Improved cellular uptake” means that an engineered acid alpha-glucosidase provided herein exhibits increased endocytosis into cells, as compared to a reference acid alpha-glucosidase (including wild-type acid alpha-glucosidase) or another enzyme. In some embodiments, the cells are cultured Pompe patient-derived cells (higher retained intracellular activity after incubation with cultured cells over a specified period of time, as compared to a reference acid alpha-glucosidase or another enzyme). In some additional embodiments, the engineered acid alpha-glucosidase provided herein exhibits greater retained intracellular activity with cultured cells over a specific period of time as compared to a reference acid alpha-glucosidase (including wild-type acid alpha-glucosidase) or another enzyme. In some additional embodiments, the time period is about 4 hours, while in some other embodiments, the time period is less than 4 hours (e.g., 1, 2, or 3 hours), and in some alternative embodiments, the time period is more than 4 hours (e.g., 5, 6, 7, 8, or more hours).

“Reduced immunogenicity” and “decreased immunogenicity” mean that an engineered acid alpha-glucosidase provided herein induces or is predicted to have a reduced immune response as compared to a wild-type or another reference acid alpha-glucosidase.

“Physiological pH” as used herein means the pH range generally found in a subject's (e.g., human) blood.

“Neutral pH” (e.g., used with reference to improved stability to basic pH conditions or increased tolerance to basic pH) means a pH of about 7.

“Basic pH” (e.g., used with reference to improved stability to basic pH conditions or increased tolerance to basic pH) means a pH >7, e.g., in a pH range of >7 to 11.

“Acidic pH” (e.g., used with reference to improved stability to acidic pH conditions or increased tolerance to acidic pH) means a pH<7, e.g., in a pH range of about 1.5 to 4.8.

“Conversion” refers to the enzymatic conversion (or biotransformation) of a substrate(s) to the corresponding product(s). “Percent conversion” refers to the percent of the substrate that is converted to the product within a period of time under specified conditions. Thus, the “enzymatic activity” or “activity” of an acid alpha-glucosidase polypeptide can be expressed as “percent conversion” of the substrate to the product in a specific period of time.

“Suitable reaction conditions” refers to those conditions in the enzymatic conversion reaction solution (e.g., ranges of enzyme loading, substrate loading, temperature, pH, buffers, co-solvents, etc.) under which an acid alpha-glucosidase polypeptide of the present application is capable of converting a substrate to the desired product compound. Exemplary “suitable reaction conditions” are provided in the present application and illustrated by the Examples. “Loading”, such as in “compound loading” or “enzyme loading” refers to the concentration or amount of a component in a reaction mixture at the start of the reaction. “Substrate” in the context of an enzymatic conversion reaction process refers to the compound or molecule acted on by the acid alpha-glucosidase polypeptide. “Product” in the context of an enzymatic conversion process refers to the compound or molecule resulting from the action of the acid alpha-glucosidase polypeptide on a substrate.

“Codon optimized” refers to changes in the codons of the polynucleotide encoding a protein to those preferentially used in a particular organism such that the encoded protein is more efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the acid alpha-glucosidase enzymes may be codon optimized for optimal production from the host organism selected for expression.

“Control sequence” refers herein to include all components, which are necessary or advantageous for the expression of a polynucleotide and/or polypeptide of the present application. Each control sequence may be native or foreign to the nucleic acid sequence encoding the polypeptide. Such control sequences include, but are not limited to, a leader, polyadenylation sequence, propeptide sequence, promoter sequence, signal peptide sequence, initiation sequence and transcription terminator. In some embodiments, at a minimum the control sequences include a promoter, and transcriptional and translational stop signals.

“Operably linked” or “operatively linked” is defined herein as a configuration in which a control sequence is appropriately placed (i.e., in a functional relationship) at a position relative to a polynucleotide of interest such that the control sequence directs or regulates the expression of the polynucleotide, and where appropriate an encoded polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Vector” refers to a polynucleotide construct for introducing a polynucleotide sequence into a cell. In some embodiments, the vector is an “expression vector” that is operably linked to a suitable control sequence capable of effecting the expression in a suitable host of a polynucleotide of interest, and/or a polypeptide encoded in the polynucleotide. In some embodiments, an expression vector has a promoter sequence operably linked to the polynucleotide sequence (e.g., transgene) to drive expression in a host cell, and in some embodiments, also comprises a transcription terminator sequence.

“Expression” includes any step involved in the production of the polypeptide including, but not limited to, transcription, post-transcriptional modification, translation, and post-translational modification. In some embodiments, the term also encompasses secretion of the polypeptide from a cell.

“Culturing” refers to the growing of a population of cells under any suitable conditions (e.g., using a liquid, gel or solid medium).

“Produces” refers to the production or expression of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or protein sequences (e.g., a promoter sequence, signal peptide, terminator sequence, etc.) that are derived from different sources and are not associated in nature.

“Host cell” and “host strain” refer to suitable hosts for expression vectors comprising polynucleotides provided herein (e.g., the polynucleotides encoding the acid alpha-glucosidase variants).

In some embodiments, the host cells are prokaryotic or eukaryotic cells that have been transformed or transfected with vectors constructed using recombinant DNA techniques as known in the art.

“Therapeutic” refers to a compound administered to a subject who shows signs or symptoms of pathology having beneficial or desirable medical effects.

“Gene therapy vector” refers to a vehicle or carrier suitable for delivery of a polynucleotide to cells for therapeutic effect. In some embodiments, the vector encapsulates a gene (e.g., therapeutic genes) or polynucleotide sequences for delivery to cells or tissues, including but not limited to adenovirus (AV), adeno-associated virus (AAV), lentivirus (LV), and non-viral vectors, such as liposomes. It is not intended that the present invention be limited to any specific gene therapy vector, as any vehicle suitable for a given setting finds use. The gene therapy vector may be designed to deliver genes to a specific species or host, or may find more general applicability.

“Gene therapy” refers to the delivery of a gene, polydeoxyribonucleotide, or polynucleotide sequence(s) with a gene therapy vector to cells or tissues for the modification of those cells or tissues for the treatment of prevention of a disease. Gene therapy may include replacing a mutated gene that causes disease with a healthy copy of the gene or a functioning variant of the gene, or inactivating, or “knocking out,” a mutated gene that is functioning improperly. In some embodiments, gene therapy is used in the treatment of disease in patients.

“mRNA therapy” refers to the delivery of an mRNA polyribonucleotide sequence to cells or tissues for the modification of those cells or tissues for the treatment or prevention of a disease. In some embodiments, the mRNA polynucleotide sequences for delivery to cells or tissue, are formulated, for instance, but not limited to, in liposomes. In some embodiments, mRNA therapy is used in the treatment of disease in patients.

“Cell therapy” refers to the delivery of living cells that have been modified exogenously to patients to provide a missing gene for the treatment or prevention of a disease. The modified cells are then reintroduced into the body.

“Composition” and “formulation” encompass products comprising at least one engineered acid alpha-glucosidase of the present disclosure, intended for any suitable use (e.g., pharmaceutical compositions, dietary/nutritional supplements, feed, etc.).

“Pharmaceutical composition” refers to a composition suitable for pharmaceutical use in a mammalian subject (e.g., human) comprising a pharmaceutically effective amount of an engineered acid alpha-glucosidase polypeptide encompassed by the invention and an acceptable carrier or excipient. In some embodiments, the pharmaceutical composition comprises a recombinant polynucleotide encoding an engineered acid alpha-glucosidase, for example, in the form of a gene therapy vector.

“Pharmaceutically acceptable” means a material that can be administered to a subject without causing any undesirable biological effects or interacting in a deleterious manner with any of the components in which it is contained and that possesses the desired biological activity.

“Carrier” when used in reference to a pharmaceutical composition means any of the standard pharmaceutical carrier, buffers, and excipients, such as stabilizers, preservatives, and adjuvants.

“Excipient” refers to any pharmaceutically acceptable additive, carrier, diluent, adjuvant, or other ingredient, other than the active pharmaceutical ingredient (API; e.g., the engineered acid alpha-glucosidase polypeptide or a recombinant polynucleotide encoding the acid alpha-glucosidase).

Excipients are typically included for formulation and/or administration purposes.

“Administration” and “administering” a composition mean providing a composition of the present invention to a subject (e.g., to a person suffering from the effects of Pompe disease).

“Effective amount” means an amount sufficient to produce the desired result. One of general skill in the art may determine what the effective amount by using routine experimentation.

“Therapeutically effective amount” when used in reference to symptoms of disease/condition refers to the amount and/or concentration of a compound (e.g., engineered acid alpha-glucosidase polypeptide or a recombinant polynucleotide encoding the engineered acid alpha-glucosidase) that ameliorates, attenuates, or eliminates one or more symptom of a disease/condition or prevents or delays the onset of symptom(s). A “therapeutically effective amount” when used in reference to a disease/condition refers to the amount and/or concentration of a composition (e.g., engineered GAA polypeptide or recombinant polynucleotide encoding the engineered acid alpha-glucosidase) that ameliorates, attenuates, or eliminates the disease/condition. In some embodiments, the term is use in reference to the amount of a composition that elicits the biological (e.g., medical) response by a tissue, system, or animal subject that is sought by the researcher, physician, veterinarian, or other clinician.

“Treating” or “treatment” of a disease, disorder, or syndrome, as used herein, includes (i) preventing the disease, disorder, or syndrome from occurring in a subject, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As such, the terms “treating,” “treat” and “treatment” encompass preventative (e.g., prophylactic), as well as palliative treatment. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

“Subject” encompasses mammals such as humans, non-human primates, livestock, companion animals, and laboratory animals (e.g., rodents and lagomorphs). It is intended that the term encompass females as well as males.

“Patient” means any subject that is being assessed for, treated for, or is experiencing disease.

“Infant” refers to a child in the period of the first month after birth to approximately one (1) year of age. As used herein, the term “newborn” refers to child in the period from birth to the 28th day of life. The term “premature infant” refers to an infant born after the twentieth completed week of gestation, yet before full term, generally weighing ˜500 to ˜2499 grams at birth. A “very low birth weight infant” is an infant weighing less than 1500 g at birth.

“Child” refers to a person who has not attained the legal age for consent to treatment or research procedures. In some embodiments, the term refers to a person between the time of birth and adolescence.

“Adult” refers to a person who has attained legal age for the relevant jurisdiction (e.g., 18 years). In some embodiments, the term refers to any fully grown, mature organism. In some embodiments, the term “young adult” refers to a person less than 18 years of age, but who has reached sexual maturity.

### Engineered GAA Polypeptides

The present disclosure provides engineered acid alpha-glucosidase polypeptides characterized by improved properties compared to the wild-type acid alpha-glucosidase or a reference engineered acid alpha-glucosidase polypeptide. The engineered acid alpha-glucosidase polypeptides described herein have been engineered to have, among others, improved activity, plasma stability, cellular uptake, stability in lysosomes, and/or acid pH stability. The engineered acid alpha-glucosidase polypeptides find use in therapeutic applications, such as for the treatment of conditions associated with deficiency in acid alpha-glucosidase.

In one aspect, the present disclosure provides an engineered acid alpha-glucosidase polypeptide, or biologically active fragment thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-944 of an even numbered SEQ ID NO. of SEQ ID NOs: 2, 12, and 14-754, or to an even numbered SEQ ID NO. of SEQ ID NOs: 2, 12, and 14-754, wherein the amino acid sequence comprises one or more substitutions relative to the reference sequence corresponding to residues 20-944 of SEQ ID NO: 12 or 2, or relative to the reference sequence corresponding to SEQ ID NO: 12 or 2.

In some embodiments, the engineered acid alpha-glucosidase, or biologically active fragment thereof, comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-944 of SEQ ID NO: 12 or 2, or to a reference sequence corresponding to SEQ ID NO: 12 or 2, wherein the amino acid sequence comprises one or more substitutions relative to the reference sequence corresponding to residues 20-944 of SEQ ID NO: 12 or 2, or relative to the reference sequence corresponding to SEQ ID NO: 12 or 2.

In some embodiments, the engineered acid alpha-glucosidase, or biologically active fragment thereof, comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-944 of SEQ ID NO: 12, or to the reference sequence corresponding to SEQ ID NO: 12, wherein the amino acid sequence comprises one or more substitutions relative to the reference sequence corresponding to residues 20-944 of SEQ ID NO: 12, or relative to the reference sequence corresponding to SEQ ID NO: 12.

In some embodiments, the engineered acid alpha-glucosidase, or biologically active fragment thereof, comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-944 of an even numbered SEQ ID NO. of SEQ ID NOs: 14-754, or to an even numbered SEQ ID NO. of SEQ ID NOs: 14-754, wherein the amino acid sequence comprises one or more substitutions relative to the reference sequence corresponding to residues 20-944 of SEQ ID NO: 12 or 2, or relative to the reference sequence corresponding to SEQ ID NO: 12 or 2.

In some embodiments, the amino acid sequence of the engineered acid alpha-glucosidase comprises at least a substitution at amino acid position 24, 28, 29, 39, 50, 62, 78, 87, 135, 150, 266, 267, 305, 437, 486, 522, 569, 670, 692, 711, 736, 750, 812, 830, 842, 871, 883, 894, 913, or 932, or combinations thereof, wherein the amino acid positions are relative to the reference sequence corresponding to residues 20-944 of SEQ ID NO: 12 or 2, or relative to the reference sequence corresponding to SEQ ID NO: 12 or 2.

In some embodiments, the amino acid sequence of the engineered acid alpha-glucosidase comprises at least a substitution or amino acid residue 24A/C/D/E/F/G/H/I/K/L/M/N/P/R/S/T/V/Y, 28A/C/D/E/F/G/H/K/L/P/Q/R/T/V/W, 29A/C/D/E/F/G/H/I/K/L/M/N/P/Q/R/S/V/W/Y, 39A/E/F/G/I/L/N/T, 50A/C/D/E/F/G/H/I/K/L/M/N/Q/R/S/T/W/Y, 62A/D/E/F/G/H/I/K/M/N/P/Q/S/T/V/Y, 78A/C/D/F/G/H/I/K/L/M/N/P/Q/R/S/T/V/W/Y, 87A/D/G/H/I/K/L/MN/Q/R/S/T/V/W, 135A/C/D/E/F/G/H/I/K/L/N/P/R/Y, 150T, 266A/D/E/H/K/Q/T, 267H/L/R/T/V, 305V, 437A/H/S, 486A/C/D/F/G/H/I/K/L/M/N/Q/R/S/T/V/W/Y, 522A/C/D/E/F/G/H/I/K/L/M/N/P/Q/R/S/T/W/Y, 569A/C/D/E/G/H/I/K/L/M/N/P/Q/R/S/V/W/Y, 670A/D/E/F/G/H/I/K/L/M/N/Q/R/S/V/Y, 692A/C/D/E/F/H/I/K/L/M/N/Q/R/S/T/V/W/Y, 711A/C/D/E/F/G/I/K/L/M/N/Q/R/S/T/V/W/Y, 736F/L, 750A/E/K/L/Q/R, 812A/D/G/S, 830D/E/F/G/H/L/M/N/Q/S/T/V/W/Y, 842A/C/D/E/F/G/H/K/L/M/N/Q/R/T/W, 871A/C/D/F/G/H/I/K/L/M/N/Q/R/S/T/V/W/Y, 883A/F/Q, 894A/C/D/E/H/I/K/L/M/N/Q/R/S/T/V/W/Y, 913E/F/H/I/K/M/N/Q/S/W, or 932C/D/E/G/H/K/L/M/N/P/Q/R/S/T/W/Y, or combinations thereof, wherein the amino acid positions are relative to the reference sequence corresponding to residues 20-944 of SEQ ID NO: 12 or 2, or relative to the reference sequence corresponding to SEQ ID NO: 12 or 2.

In some embodiments, the amino acid sequence of the engineered acid alpha-glucosidase comprises at least a substitution W24E/A/C/D/F/G/H/I/K/L/M/N/P/R/ST/V/Y, S28A/C/D/E/F/G/H/K/L/P/Q/R/T/V/W, T29A/C/D/E/F/G/H/I/K/L/M/N/P/Q/R/S/V/W/Y, Q39A/E/F/G/I/L/N/T, V50A/C/D/E/F/G/H/I/K/L/M/N/Q/R/S/T/W/Y, L62A/D/E/F/G/H/I/K/M/N/P/Q/S/T/V/Y, E78A/C/D/F/G/H/I/K/L/M/N/P/Q/R/S/T/V/W/Y, E87A/D/G/H/IK/L/M/N/Q/R/S/T/V/W, Q135A/C/D/E/F/G/H/I/K/L/N/P/R/Y, S150T, N266A/D/E/H/K/Q/T, K267H/L/R/T/V, G437A/H/S, E486A/C/D/F/G/H/I/K/L/M/N/Q/R/S/T/V/W/Y, V522A/C/D/E/F/G/H/I/K/L/M/N/P/Q/R/S/T/W/Y, T569A/C/D/E/G/H/I/K/L/M/N/P/Q/R/S/V/W/Y, T670A/D/E/F/G/H/I/K/L/M/N/Q/R/S/V/Y, G692A/C/D/E/F/H/I/K/L/M/N/Q/R/S/T/V/W/Y, H711A/C/D/E/F/G/I/K/L/M/N/Q/R/S/T/V/W/Y, M736F/L, P750A/E/K/L/Q/R, E812A/D/G/S, K830D/E/F/G/H/L/M/N/Q/S/T/V/W/Y, S842A/C/D/E/F/G/H/K/L/M/N/Q/R/T/W, E871A/C/D/F/G/H/I/K/L/M/N/Q/R/S/T/V/W/Y, H883A/F/Q, G894A/C/D/E/H/I/K/L/M/N/Q/R/S/T/V/W/Y, R913E/F/H/I/K/M/N/Q/S/W, A932C/D/E/G/H/K/L/M/N/P/Q/R/S/T/W/Y, or combinations thereof, wherein the amino acid positions are relative to the reference sequence corresponding to residues 20-944 of SEQ ID NO: 12 or 2, or relative to the reference sequence corresponding to SEQ ID NO: 12 or 2.

In some embodiments, the amino acid sequence of the engineered acid alpha-glucosidase comprises at least a substitution or amino acid residue 24A/C/D/F/G/H/I/K/M/N/P/S/T/V/Y, 28A/C/D/E/F/G/H/K/Q/T/V/W, 29A/C/D/E/F/G/H/I/K/M/N/P/R/W/Y, 39A/E/F/G/I/L/N/T, 50A/C/D/E/F/H/II/K/M/N/R/S/T/W/Y, 62D/H/I/K/M/N/P/Q/Y, 78A/C/D/F/G/H/I/K/L/M/N/Q/R/S/T/V/W/Y, 87A/G/H/I/K/L/MN/Q/R/S/T/V/W, 135C/D/E/F/G/H/I/K/L/N/R/Y, 266A/D/E/H/K/Q, 267H/L/T/V, 305V, 437A/H, 486C/D/F/G/H/I/K/L/M/N/Q/R/S/V/W/Y, 522A/C/D/F/G/H/I/K/L/M/N/P/Q/R/S/T/W/Y, 569A/C/D/E/G/K/M/N/P/R/W, 670A/D/G/H/K/M/Y, 692A/D/E/H/K/L/M/N/T/W, 711D/E/I/K/M/N/Q/S/T/V/Y, 736F/L, 750E/K/L/Q/R, 812A/D/G/S, 830D/E/F/G/H/L/M/N/S/T/W/Y, 842A/C/D/F/H/K/L/M/N/Q/R/T/W, 871A/C/D/F/H/I/M/N/Q/T/V/W/Y, 883A/F/Q, 894A/D/E/H/I/K/L/M/N/S/T/V/W/Y, 913F/IK/M/N/S, or 932C/D/E/G/H/K/L/M/N/P/Q/R/W/Y, or combinations thereof, wherein the amino acid positions are relative to the reference sequence corresponding to residues 20-944 of SEQ ID NO: 12, or relative to the reference sequence corresponding to SEQ ID NO: 12.

In some embodiments, the amino acid sequence of the engineered acid alpha-glucosidase comprises at least a substitution W24A/C/D/F/G/H/I/K/M/N/P/S/T/V/Y, S28A/C/D/E/F/G/H/K/Q/T/V/W, T29A/C/D/E/F/G/H/I/K/M/N/P/R/W/Y, Q39A/E/F/G/I/L/N/T, V50A/C/D/E/F/H/I/K/M/N/R/S/T/W/Y, L62D/H/I/K/M/N/P/Q/Y, E78A/C/D/F/G/H/I/K/L/M/N/Q/R/S/T/V/W/Y, E87A/G/H/I/K/L/MN/Q/R/S/T/V/W, Q135C/D/E/F/G/H/I/K/L/N/R/Y, N266A/D/E/H/K/Q, K267H/L/T/V, L305V, G437A/H, E486C/D/F/G/H/I/K/L/M/N/Q/R/S/V/W/Y, V522A/C/D/F/G/H/I/K/L/M/N/P/Q/R/S/T/W/Y, T569A/C/D/E/G/K/M/N/P/R/W, T670A/D/G/H/K/M/Y, G692A/D/E/H/K/L/M/N/T/W, H711D/E/I/K/M/N/Q/S/T/V/Y, M736F/L, P750E/K/L/Q/R, E812A/D/G/S, K830D/E/F/G/H/L/M/N/S/T/W/Y, S842A/C/D/F/H/K/L/M/N/Q/R/T/W, E871A/C/D/F/H/I/M/N/Q/T/V/W/Y, H883A/F/Q, G894A/D/E/H/IK/L/M/N/S/T/V/W/Y, R913F/I/K/M/N/S, or A932C/D/E/G/H/K/L/M/N/P/Q/R/W/Y, or combinations thereof, wherein the amino acid positions are relative to the reference sequence corresponding to residues 20-944 of SEQ ID NO: 12, or relative to the reference sequence corresponding to SEQ ID NO: 12.

In some embodiments, the amino acid sequence of the engineered acid alpha-glucosidase comprises at least a substitution at amino acid position 305. In some embodiments, the amino acid sequence of the engineered acid alpha-glucosidase comprises at least a substitution or amino acid residue 305V. In some embodiments, the amino acid sequence of the engineered acid alpha-glucosidase comprises at least a substitution L305V.

In some embodiments, the amino acid sequence of the engineered acid alpha-glucosidase comprises at least a substitution at amino acid position 24. In some embodiments, the amino acid sequence of the engineered acid alpha-glucosidase comprises at least a substitution or amino acid residue 24A/C/D/F/G/H/I/K/M/N/P/S/T/V/Y. In some embodiments, the amino acid sequence of the engineered acid alpha-glucosidase comprises at least a substitution or amino acid residue 24A/C/D/E/F/G/H/I/K/L/M/N/P/R/ST/V/Y. In some embodiments, the amino acid sequence of the engineered acid alpha-glucosidase comprises at least a substitution W24A/C/D/F/G/H/I/K/M/N/P/S/T/V/Y. In some embodiments, the amino acid sequence of the engineered acid alpha-glucosidase comprises at least a substitution W24E/A/C/D/F/G/H/I/K/L/M/N/P/R/ST/V/Y.

In some embodiments, the amino acid sequence of the engineered acid alpha-glucosidase comprises at least a substitution at amino acid position 28. In some embodiments, the amino acid sequence of the engineered acid alpha-glucosidase comprises at least a substitution or amino acid residue 28A/C/D/E/F/G/H/K/Q/T/V/W. In some embodiments, the amino acid sequence of the engineered acid alpha-glucosidase comprises at least a substitution or amino acid residue 28A/C/D/E/F/G/H/K/L/P/Q/R/T/V/W. In some embodiments, the amino acid sequence of the engineered acid alpha-glucosidase comprises at least a substitution S28A/C/D/E/F/G/H/K/Q/T/V/W. In some embodiments, the amino acid sequence of the engineered acid alpha-glucosidase comprises at least a substitution S28A/C/D/E/F/G/H/K/L/P/Q/R/T/V/W.

In some embodiments, the amino acid sequence of the engineered acid alpha-glucosidase comprises at least a substitution at amino acid position 29. In some embodiments, the amino acid sequence of the engineered acid alpha-glucosidase comprises at least a substitution or amino acid residue 29A/C/D/E/F/G/H/I/K/M/N/P/R/W/Y. In some embodiments, the amino acid sequence of the engineered acid alpha-glucosidase comprises at least a substitution or amino acid residue 29A/C/D/E/F/G/H/I/K/L/M/N/P/Q/R/S/V/W/Y. In some embodiments, the amino acid sequence of the engineered acid alpha-glucosidase comprises at least a substitution T29A/C/D/E/F/G/H/I/K/M/N/P/R/W/Y. In some embodiments, the amino acid sequence of the engineered acid alpha-glucosidase comprises at least a substitution T29A/C/D/E/F/G/H/I/K/L/M/N/P/Q/R/S/V/W/Y.

In some embodiments, the amino acid sequence of the engineered acid alpha-glucosidase comprises at least a substitution at amino acid position 39. In some embodiments, the amino acid sequence of the engineered acid alpha-glucosidase comprises at least a substitution or amino acid residue 39A/E/F/G/I/L/N/T. In some embodiments, the amino acid sequence of the engineered acid alpha-glucosidase comprises at least a substitution Q39A/E/F/G/I/L/N/T.

In some embodiments, the amino acid sequence of the engineered acid alpha-glucosidase comprises at least a substitution at amino acid position 50. In some embodiments, the amino acid sequence of the engineered acid alpha-glucosidase comprises at least a substitution or amino acid residue 50A/C/D/E/F/H/II/K/M/N/R/S/T/W/Y. In some embodiments, the amino acid sequence of the engineered acid alpha-glucosidase comprises at least a substitution or amino acid residue 50A/C/D/E/F/G/H/I/K/L/M/N/Q/R/S/T/W/Y. In some embodiments, the amino acid sequence of the engineered acid alpha-glucosidase comprises at least a substitution V50A/C/D/E/F/H/I/K/M/N/R/S/T/W/Y. In some embodiments, the amino acid sequence of the engineered acid alpha-glucosidase comprises at least a substitution V50A/C/D/E/F/G/H/I/K/L/M/N/Q/R/S/T/W/Y.

In some embodiments, the amino acid sequence of the engineered acid alpha-glucosidase comprises at least a substitution at amino acid position 62. In some embodiments, the amino acid sequence of the engineered acid alpha-glucosidase comprises at least a substitution or amino acid residue 62D/H/I/K/M/N/P/Q/Y. In some embodiments, the amino acid sequence of the engineered acid alpha-glucosidase comprises at least a substitution or amino acid residue 62A/D/E/F/G/H/I/K/M/N/P/Q/S/T/V/Y. In some embodiments, the amino acid sequence of the engineered acid alpha-glucosidase comprises at least a substitution L62D/H/I/K/M/N/P/Q/Y. In some embodiments, the amino acid sequence of the engineered acid alpha-glucosidase comprises at least a substitution L62A/D/E/F/G/H/I/K/M/N/P/Q/S/T/V/Y.

In some embodiments, the amino acid sequence of the engineered acid alpha-glucosidase comprises at least a substitution at amino acid position 78. In some embodiments, the amino acid sequence of the engineered acid alpha-glucosidase comprises at least a substitution or amino acid residue 78A/C/D/F/G/H/I/K/L/M/N/Q/R/S/T/V/W/Y. In some embodiments, the amino acid sequence of the engineered acid alpha-glucosidase comprises at least a substitution or amino acid residue 78A/C/D/F/G/H/I/K/L/M/N/P/Q/R/S/T/V/W/Y. In some embodiments, the amino acid sequence of the engineered acid alpha-glucosidase comprises at least a substitution E78A/C/D/F/G/H/I/K/L/M/N/Q/R/S/T/V/W/Y. In some embodiments, the amino acid sequence of the engineered acid alpha-glucosidase comprises at least a substitution E78A/C/D/F/G/H/I/K/L/M/N/P/Q/R/S/T/V/W/Y.

In some embodiments, the amino acid sequence of the engineered acid alpha-glucosidase comprises at least a substitution at amino acid position 87. In some embodiments, the amino acid sequence of the engineered acid alpha-glucosidase comprises at least a substitution or amino acid residue 87A/G/H/I/K/L/MN/Q/R/S/T/V/W. In some embodiments, the amino acid sequence of the engineered acid alpha-glucosidase comprises at least a substitution or amino acid residue 87A/D/G/H/I/K/L/M/N/Q/R/S/T/V/W. In some embodiments, the amino acid sequence of the engineered acid alpha-glucosidase comprises at least a substitution E87A/G/H/I/K/L/MN/Q/R/S/T/V/W.

In some embodiments, the amino acid sequence of the engineered acid alpha-glucosidase comprises at least a substitution E87A/D/G/H/I/K/L/M/N/Q/R/S/T/V/W.

In some embodiments, the amino acid sequence of the engineered acid alpha-glucosidase comprises at least a substitution at amino acid position 135. In some embodiments, the amino acid sequence of the engineered acid alpha-glucosidase comprises at least a substitution or amino acid residue 135C/D/E/F/G/H/I/K/L/N/R/Y. In some embodiments, the amino acid sequence of the engineered acid alpha-glucosidase comprises at least a substitution or amino acid residue 135A/C/D/E/F/G/H/I/K/L/N/P/R/Y. In some embodiments, the amino acid sequence of the engineered acid alpha-glucosidase comprises at least a substitution Q135C/D/E/F/G/H/I/K/L/N/R/Y. In some embodiments, the amino acid sequence of the engineered acid alpha-glucosidase comprises at least a substitution Q135A/C/D/E/F/G/H/I/K/L/N/P/R/Y.

In some embodiments, the amino acid sequence of the engineered acid alpha-glucosidase comprises at least a substitution at amino acid position 150. In some embodiments, the amino acid sequence of the engineered acid alpha-glucosidase comprises at least a substitution or amino acid residue 150T. In some embodiments, the amino acid sequence of the engineered acid alpha-glucosidase comprises at least a substitution S150T.

In some embodiments, the amino acid sequence of the engineered acid alpha-glucosidase comprises at least a substitution at amino acid position 266. In some embodiments, the amino acid sequence of the engineered acid alpha-glucosidase comprises at least a substitution or amino acid residue 266A/D/E/H/K/Q. In some embodiments, the amino acid sequence of the engineered acid alpha-glucosidase comprises at least a substitution or amino acid residue 266A/D/E/H/K/Q/T. In some embodiments, the amino acid sequence of the engineered acid alpha-glucosidase comprises at least a substitution N266A/D/E/H/K/Q. In some embodiments, the amino acid sequence of the engineered acid alpha-glucosidase comprises at least a substitution N266A/D/E/H/K/Q/T.

In some embodiments, the amino acid sequence of the engineered acid alpha-glucosidase comprises at least a substitution at amino acid position 267. In some embodiments, the amino acid sequence of the engineered acid alpha-glucosidase comprises at least a substitution or amino acid residue 267H/L/T/V. In some embodiments, the amino acid sequence of the engineered acid alpha-glucosidase comprises at least a substitution or amino acid residue 267H/L/R/T/V. In some embodiments, the amino acid sequence of the engineered acid alpha-glucosidase comprises at least a substitution K267H/L/T/V. In some embodiments, the amino acid sequence of the engineered acid alpha-glucosidase comprises at least a substitution K267H/L/R/T/V.

In some embodiments, the amino acid sequence of the engineered acid alpha-glucosidase comprises at least a substitution at amino acid position 437. In some embodiments, the amino acid sequence of the engineered acid alpha-glucosidase comprises at least a substitution or amino acid residue 437H. In some embodiments, the amino acid sequence of the engineered acid alpha-glucosidase comprises at least a substitution or amino acid residue 437A/H/S. In some embodiments, the amino acid sequence of the engineered acid alpha-glucosidase comprises at least a substitution G437H. In some embodiments, the amino acid sequence of the engineered acid alpha-glucosidase comprises at least a substitution G437A/H/S.

In some embodiments, the amino acid sequence of the engineered acid alpha-glucosidase comprises at least a substitution at amino acid position 486. In some embodiments, the amino acid sequence of the engineered acid alpha-glucosidase comprises at least a substitution or amino acid residue 486C/D/F/G/H/I/K/L/M/N/Q/R/S/V/W/Y. In some embodiments, the amino acid sequence of the engineered acid alpha-glucosidase comprises at least a substitution or amino acid residue 486A/C/D/F/G/H/I/K/L/M/N/Q/R/S/T/V/W/Y. In some embodiments, the amino acid sequence of the engineered acid alpha-glucosidase comprises at least a substitution E486C/D/F/G/H/I/K/L/M/N/Q/R/S/V/W/Y. In some embodiments, the amino acid sequence of the engineered acid alpha-glucosidase comprises at least a substitution E486A/C/D/F/G/H/I/K/L/M/N/Q/R/S/T/V/W/Y.

In some embodiments, the amino acid sequence of the engineered acid alpha-glucosidase comprises at least a substitution at amino acid position 522. In some embodiments, the amino acid sequence of the engineered acid alpha-glucosidase comprises at least a substitution or amino acid residue 522A/C/D/F/G/H/I/K/L/M/N/P/Q/R/S/T/W/Y. In some embodiments, the amino acid sequence of the engineered acid alpha-glucosidase comprises at least a substitution or amino acid residue 522A/C/D/E/F/G/H/I/K/L/M/N/P/Q/R/S/T/W/Y. In some embodiments, the amino acid sequence of the engineered acid alpha-glucosidase comprises at least a substitution V522A/C/D/F/G/H/I/K/L/M/N/P/Q/R/S/T/W/Y. In some embodiments, the amino acid sequence of the engineered acid alpha-glucosidase comprises at least a substitution V522A/C/D/E/F/G/H/I/K/L/M/N/P/Q/R/S/T/W/Y.

In some embodiments, the amino acid sequence of the engineered acid alpha-glucosidase comprises at least a substitution at amino acid position 569. In some embodiments, the amino acid sequence of the engineered acid alpha-glucosidase comprises at least a substitution or amino acid residue 569A/C/D/E/G/K/M/N/P/R/W. In some embodiments, the amino acid sequence of the engineered acid alpha-glucosidase comprises at least a substitution or amino acid residue 569A/C/D/E/G/H/I/K/L/M/N/P/Q/R/S/V/W/Y. In some embodiments, the amino acid sequence of the engineered acid alpha-glucosidase comprises at least a substitution T569A/C/D/E/G/K/M/N/P/R/W. In some embodiments, the amino acid sequence of the engineered acid alpha-glucosidase comprises at least a substitution T569A/C/D/E/G/H/I/K/L/M/N/P/Q/R/S/V/W/Y.

In some embodiments, the amino acid sequence of the engineered acid alpha-glucosidase comprises at least a substitution at amino acid position 670. In some embodiments, the amino acid sequence of the engineered acid alpha-glucosidase comprises at least a substitution or amino acid residue 670A/D/G/H/K/M/Y. In some embodiments, the amino acid sequence of the engineered acid alpha-glucosidase comprises at least a substitution or amino acid residue 670A/D/E/F/G/H/I/K/L/M/N/Q/R/S/V/Y. In some embodiments, the amino acid sequence of the engineered acid alpha-glucosidase comprises at least a substitution T670A/D/G/H/K/M/Y. In some embodiments, the amino acid sequence of the engineered acid alpha-glucosidase comprises at least a substitution T670A/D/E/F/G/H/I/K/L/M/N/Q/R/S/V/Y.

In some embodiments, the amino acid sequence of the engineered acid alpha-glucosidase comprises at least a substitution at amino acid position 692. In some embodiments, the amino acid sequence of the engineered acid alpha-glucosidase comprises at least a substitution or amino acid residue 692A/D/E/H/K/L/M/N/T/W. In some embodiments, the amino acid sequence of the engineered acid alpha-glucosidase comprises at least a substitution or amino acid residue 692A/C/D/F/H/I/K/L/M/N/Q/R/S/T/V/W/Y/E. In some embodiments, the amino acid sequence of the engineered acid alpha-glucosidase comprises at least a substitution G692A/D/E/H/K/L/M/N/T/W. In some embodiments, the amino acid sequence of the engineered acid alpha-glucosidase comprises at least a substitution G692A/C/D/E/F/H/I/K/L/M/N/Q/R/S/T/V/W/Y.

In some embodiments, the amino acid sequence of the engineered acid alpha-glucosidase comprises at least a substitution at amino acid position 711. In some embodiments, the amino acid sequence of the engineered acid alpha-glucosidase comprises at least a substitution or amino acid residue 711D/E/I/K/M/N/Q/S/T/V/Y. In some embodiments, the amino acid sequence of the engineered acid alpha-glucosidase comprises at least a substitution or amino acid residue 711A/C/D/E/F/G/I/K/L/M/N/Q/R/S/T/V/W/Y. In some embodiments, the amino acid sequence of the engineered acid alpha-glucosidase comprises at least a substitution H711D/E/I/K/M/N/Q/S/T/V/Y. In some embodiments, the amino acid sequence of the engineered acid alpha-glucosidase comprises at least a substitution H711A/C/D/E/F/G/I/K/L/M/N/Q/R/S/T/V/W/Y.

In some embodiments, the amino acid sequence of the engineered acid alpha-glucosidase comprises at least a substitution at amino acid position 736. In some embodiments, the amino acid sequence of the engineered acid alpha-glucosidase comprises at least a substitution or amino acid residue 736F/L. In some embodiments, the amino acid sequence of the engineered acid alpha-glucosidase comprises at least a substitution M736F/L.

In some embodiments, the amino acid sequence of the engineered acid alpha-glucosidase comprises at least a substitution at amino acid position 750. In some embodiments, the amino acid sequence of the engineered acid alpha-glucosidase comprises at least a substitution or amino acid residue 750E/K/L/Q/R. In some embodiments, the amino acid sequence of the engineered acid alpha-glucosidase comprises at least a substitution or amino acid residue 750A/E/K/L/Q/R. In some embodiments, the amino acid sequence of the engineered acid alpha-glucosidase comprises at least a substitution P750E/K/L/Q/R. In some embodiments, the amino acid sequence of the engineered acid alpha-glucosidase comprises at least a substitution P750A/E/K/L/Q/R.

In some embodiments, the amino acid sequence of the engineered acid alpha-glucosidase comprises at least a substitution at amino acid position 812. In some embodiments, the amino acid sequence of the engineered acid alpha-glucosidase comprises at least a substitution or amino acid residue 812A/D/G/S. In some embodiments, the amino acid sequence of the engineered acid alpha-glucosidase comprises at least a substitution E812A/D/G/S.

In some embodiments, the amino acid sequence of the engineered acid alpha-glucosidase comprises at least a substitution at amino acid position 830. In some embodiments, the amino acid sequence of the engineered acid alpha-glucosidase comprises at least a substitution or amino acid residue 830D/E/F/G/H/L/M/N/S/T/W/Y. In some embodiments, the amino acid sequence of the engineered acid alpha-glucosidase comprises at least a substitution or amino acid residue 830D/E/F/G/H/L/M/N/Q/S/T/V/W/Y. In some embodiments, the amino acid sequence of the engineered acid alpha-glucosidase comprises at least a substitution K830D/E/F/G/H/L/M/N/S/T/W/Y. In some embodiments, the amino acid sequence of the engineered acid alpha-glucosidase comprises at least a substitution K830D/E/F/G/H/L/M/N/Q/S/T/V/W/Y.

In some embodiments, the amino acid sequence of the engineered acid alpha-glucosidase comprises at least a substitution at amino acid position 842. In some embodiments, the amino acid sequence of the engineered acid alpha-glucosidase comprises at least a substitution or amino acid residue 842A/C/D/F/H/K/L/M/N/Q/R/T/W. In some embodiments, the amino acid sequence of the engineered acid alpha-glucosidase comprises at least a substitution or amino acid residue 842A/C/D/E/F/G/H/K/L/M/N/Q/R/T/W. In some embodiments, the amino acid sequence of the engineered acid alpha-glucosidase comprises at least a substitution S842A/C/D/F/H/K/L/M/N/Q/R/T/W.

In some embodiments, the amino acid sequence of the engineered acid alpha-glucosidase comprises at least a substitution S842A/C/D/E/F/G/H/K/L/M/N/Q/R/T/W.

In some embodiments, the amino acid sequence of the engineered acid alpha-glucosidase comprises at least a substitution at amino acid position 871. In some embodiments, the amino acid sequence of the engineered acid alpha-glucosidase comprises at least a substitution or amino acid residue 871A/C/D/F/H/I/M/N/Q/T/V/W/Y. In some embodiments, the amino acid sequence of the engineered acid alpha-glucosidase comprises at least a substitution or amino acid residue 871A/C/D/F/G/H/I/K/L/M/N/Q/R/S/T/V/W/Y. In some embodiments, the amino acid sequence of the engineered acid alpha-glucosidase comprises at least a substitution E871A/C/D/F/H/I/M/N/Q/T/V/W/Y.

In some embodiments, the amino acid sequence of the engineered acid alpha-glucosidase comprises at least a substitution E871A/C/D/F/G/H/I/K/L/M/N/Q/R/S/T/V/W/Y.

In some embodiments, the amino acid sequence of the engineered acid alpha-glucosidase comprises at least a substitution at amino acid position 883. In some embodiments, the amino acid sequence of the engineered acid alpha-glucosidase comprises at least a substitution or amino acid residue 883A/F/Q. In some embodiments, the amino acid sequence of the engineered acid alpha-glucosidase comprises at least a substitution H883A/F/Q.

In some embodiments, the amino acid sequence of the engineered acid alpha-glucosidase comprises at least a substitution at amino acid position 894. In some embodiments, the amino acid sequence of the engineered acid alpha-glucosidase comprises at least a substitution or amino acid residue 894A/D/E/H/I/K/L/M/N/S/T/V/W/Y. In some embodiments, the amino acid sequence of the engineered acid alpha-glucosidase comprises at least a substitution or amino acid residue 894A/C/D/E/H/I/K/L/M/N/Q/R/S/T/V/W/Y. In some embodiments, the amino acid sequence of the engineered acid alpha-glucosidase comprises at least a substitution G894A/D/E/H/I/K/L/M/N/S/T/V/W/Y. In some embodiments, the amino acid sequence of the engineered acid alpha-glucosidase comprises at least a substitution G894A/C/D/E/H/I/K/L/M/N/Q/R/S/T/V/W/Y.

In some embodiments, the amino acid sequence of the engineered acid alpha-glucosidase comprises at least a substitution at amino acid position 913. In some embodiments, the amino acid sequence of the engineered acid alpha-glucosidase comprises at least a substitution or amino acid residue 913F/I/K/M/N/S. In some embodiments, the amino acid sequence of the engineered acid alpha-glucosidase comprises at least a substitution or amino acid residue 913E/F/H/I/K/M/N/Q/S/W. In some embodiments, the amino acid sequence of the engineered acid alpha-glucosidase comprises at least a substitution R913F/I/K/M/N/S. In some embodiments, the amino acid sequence of the engineered acid alpha-glucosidase comprises at least a substitution R913E/F/H/I/K/M/N/Q/S/W.

In some embodiments, the amino acid sequence of the engineered acid alpha-glucosidase comprises at least a substitution at amino acid position 932. In some embodiments, the amino acid sequence of the engineered acid alpha-glucosidase comprises at least a substitution or amino acid residue 932C/D/E/G/H/K/L/M/N/P/Q/R/W/Y. In some embodiments, the amino acid sequence of the engineered acid alpha-glucosidase comprises at least a substitution or amino acid residue 932C/D/E/G/H/K/L/M/N/P/Q/R/S/T/W/Y. In some embodiments, the amino acid sequence of the engineered acid alpha-glucosidase comprises at least a substitution A932C/D/E/G/H/K/L/M/N/P/Q/R/W/Y. In some embodiments, the amino acid sequence of the engineered acid alpha-glucosidase comprises at least a substitution A932C/D/E/G/H/K/L/M/N/P/Q/R/S/T/W/Y.

In some embodiments, the engineered acid alpha-glucosidase comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to an engineered acid alpha-glucosidase with a substitution or substitution set as set forth in Table 3-1, wherein the amino acid positions are relative to the reference sequence corresponding to residues 20-944 of SEQ ID NO: 12 or 2, or relative to the reference sequence corresponding to SEQ ID NO: 12 or 2.

In some embodiments, the amino acid sequence of the engineered acid alpha-glucosidase comprises at least a substitution at an amino acid position as set forth in Table 3-1, wherein the amino acid positions are relative to the reference sequence corresponding to residues 20-944 of SEQ ID NO: 12 or 2, or relative to the reference sequence corresponding to SEQ ID NO: 12 or 2.

In some embodiments, the amino acid sequence of the engineered acid alpha-glucosidase comprises at least one substitution as set forth in Table 3-1, wherein the amino acid positions are relative to the reference sequence corresponding to residues 20-944 of SEQ ID NO: 12 or 2, or relative to the reference sequence corresponding to SEQ ID NO: 12 or 2.

In some embodiments, the amino acid sequence of the engineered acid alpha-glucosidase comprises at least a substitution set as set forth in Table 3-1, wherein the amino acid positions are relative to the reference sequence corresponding to residues 20-944 of SEQ ID NO: 2, or relative to the reference sequence corresponding to SEQ ID NO: 2.

In some embodiments, the amino acid sequence of the engineered acid alpha-glucosidase comprises at least a substitution set at amino acid positions 24/28/29/39/50/62/78/87/135/150/266/267/305/437/486/522/569/670/692/711/736/750/812/830/842/871/883/894/913/932, 24/28/29/39/50/62/78/87/135/150/266/267/437/486/522/569/670/692/711/736/750/812/830/842/871/883/894/913/932, 24/28/29/39/50/62/78/87/135/150/266/267/437/486/522/569/670/692/711/736/750/812/830/871/883/894/913/932, 28/29/39/50/62/78/87/135/150/266/267/437/486/522/569/670/692/711/736/750/812/830/842/871/883/89 4/913/932, 24/28/29/39/50/78/87/135/150/266/267/437/486/522/569/670/692/711/736/750/812/830/842/871/883/89 4/913/932, 24/28/29/39/50/62/78/87/135/150/266/267/437/486/522/569/670/692/711/736/750/812/830/842/883/894/913/932, 24/28/29/39/50/62/78/87/135/150/266/267/437/486/522/670/692/711/736/750/812/830/842/871/883/894/913/932, 24/28/29/39/50/62/78/87/135/150/266/267/437/486/522/569/692/711/736/750/812/830/842/871/883/894/913/932, 24/28/29/39/50/62/78/87/135/150/266/267/437/486/522/569/670/692/736/750/812/830/842/871/883/894/913/932, 24/28/29/39/50/62/78/87/135/150/266/267/437/486/522/569/670/692/711/750/812/830/842/871/883/894/913/932, 24/28/29/39/50/62/78/87/135/150/266/267/437/486/569/670/692/711/736/750/812/830/842/871/883/894/913/932, 24/28/29/39/50/62/78/87/135/150/266/267/437/486/522/569/670/692/711/736/812/830/842/871/883/894/913/932, 24/28/39/50/62/78/87/135/150/266/267/437/486/522/569/670/692/711/736/750/812/830/842/871/883/89 4/913/932, 24/28/29/39/50/62/78/87/135/150/266/437/486/522/569/670/692/711/736/750/812/830/842/871/883/894/913/932, 24/29/39/50/62/78/87/135/150/266/267/437/486/522/569/670/692/711/736/750/812/830/842/871/883/89 4/913/932, 24/28/29/39/50/62/78/135/150/266/267/437/486/522/569/670/692/711/736/750/812/830/842/871/883/89 4/913/932, 24/28/29/39/50/62/78/87/135/150/266/267/486/522/569/670/692/711/736/750/812/830/842/871/883/894/913/932, 24/28/29/39/50/62/87/135/150/266/267/437/486/522/569/670/692/711/736/750/812/830/842/871/883/89 4/913/932, 24/28/29/39/50/62/78/87/135/150/266/267/437/486/522/569/670/692/711/736/750/812/830/8425/871/88 3/894/913/932, 24/28/29/39/50/62/78/87/135/266/267/437/486/522/569/670/692/711/736/750/812/830/842/871/883/894/913/932, 24/28/29/39/50/62/78/87/135/150/266/267/437/486/522/569/670/692/711/736/750/812/842/871/883/894/913/932, 24/28/29/39/50/62/78/87/135/150/266/267/437/486/522/569/670/692/711/736/750/830/842/871/883/894/913/932, 24/28/29/39/62/78/87/135/150/266/267/437/486/522/569/670/692/711/736/750/812/830/842/871/883/89 4/913/932, 24/28/29/39/50/62/78/87/135/150/266/267/437/486/522/569/670/711/736/750/812/830/842/871/883/894/913/932, 24/28/29/39/50/62/78/87/135/150/266/267/437/486/522/569/670/692/711/736/750/812/830/842/871/883/913/932, 24/28/29/39/50/62/78/87/135/150/267/437/486/522/569/670/692/711/736/750/812/830/842/871/883/894/913/932, 24/28/29/39/50/62/78/87/135/150/266/267/437/522/569/670/692/711/736/750/812/830/842/871/883/894/913/932, 24/28/29/39/50/62/78/87/135/150/266/267/437/486/522/569/670/692/711/736/750/812/830/842/871/883/894/913, or 24/28/29/39/50/62/78/87/135/150/266/267/437/486/522/569/670/692/711/736/750/8125/830/842/871/88 3/894/913/932, wherein the amino acid positions are relative to the reference sequence corresponding to residues 20-944 of SEQ ID NO: 2, or relative to the reference sequence corresponding to SEQ ID NO: 2. In some embodiments, the specific amino acid substitutions at the positions in the substitution set are selected from the substitutions described herein for each of the amino acid positions.

In some embodiments, the amino acid sequence of the engineered acid alpha-glucosidase comprises at least a substitution set 24W/28S/29T/39Q/50V/62L/78E/87E/135Q/150S/266N/267K/305V/437G/486E/522V/569T/670T/692G/711H/736M/750P/812E/830K/842S/871E/883H/894G/913R/932A, 24W/28S/29T/39Q/50V/62L/78E/87E/135P/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486Q/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842E/871E/883H/894G/913R/932A, 24W/28S/29T/39Q/50V/62L/78G/87E/135Q/150S/266N/267K/437G/486E/522V/569T/670T/692G/711 H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894S/913R/932A, 24W/28S/29A/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/871E/883H/894G/913R/932A, 24W/28S/29T/39Q/50V/62S/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932R, 24W/28A/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569A/670T/692G/711H/736M/750P/812E/830K/842S/871E/883H/894G/913R/932A, 24W/28S/29T/39Q/50V/62L/78E/87E/135Q/150S/266N/267K/437G/486E/522V/569R/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842W/871E/883H/894G/913R/932A, 24W/28S/29C/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267L/437G/486E/522V/569T/670T/692G/711H/736M/750P/812E/830K/842S/871E/883H/894G/913R/932A, 24W/28S/29T/39Q/50V/62L/78E/87Q/135Q/150S/266N/267K/437G/486E/522V/569T/670T/692G/711H/736M/750P/812E/830K/842S/871E/883H/894G/913R/932A, 24W/28S/29T/39Q/50V/62D/78E/87E/135Q/150S/266N/267K/437G/486E/522V/569T/670T/692G/711H/736M/750P/812E/830K/842S/871E/883H/894G/913R/932A, 24W/28S/29T/39Q/50V/62H/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135R/150S/266N/267K/437G/486E/522V/569T/670T/692G/711H/736M/750P/812E/830K/842S/871E/883H/894G/913R/932A, 28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871A/883H/894G/913R/932A, 24W/28S/29T/39Q/50S/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569G/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692R/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830N/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894R/913R/932A, 24W/28S/29T/39Q/50V/62L/78E/87E/135Q/150S/266N/267T/437G/486E/522V/569T/670T/692G/711H/736M/750P/812E/830K/842S/871E/883H/894G/913R/932A, 24E/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135G/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692V/711H/736M/750P/812E/830K/842S/871E/883H/894G/913R/932A, 24W/28S/29T/39Q/50V/62L/78E/87R/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913Q/932A, 24W/28S/29T/39Q/50V/62L/78E/87E/135Q/150S/266N/267K/437G/486E/522V/569T/670T/692G/711L/736M/750P/812E/830K/842S/871E/883H/894G/913R/932A, 24W/28S/29T/39Q/50V/62L/78E/87L/135Q/150S/266N/267K/437G/486E/522V/569T/670T/692G/711H/736M/750P/812E/830K/842S/871E/883H/894G/913R/932A, 24W/28S/29T/39Q/50V/62L/78E/87V/135Q/150S/266N/267K/437G/486E/522V/569T/670T/692G/711H/736M/750P/812E/830K/842S/871E/883H/894G/913R/932A, 24W/28S/29T/39Q/50V/62L/78Y/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569C/670T/692G/711H/736M/750P/812E/830K/842S/871E/883H/894G/913R/932A, 24W/28S/29T/39Q/50V/62L/78E/87W/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670A/692G/711H/736M/750P/812E/830K/842S/871E/883H/894G/913R/932A, 24W/28S/29T/39Q/50V/62L/78E/87E/135Q/150S/266N/267K/437G/486E/522V/569Q/670T/692G/711 H/736M/750P/812E/830K/842S/871E/883H/894G/913R/932A, 24W/28S/29T/39Q/50V/62K/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894C/913R/932A, 24W/28S/29T/39Q/50V/62L/78E/87E/135Q/150S/266N/267K/437G/486E/522V/569H/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711V/736M/750P/812E/830K/842S/871E/883H/894G/913R/932A, 24W/28S/29T/39Q/50V/62L/78E/87E/135Q/150S/266N/267K/437G/486E/522S/569T/670T/692G/711H/736M/750P/812E/830K/842S/871E/883H/894G/913R/932A, 24F/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569M/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711S/736M/750P/812E/830K/842S/871E/883H/894G/913R/932A, 24Y/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87G/135Q/150S/266N/267K/437G/486E/522V/569T/670T/692G/711H/736M/750P/812E/830K/842S/871E/883H/894G/913R/932A, 24W/28S/29T/39Q/50V/62L/78I/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/883H/894G/913R/932A, 24W/28S/29T/39Q/50V/62L/78R/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/670T/692G/711H/736M/750P/812E/830K/842S/871E/883H/894G/913R/932A, 24C/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/692G/711H1736M/750P/812E/830K/842S/871E/883H/894G/913R/932A, 24W/28S/29T/39Q/50V/62L/78W/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522F/569T/670T/692G/711H/736M/750P/812E/830K/842S/871E/883H/894G/913R/932A, 24W/28S/29T/39Q/50V/62L/78E/87E/135Q/150S/266N/267K/437G/486E/522P/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830V/842S/871E/883H/894G/913R/932A, 24W/28S/29T/39Q/50V/62L/78E/87E/135Q/150S/266N/267K/437G/486E/522V/569T/670T/692G/711M/736M/750P/812E/830K/842S/871E/883H/894G/913R/932A, 24W/28T/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569K/670T/692G/711H/736M/750P/812E/830K/842S/871E/883H/894G/913R/932A, 24S/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135L/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842R/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894Y/913R/932A, 24W/28S/29T/39Q/50V/62L/78E/87E/135Q/150S/266N/267K/437G/486E/522V/569T/670T/692G/711H/736M/750P/812E/830K/842T/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894T/913R/932A, 24W/28Q/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/736M/750P/812E/830K/842S/871E/883H/894G/913R/932A, 24W/28S/29T/39Q/50V/62L/78E/87E/135Q/150S/266H/267K/437G/486E/522V/569T/670T/692G/711H/736M/750P/812E/830K/842S/871E/883H/894G/913R/932A, 24W/28S/29T/39Q/50V/62P/78E/87E/135Q/150S/266N/267K/437G/486E/522V/569T/670T/692G/711H/736M/750P/812E/830K/842S/871E/883H/894G/913R/932A, 24W/28S/29T/39L/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486D/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711G/736M/750P/812E/830K/842S/871E/883H/894G/913R/932A, 24N/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711 W/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871R/883H/894G/913R/932A, 24W/28S/29T/39Q/50V/62L/78E/87E/135Q/150S/266N/267K/437G/486E/522V/569T/670T/692G/711H/750P/812E/830K/842S/871E/883H/894G/913R/932A, 24W/28S/29T/39Q/50V/62L/78E/87E/135Q/150S/266N/267K/437G/486E/522W/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894L/913R/932A, 24W/28S/29T/39Q/50V/62L/78E/87E/135Q/150S/266N/267K/437G/486E/522V/569T/670T/692G/711H/736M/750P/812E/830K/842S/871E/883A/894G/913R/932A, 24W/28S/29T/39Q/50V/62L/78E/87E/135Q/150S/266N/267K/437G/486E/522G/569T/670T/692G/711H/736M/750P/812E/830K/842S/871E/883H/894G/913R/932A, 24W/28S/29T/39Q/50V/62L/78E/87E/135Q/150S/266N/267K/437G/486E/522L/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670V/692G/711H/736M/750P/812E/830K/842S/871E/883H/894G/913R/932A, 24W/28S/29T/39Q/50V/62G/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692N/711H/736M/750P/812E/830K/842S/871E/883H/894G/913R/932A, 24W/28S/29T/39Q/50V/62L/78E/87E/135Q/150S/266N/267K/437G/486E/522V/569V/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670I/692G/711H/736M/750P/812E/830K/842S/871E/883H/894G/913R/932A, 24W/28S/29T/39Q/50V/62L/78E/87A/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894A/913R/932A, 24W/28S/29T/39Q/50V/62L/78E/87E/135Q/150S/266A/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/569T/670T/692G/711H/736M/750P/812E/830K/842S/871E/883H/894G/913R/932A, 24W/28R/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871G/883H/894G/913R/932A, 24W/28S/29T/39Q/50V/62L/78E/87E/135Q/150S/266N/267K/437G/486E/522I/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711Y/736M/750P/812E/830K/842S/871E/883H/894G/913R/932A, 24W/28S/29T/39Q/50E/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62V/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87S/135Q/150S/266N/267K/437G/486E/522V/569T/670T/692G/711H/736M/750P/812E/830K/842S/871E/883H/894G/913R/932A, 24W/28S/29T/39Q/50V/62N/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87H/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486L/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913E/932A, 24W/28S/29T/39Q/50V/62L/78E/87E/135Q/150S/266N/267K/437G/486E/522V/569T/670T/692G/711R/736M/750P/812E/830K/842S/871E/883H/894G/913R/932A, 24W/28S/29T/39G/50V/62L/78E/87E/135Q/150S/266N/267K/437G/486E/522V/569T/670T/692G/711H/736M/750P/812E/830K/842S/871E/883H/894G/913R/932A, 24R/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266D/267K/437G/486E/522V/569T/670T/692G/711H/736M/750P/812E/830K/842S/871E/883H/894G/913R/932A, 24W/28S/29T/39T/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50C/62L/78E/87E/135Q/150S/266N/267K/437G/486E/522V/569T/670T/692G/711H/736M/750P/812E/830K/842S/871E/883H/894G/913R/932A, 24A/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87K/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/812E/830K/842S/871E/883H/894G/913R/932A, 24W/28S/29E/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78L/87E/135Q/150S/266N/267K/437G/486E/522V/569T/670T/692G/711H/736M/750P/812E/830K/842S/871E/883H/894G/913R/932A, 24P/28S/29T/39Q/50V/62L/78E/87E/135Q/150S/266N/267K/437G/486E/522V/569T/670T/692G/711H/736M/750P/812E/830K/842S/871E/883H/894G/913R/932A, 24D/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692K/711H/736M/750P/812E/830K/842S/871E/883H/894G/913R/932A, 24W/28S/29D/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50Y/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486K/522V/569T/670T/692G/711H/736M/750P/812E/830K/842S/871E/883H/894G/913R/932A, 24W/28F/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569S/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883Q/894G/913R/932A, 24W/28S/29T/39Q/50F/62L/78E/87E/135Q/150S/266N/267K/437G/486E/522V/569T/670T/692G/711H/736M/750P/812E/830K/842S/871E/883H/894G/913R/932A, 24W/28W/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267V/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830E/842S/871E/883H/894G/913R/932A, 24W/28S/29T/39Q/50V/62E/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569I/670T/692G/711H/736M/750P/812E/830K/842S/871E/883H/894G/913R/932A, 24W/28V/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486V/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871V/883H/894G/913R/932A, 24W/28S/29S/39Q/50V/62L/78E/87E/135Q/150S/266N/267K/437G/486E/522V/569T/670T/692G/711H/736M/750P/812E/830K/842S/871E/883H/894G/913R/932A, 24W/28C/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830L/842S/871E/883H/894G/913R/932A, 24W/28K/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871N/883H/894G/913R/932A, 24W/28S/29T/39Q/50L/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932Q, 24W/28S/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711F/736M/750P/812E/830K/842S/871E/883H/894G/913R/932A, 24W/28S/29T/39Q/50V/62L/78E/87E/135Q/150S/266N/267K/437G/486E/522V/569E/670T/692G/711H/736M/750P/812E/830K/842S/871E/883H/894G/913R/932A, 24W/28S/29T/39Q/50V/62L/78F/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522R/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711E/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842F/871E/883H/894G/913R/932A, 24W/28S/29T/39Q/50N/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135D/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/437G/486E/522V/569T/670T/692G/711H/736 M/750P/812E/830K/842S/871E/883H/894G/913R/932A, 24W/28S/29T/39Q/50V/62L/78S/87E/135Q/150S/266N/267K/437G/486E/522V/569T/670T/692G/711H/736M/750P/812E/830K/842S/871E/883H/894G/913R/932A, 24V/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913S/932A, 24W/28S/29T/39Q/50V/62L/78M/87E/135Q/150S/266N/267K/437G/486E/522V/569T/670T/692G/711H/736M/750P/812E/830K/842S/871E/883H/894G/913R/932A, 24W/28S/29T/39Q/50R/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670Q/692G/711H/736M/750P/812E/830K/842S/871E/883H/894G/913R/932A, 24W/28S/29T/39Q/50V/62T/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894M/913R/932A, 24W/28S/29T/39Q/50K/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78N/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750L/812E/830K/842S/871E/883H/894G/913R/932A, 24W/28S/29T/39Q/50V/62L/78E/87E/135Q/150S/266N/267K/437G/486E/522V/569T/670T/692G/711H/736M/750R/812E/830K/842S/871E/883H/894G/913R/932A, 24W/28S/29T/39Q/50V/62L/78E/87E/135Q/150S/266N/267K/437G/486E/522V/569D/670T/692G/711H/736M/750P/812E/830K/842S/871E/883H/894G/913R/932A, 24I/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486N/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894V/913R/932A, 24W/28S/29T/39Q/50V/62L/78E/87E/135Q/150S/266N/267K/437G/486E/522V/569T/670T/692G/711H/736M/750P/812E/830D/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894W/913R/932A, 24W/28S/29T/39Q/50V/62L/78V/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522A/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670M/692G/711H/736M/750P/812E/830K/842S/871E/883H/894G/913R/932A, 24W/28S/29T/39Q/50V/62Y/78E/87E/135Q/150S/266N/267K/437G/486E/522V/569T/670T/692G/711H/736M/750P/812E/830K/842S/871E/883H/894G/913R/932A, 24W/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750K/812E/830K/842S/871E/883H/894G/913R/932A, 24W/28S/29T/39Q/50V/62L/78A/87E/135Q/150S/266N/267K/437G/486E/522V/569T/670T/692G/711H/736M/750P/812E/830K/842S/871E/883H/894G/913R/932A, 24W/28S/29T/39Q/50G/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894K/913R/932A, 24W/28S/29T/39Q/50V/62L/78E/87E/135Q/150S/266N/267K/437S/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692E/711H/736M/750P/812E/830K/842S/871E/883H/894G/913R/932A, 24W/28S/29T/39Q/50V/62L/78E/87E/135Q/150S/266N/267K/437G/486E/522V/569P/670T/692G/711H/736M/750P/812E/830K/842S/871E/883H/894G/913R/932A, 24W/28S/29T/39Q/50V/62L/78E/87E/135Q/150S/266N/267K/437G/486E/522V/569N/670T/692G/711H/736M/750P/812E/830K/842S/871E/883H/894G/913R/932A, 24W/28S/29T/39Q/50V/62L/78E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135N/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522Y/569T/670T/692G/711H/736M/750P/812E/830K/842S/871E/883H/894G/913R/932A, 24W/28S/29T/39Q/50V/62L/78E/87E/135Q/150S/266N/267K/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736F/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932T, 24G/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842K/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932W, 24W/28S/29T/39Q/50V/62L/78E/87E/135Q/150S/266N/267K/437G/486E/522V/569T/670T/692Q/711H/736M/750P/812E/830K/842S/871E/883H/894G/913R/932A, 24W/28S/29T/39Q/50V/62L/78E/87E/135F/150S/266N/267K/437G/486E/522V/569T/670T/692G/711H/736M/750P/812E/830K/842S/871E/883H/894G/913R/932A, 24W/28S/29T/39Q/50V/62L/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871F/883H/894G/913R/932A, 24W/28S/29T/39Q/50V/62L/78E/87E/135Q/150S/266N/267K/437G/486E/522V/569T/670Y/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692L/711H/736M/750P/812E/830K/842S/871E/883H/894G/913R/932A, 24W/28S/29T/39Q/50V/62L/78E/87E/135E/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670F/692G/711H/736M/750P/812E/830K/842S/871E/883H/894G/913R/932A, 24W/28S/29T/39Q/50H/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87I/135Q/150S/266N/267K/437G/486E/522V/569T/670T/692G/711H/736M/750P/812E/830K/842S/871E/883H/894G/913R/932A, 24W/28P/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883F/894G/913R/932A, 24W/28S/29T/39Q/50V/62L/78E/87E/135Q/150S/266N/267K/437H/486E/522V/569T/670T/692G/711H/736M/750P/812E/830K/842S/871E/883H/894G/913R/932A, 24W/28S/29T/39Q/50V/62I/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830W/842S/871E/883H/894G/913R/932A, 24W/28S/29T/39Q/50V/62L/78E/87E/135Q/150S/266N/267K/437G/486E/522V/569T/670T/692S/711H/736M/750P/812E/830K/842S/871E/883H/894G/913R/932A, 24W/28S/29K/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670E/692G/711H/736M/750P/812E/830K/842S/871E/883H/894G/913R/932A, 24W/28S/29T/39Q/50V/62L/78E/87E/135Q/150S/266N/267K/437G/486R/522V/569T/670T/692G/711H/736M/750P/812E/830K/842S/871E/883H/894G/913R/932A, 24W/28S/29T/39Q/50V/62L/78E/87E/135A/150S/266N/267K/437G/486E/522V/569T/670T/692G/711H/736M/750P/812E/830K/842S/871E/883H/894G/913R/932A, 24W/28S/29T/39I/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87N/135Q/150S/266N/267K/437G/486E/522V/569T/670T/692G/711H/736M/750P/812E/830K/842S/871E/883H/894G/913R/932A, 24W/28S/29T/39Q/50V/62L/78E/87M/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135C/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932L, 24W/28S/29T/39Q/50V/62L/78E/87E/135Q/150S/266N/267K/437G/486E/522V/569T/670G/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830F/842S/871E/883H/894G/913R/932A, 24W/28S/29T/39Q/50V/62L/78E/87E/135Q/150S/266N/267K/437G/486E/522H/569T/670T/692G/711H/736M/750P/812E/830K/842S/871E/883H/894G/913R/932A, 24W/28D/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522Q/569T/670T/692G/711H/736M/750P/812E/830K/842S/871E/883H/894G/913R/932A, 24W/28S/29T/39Q/50A/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670S/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830M/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842A/871E/883H/894G/913R/932A, 24W/28S/29T/39Q/50V/62L/78E/87T/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522T/569T/670T/692G/711H/736M/750P/812E/830K/842S/871E/883H/894G/913R/932A, 24K/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871K/883H/894G/913R/932A, 24W/28S/29T/39Q/50V/62L/78E/87E/135Q/150S/266N/267K/437G/486E/522V/569T/670T/692G/711H/736M/750E/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830T/842S/871E/883H/894G/913R/932A, 24W/28S/29T/39Q/50I/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932E, 24W/28S/29T/39Q/50V/62L/78E/87E/135Q/150S/266Q/267K/437G/486E/522V/569T/670T/692G/711H/736M/750P/812E/830K/842S/871E/883H/894G/913R/932A, 24W/28S/29T/39A/50V/62L/78E/87E/135Q/150S/266N/267K/437G/486E/522V/569T/670T/692G/711H/736M/750P/812E/830K/842S/871E/883H/894G/913R/932A, 24T/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871T/883H/894G/913R/932A, 24W/28S/29T/39Q/50V/62L/78E/87E/135Q/150S/266N/267K/437G/486E/522V/569T/670T/692G/711H/736M/750P/812E/830K/842M/871E/883H/894G/913R/932A, 24W/28S/29T/39Q/50V/62L/78E/87E/135Q/150S/266N/267K/437G/486E/522V/569T/670T/692G/711T/736M/750P/812E/830K/842S/871E/883H/894G/913R/932A, 24W/28S/29T/39Q/50V/62L/78E/87E/135Q/150S/266K/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486F/522V/569T/670T/692G/711H/736M/750P/812E/830K/842S/871E/883H/894G/913R/932A, 24W/28S/29T/39Q/50M/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486Y/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670R/692G/711H/736M/750P/812E/830K/842S/871E/883H/894G/913R/932A, 24M/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62F/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569Y/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711Q/736M/750P/812E/830K/842S/871E/883H/894G/913R/932A, 24W/28S/29T/39Q/50V/62L/78E/87E/135Q/150S/266N/267K/437G/486I/522V/569T/670T/692G/711H/736M/750P/812E/830K/842S/871E/883H/894G/913R/932A, 24W/28S/29T/39Q/50V/62L/78E/87E/135H/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670H/692G/711H/736M/750P/812E/830K/842S/871E/883H/894G/913R/932A, 24W/28S/29T/39Q/50V/62L/78E/87E/135Q/150S/266N/267K/437G/486E/522N/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894E/913R/932A, 24W/28S/29T/39Q/50V/62L/78E/87E/135Q/150S/266N/267K/437G/486E/522V/569T/670T/692G/711H/736M/750P/812E/830K/842S/871Q/883H/894G/913R/932A, 24W/28E/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842N/871E/883H/894G/913R/932A, 24W/28S/29T/39Q/50V/62L/78H/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913I/932A, 24W/28S/29T/39Q/50V/62L/78E/87E/135Q/150S/266N/267K/437G/486E/522V/569T/670T/692G/711H/736M/750P/812E/830K/842S/871E/883H/894G/913H/932A, 24W/28S/29T/39Q/50V/62L/78E/87E/135Q/150S/266N/267K/437G/486E/522V/569T/670T/692G/711H/736M/750P/812E/830K/842S/871Y/883H/894G/913R/932A, 24W/28S/29N/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670K/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692A/711H/736M/750P/812E/830K/842S/871E/883H/894G/913R/932A, 24W/28S/29T/39Q/50T/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842H/871E/883H/894G/913R/932A, 24W/28S/29T/39Q/50V/62L/78E/87E/135Q/150S/266N/267K/437G/486E/522V/569T/670T/692M/711H/736M/750P/812E/830K/842S/871E/883H/894G/913R/932A, 24W/28S/29Q/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894D/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913F/932A, 24W/28S/29T/39Q/50V/62L/78E/87E/135Q/150S/266N/267K/437G/486E/522V/569T/670D/692G/711H/736M/750P/812E/830K/842S/871E/883H/894G/913R/932A, 24W/28S/29T/39Q/50V/62M/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486C/522V/569T/670T/692G/711H/736M/750P/812E/830K/842S/871E/883H/894G/913R/932A, 24W/28S/29T/39Q/50V/62L/78T/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/830K/842S/871E/883H/894G/913R/932A, 24W/28S/29T/39Q/50V/62L/78E/87E/135Q/150S/266N/267H/437G/486E/522V/569T/670T/692G/711H/736M/750P/812E/830K/842S/871E/883H/894G/913R/932A, 24W/28S/29I/39Q/50V/62L/78E/87E/135Q/150S/266N/267K/437G/486E/522V/569T/670T/692G/711H/736M/750P/812E/830K/842S/871E/883H/894G/913R/932A, 24H/28S/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/1351/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692H/711H/736M/750P/812E/830K/842S/871E/883H/894G/913R/932A, 24W/28H/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913N/932A, 24W/28S/29T/39Q/50V/62L/78E/87E/135Q/150S/266N/267K/437G/486E/522V/569T/670T/692G/711H/736M/750P/812E/830K/842D/871E/883H/894G/913R/932A, 24W/28S/29T/39Q/50V/62L/78E/87E/135Q/150S/266N/267K/437G/486E/522K/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894H/913R/932A, 24W/28S/29T/39Q/50V/62L/78K/87E/135Q/150S/266N/267K/437G/486E/522V/569T/670T/692G/711H/736M/750P/812E/830K/842S/871E/883H/894G/913R/932A, 24W/28S/29T/39F/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871W/883H/894G/913R/932A, 24W/28S/29T/39Q/50V/62L/78E/87E/135Y/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842Q/871E/883H/894G/913R/932A, 24W/28S/29H/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78Q/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522D/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932Y, 24W/28S/29V/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842L/871E/883H/894G/913R/932A, 24W/28S/29T/39Q/50V/62L/78E/87E/135Q/150S/266N/267K/437G/486E/522V/569T/670T/692F/711H/736M/750P/812E/830K/842S/871E/883H/894G/913R/932A, 24W/28S/29R/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/8941/913R/932A, 24W/28S/29T/39N/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/711H/736M/750P/812E/830K/842S/871E/883H/894G/913R/932A, 24W/28S/29T/39Q/50V/62L/78E/87E/135Q/150S/266E/267K/437G/486E/522V/569T/670T/692G/711H/736M/750P/812E/830K/842S/871E/883H/894G/913R/932A, 24W/28S/29T/39Q/50V/62L/78C/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932P, 24W/28S/29T/39Q/50V/62L/78E/87E/135Q/150S/266N/267K/437G/486E/522V/569T/670T/692G/711H/736M/750P/812E/830K/842S/871E/883H/894G/913R/932D, 24W/28S/29T/39Q/50V/62L/78E/87E/135Q/150S/266N/267K/437G/486E/522V/569T/670T/692G/711H/736M/750P/812E/830Y/842S/871E/883H/894G/913R/932A, 24W/28S/29T/39Q/50V/62L/78E/87E/135K/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692D/711H/736M/750P/812E/830K/842S/871E/883H/894G/913R/932A, 24W/28G/29T/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711N/736M/750P/812E/830K/842S/871E/883H/894G/913R/932A, 24W/28S/29T/39Q/50V/62L/78E/87E/135Q/150S/266N/267K/437G/486W/522V/569T/670T/692G/711H/736M/750P/812E/830K/842S/871E/883H/894G/913R/932A, 24W/28S/29T/39Q/50D/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486A/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894N/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932G, 24W/28S/29T/39Q/50V/62L/78E/87E/135Q/150S/266N/267K/437G/486E/522V/569T/670T/692G/711D/736M/750P/812E/830K/842S/871E/883H/894G/913R/932A, 24W/28S/29T/39Q/50V/62L/78E/87E/135Q/150S/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871H/883H/894G/913R/932A, 24W/28S/29T/39Q/50V/62L/78E/87E/135Q/150S/266N/267K/437G/486H/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913K/932A, 24W/28S/29T/39Q/50V/62L/78E/87E/135Q/150S/266N/267K/437G/486E/522V/569T/670T/692G/711H/736M/750P/812E/830K/842S/871M/883H/894G/913R/932A, 24W/28S/29T/39E/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932N, 24W/28S/29T/39Q/50V/62L/78E/87E/135Q/150S/266N/267K/437G/486E/522V/569T/670T/692G/711H/736M/750P/812E/830K/842S/871E/883H/894G/913R/932M, 24W/28S/29T/39Q/50V/62L/78E/87E/135Q/150S/266N/267K/437G/486E/522V/569T/670T/692G/711H/736M/750P/812E/830K/842S/871E/883H/894G/913M/932A, 24W/28S/29T/39Q/50V/62L/78E/87E/135Q/150S/266N/267K/437G/486E/522V/569W/670T/692G/711H/736M/750P/812E/830K/842S/871E/883H/894G/913R/932A, 24W/28S/29T/39Q/50V/62L/78E/87E/135Q/150S/266N/267K/437G/486S/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/7111/736M/750P/812E/830K/842S/871E/883H/894G/913R/932A, 24W/28S/29T/39Q/50V/62L/78E/87E/135Q/150S/266N/267K/437G/486E/522V/569T/670T/692G/711C/736M/750P/812E/830K/842S/871E/883H/894G/913R/932A, 24W/28S/29T/39Q/50W/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522C/569T/670T/692G/711H/736M/750P/812E/830K/842S/871E/883H/894G/913R/932A, 24W/28S/29T/39Q/50V/62L/78E/87E/135Q/150S/266N/267K/437G/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R, 24W/28S/29W/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812G/830K/842S/871E/883H/894G/913R/932A, 24W/28S/29T/39Q/50V/62L/78D/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871D/883H/894G/913R/932A, 24W/28S/29T/39Q/50V/62L/78E/87E/135Q/150S/266N/267K/437G/486E/522V/569T/670T/692I/711H/736M/750P/812E/830K/842S/871E/883H/894G/913R/932A, 24W/28S/29F/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522M/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670N/692G/711H/736M/750P/812E/830K/842S/871E/883H/894G/913R/932A, 24W/28S/29M/39Q/50V/62L/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486G/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932K, 24W/28S/29T/39Q/50V/62Q/78E/87E/135Q/150S/266N/267K/437G/486E/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830S/842S/871E/883H/894G/913R/932A, 24W/28S/29T/39Q/50V/62L/78E/87E/135Q/150S/266N/267K/437G/486E/522V/569T/670T/692G/711H/736M/750P/812S/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830G/842S/871E/883H/894G/913R/932A, 24W/28S/29T/39Q/50V/62L/78E/87E/135Q/150S/266N/267K/437G/486M/522V/569T/670T/692G/711H/736M/750P/812E/830K/842S/871E/883H/894G/913R/932A, 24W/28S/29T/39Q/50V/62L/78E/87E/135Q/150S/266N/267K/437G/486E/522V/569T/670T/692G/711K/736M/750P/812E/830K/842S/871E/883H/894G/913R/932A, 24W/28S/29T/39Q/50V/62L/78E/87E/135Q/150S/266N/267K/437G/486E/522V/569T/670T/692G/711H/736M/750P/812E/830K/842S/871E/883H/894G/913R/932H, 24W/28S/29P/39Q/50V/62L/78E/87E/135Q/1505/266N/267K/437G/486E/522V/569T/670T/692G/711H/736M/750P/812E/830K/8425/871E/883H/894G/913R/932A, 24W/28S/29T/39Q/50V/62L/78E/87E/135Q/1505/266N/267K/437G/486E/522V/569T/670T/692W/711H/736M/750P/812E/830K/8425/871E/883H/894G/913R/932A, 24W/28S/29Y/39Q/50V/62L/78E/87E/135Q/1505/266N/267K/437G/486E/522V/569T/670T/692G/711H/736M/750P/812E/830K/8425/871E/883H/894G/913R/932A, 24W/28S/29T/39Q/50V/62L/78E/87E/135Q/1505/266N/267K/437G/486E/522V/569T/670T/692C/711H/736M/750P/812E/830K/8425/871E/883H/894G/913R/932A, 24W/28S/29G/39Q/50V/62L/78E/87E/135Q/1505/266N/267K/437G/486E/522V/569T/670T/692G/711H/736M/750P/812E/830K/8425/871E/883H/894G/913R/932A, 24W/28S/29T/39Q/50V/62L/78E/87E/135Q/1505/266N/267K/437G/486E/522V/569T/670T/692G/711H/736M/750P/812D/830K/8425/871E/883H/894G/913R/932A, 24W/28S/29T/39Q/50V/62L/78E/87E/135Q/1505/266N/267K/437G/486E/522V/569T/670T/692G/711H/736M/750P/812E/830K/8425/871E/883H/894G/913R/932C, 24W/28S/29T/39Q/50V/62L/78E/87E/135Q/1505/266N/267K/437G/486E/522V/569T/670T/692G/711H/736M/750P/812E/830K/8425/871I/883H/894G/913R/932A, 24W/28S/29T/39Q/50V/62L/78E/87E/135Q/1505/266N/267K/437G/486E/522V/569T/670T/692Y/711H/736M/750P/812E/830K/8425/871E/883H/894G/913R/932A, 24W/28S/29T/39Q/50V/62L/78E/87E/135Q/1505/266N/267K/437G/486E/522V/569T/670T/692G/711H/736M/750Q/812E/830K/8425/871E/883H/894G/913R/932A, 24W/28S/29T/39Q/50V/62L/78E/87E/135Q/1505/266N/267K/437G/486E/522V/569T/670T/692G/711H/736M/750P/812E/830H/8425/871E/883H/894G/913R/932A, 24W/28S/29T/39Q/50V/62L/78E/87E/135Q/1505/266N/267K/437G/486E/522V/569T/670T/692G/711H/736M/750P/812E/830K/842C/871E/883H/894G/913R/932A, 24W/28S/29T/39Q/50V/62L/78E/87E/135Q/1505/266N/267K/437G/486E/522V/569T/670T/692G/711H/736M/750P/812E/830K/8425/871S/883H/894G/913R/932A, 24W/28S/29T/39Q/50V/62L/78E/87E/135Q/150S/266N/267K/437G/486E/522V/569T/670T/692G/711H/736M/750P/812E/830K/8425/871C/883H/894G/913R/932A, or 24W/28S/29T/39Q/50V/62L/78E/87E/135Q/150S/266N/267K/437G/486E/522V/569T/670T/692G/711H/736M/750P/812E/830K/842S/871E/883H/894G/913W/932A, wherein the amino acid positions are relative to the reference sequence corresponding to residues 20-944 of SEQ ID NO: 2, or relative to the reference sequence corresponding to SEQ ID NO: 2.

In some embodiments, the amino acid sequence of the engineered acid alpha-glucosidase comprises a sequence comprising residues 20-944 of an even-numbered SEQ ID NO. of SEQ ID NOs: 14-754, or a sequence comprising an even-numbered SEQ ID NO. of SEQ ID NOs: 14-754.

In some embodiments, the amino acid sequence of the engineered acid alpha-glucosidase comprises residues 20-944 of SEQ ID NO: 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, or 754. In some embodiments, the amino acid sequence optionally has 1, 2, 3, 4, 5, 6, 7, 8, 9, or up to 10 amino acid insertions, deletions, or substitutions. In some embodiments, the amino acid sequence optionally has 1, 2, 3, 4, 5, 6, 7, 8, 9, or up to 10 amino acid substitutions. In some embodiments, the amino acid sequence optionally has 1, 2, 3, 4, up to 5 amino acid insertions, deletions, or substitutions. In some embodiments, the amino acid sequence optionally has 1, 2, 3, 4, up to 5 amino acid substitutions. In some embodiments, the amino acid substitutions comprise non-conservative or conservative substitutions. In some embodiments, the amino acid substitutions comprise conservative substitutions. In some embodiments, guidance on non-conservative and conservative substitutions are provided by the variants and Examples disclosed herein.

In some embodiments, the amino acid sequence of the engineered acid alpha-glucosidase comprises SEQ ID NO: 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, or 754. In some embodiments, the amino acid sequence optionally has 1, 2, 3, 4, 5, 6, 7, 8, 9, or up to 10 amino acid insertions, deletions, or substitutions. In some embodiments, the amino acid sequence optionally has 1, 2, 3, 4, 5, 6, 7, 8, 9, or up to 10 amino acid substitutions. In some embodiments, the amino acid sequence optionally has 1, 2, 3, 4, up to 5 amino acid insertions, deletions, or substitutions. In some embodiments, the amino acid sequence optionally has 1, 2, 3, 4, up to 5 amino acid substitutions. In some embodiments, the amino acid substitutions comprises non-conservative or conservative substitutions. In some embodiments, the amino acid substitutions comprise conservative substitutions. In some embodiments, guidance on non-conservative and conservative substitutions are provided by the variants and Examples disclosed herein.

In some embodiments, the engineered acid alpha-glucosidase of the present disclosure has acid alpha-glucosidase activity. In some embodiments, the engineered acid alpha-glucosidase has acid alpha-glucosidase activity and at least one improved or enhanced property as compared to a reference acid alpha-glucosidase.

In some embodiments, the engineered acid alpha-glucosidase is more thermostable than the reference acid alpha-glucosidase.

In some embodiments, the engineered acid alpha-glucosidase is more stable at pH 7 (e.g., at neutral pH) than the reference acid alpha-glucosidase.

In some embodiments, the engineered acid alpha-glucosidase is more stable at acid pi, particularly about pH 4.4, than the reference acid alpha-glucosidase.

In some embodiments, the engineered acid alpha-glucosidase exhibits increased expression than the reference acid alpha-glucosidase.

In some embodiments, the engineered acid alpha-glucosidase is more stable in lysosomes than the reference acid alpha-glucosidase.

In some embodiments, the engineered acid alpha-glucosidase is more stable in plasma, particularly human plasma, than the reference acid alpha-glucosidase.

In some embodiments, the engineered acid alpha-glucosidase is more readily taken up human cells, than the reference acid alpha-glucosidase.

In some embodiments, the engineered acid alpha-glucosidase exhibits greater enzymatic activity in cell lysates than the reference acid alpha-glucosidase.

In some embodiments, the engineered acid alpha-glucosidase exhibits reduced immunogenicity as compared to the reference acid alpha-glucosidase.

In some embodiments, the reference acid alpha-glucosidase has a sequence corresponding to residues 20-944 of SEQ ID NO: 2 or 12, or a sequence corresponding to SEQ ID NO: 2 or 12. In some embodiments, the reference acid alpha-glucosidase has the sequence corresponding to residues 20-944 of SEQ ID NO: 2, or the sequence corresponding to SEQ ID NO: 2. In some embodiments, the reference acid alpha-glucosidase has the sequence corresponding to residues 20-944 of SEQ ID NO: 12, or the sequence corresponding to SEQ ID NO: 12. Exemplary improved properties are provided in the Examples.

In some embodiments, the engineered acid alpha-glucosidase exhibits at least one improved property selected from: i) enhanced catalytic activity; ii) increased tolerance to pH 7; iii) increased tolerance to pH 4.4; iv) increased stability in lysosomes; v) increased expression; vi) increased uptake into cells; vii) increased enzymatic activity in cell lysates; viii) increased stability in plasma/serum; and ix) reduced immunogenicity; or a combination of any of i), ii), iii), iv), v), vi), vii), viii), and ix) as compared to a reference acid alpha-glucosidase. In some embodiments, the reference acid alpha-glucosidase has the sequence corresponding to residues 20-944 of SEQ ID NO: 2 or 12, or the sequence corresponding to SEQ ID NO: 2 or 12. In some embodiments, the reference acid alpha-glucosidase has the sequence corresponding to residues 20-944 of SEQ ID NO: 12, or the sequence corresponding to SEQ ID NO: 12.

In some embodiments, the engineered acid alpha-glucosidase exhibits reduced immunogenicity as compared to the reference acid alpha-glucosidase having a sequence corresponding to residues 20-944 of SEQ ID NO: 2 or 12, or a sequence corresponding to SEQ ID NO: 2 or 12. In some embodiments, the engineered acid alpha-glucosidase exhibits a reduction in Total Immunogenic Score (TIS) of greater than 10, greater than 100, or greater than 200 as compared to the reference acid alpha-glucosidase of SEQ ID NO: 2. In some embodiments, the engineered acid alpha-glucosidase exhibits a reduction in Immunogenic Hit Count (IHC) of greater than 2, greater than 5, or greater than 20 as compared to the reference acid alpha-glucosidase of SEQ ID NO: 2. In some embodiments, the engineered acid alpha-glucosidase exhibits a reduction in Total Immunogenic Score (TIS) of greater than 10, greater than 100, or greater than 200 as compared to the reference acid alpha-glucosidase of SEQ ID NO: 12. In some embodiments, the engineered acid alpha-glucosidase exhibits a reduction in Immunogenic Hit Count (IHC) of greater than 2, greater than 5, or greater than 20 as compared to the reference acid alpha-glucosidase of SEQ ID NO: 12. Exemplary engineered acid alpha-glucosidase polypeptides exhibiting reduced immunogenicity based on TIS and/or IHC can be selected from the engineered acid alpha-glucosidase presented in Table 4-1 of the Examples.

In some embodiments, the engineered acid alpha-glucosidase exhibits lower numbers of sequence regions presented by antigen-presenting cells and/or reduced frequency of peptide presentation as compared to a reference acid alpha-glucosidase polypeptide, in particular the reference acid alpha-glucosidase polypeptide corresponding to SEQ ID NO: 2, in a MHC associated peptide proteomics (MAPPs) assay, as provided in the Examples.

In some embodiments, the engineered acid alpha-glucosidase described herein comprises a pre-pro-polypeptide of the engineered acid alpha-glucosidase. In some embodiments, the pre-pro-polypeptide of the engineered acid alpha-glucosidase comprises a eukaryotic or synthetic signal peptide sequence. In some embodiments, the signal peptide of the pre-pro-polypeptide of the engineered acid alpha-glucosidase comprises a mouse or human signal peptide sequence. In some embodiments, the signal peptide comprises a sequence comprising residues 1-19 of SEQ ID NO: 2 or 12.

In some embodiments, the engineered acid alpha-glucosidase comprises a pro-polypeptide of the engineered acid alpha-glucosidase described herein. In some embodiments, the pro-polypeptide of the engineered acid alpha-glucosidase lacks a signal sequence. In some embodiments, the pro-polypeptide of the engineered acid alpha-glucosidase comprises residues 20-944 of an engineered acid alpha-glucosidase described herein.

In some embodiments, the engineered acid alpha-glucosidase is a mature form of an engineered acid alpha-glucosidase polypeptide described herein. In some embodiments, the mature form of the engineered acid alpha-glucosidase is a secreted form of the engineered acid alpha-glucosidase.

In some embodiments, the engineered acid alpha-glucosidase further comprises a fusion polypeptide. In some embodiments, the engineered acid alpha-glucosidase polypeptide described herein can be fused to a variety of polypeptide sequences, such as, by way of example and not limitation, polypeptide tags that can be used for detection and/or purification. In some embodiments, the fusion protein of the engineered acid alpha-glucosidase polypeptides comprises a glycine-histidine or histidine-tag (His-tag). In some embodiments, the fusion protein of the engineered acid alpha-glucosidase polypeptide comprises an epitope tag, such as c-myc, FLAG, V5, or hemagglutinin (HA). In some embodiments, the fusion protein of the engineered acid alpha-glucosidase polypeptide comprises a GST, SUMO, Strep, MBP, or GFP tag. In some embodiments, the fusion is to the amino (N-) terminus of an engineered acid alpha-glucosidase polypeptide. In some embodiments, the fusion is to the carboxy (C-) terminus of the engineered acid alpha-glucosidase polypeptide. In some embodiments, the fusion polypeptide is inserted following the signal sequence and before the engineered acid alpha-glucosidase polypeptide to allow expression and secretion of a polypeptide comprising the fusion polypeptide (e.g., polypeptide tag) and polypeptide.

In some embodiments, the engineered acid alpha-glucosidase is an isolated or purified preparation. In some embodiments, the engineered acid alpha-glucosidase is purified from a mixture (e.g., from cells or culture medium) using any one or more of the known techniques for protein purification, including, among others, lysozyme treatment, sonication, filtration, salting-out, ultra-centrifugation, and chromatography.

Chromatographic techniques for isolation and purification of the engineered acid alpha-glucosidase polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying a particular protein depends, in part, on factors such as net charge, hydrophobicity, hydrophilicity, molecular weight, molecular shape, etc., and will be apparent to those having skill in the art. In some embodiments, affinity techniques may be used to isolate the polypeptides. For affinity chromatography purification, any antibody that specifically binds acid alpha-glucosidase polypeptide of interest may find use. For the production of antibodies, various host animals, including but not limited to rabbits, mice, rats, camelids, etc., are immunized by injection with an engineered acid alpha-glucosidase polypeptide, or a fragment thereof.

In some embodiments, the present disclosure further provides functional fragments or biologically active fragments of an engineered acid alpha-glucosidase polypeptide described herein. Thus, for each and every embodiment herein of an engineered acid alpha-glucosidase, a functional fragment or biologically active fragment of the engineered acid alpha-glucosidase is provided herewith. In some embodiments, a functional fragment or biologically active fragments of an engineered acid alpha-glucosidase comprises at least about 90%, at least about 95%, at least about 96%, at least about 97%, at least about 98%, or at least about 99% of the activity of the acid alpha-glucosidase polypeptide from which it was derived (i.e., the parent acid alpha-glucosidase).

In some embodiments, a functional or biologically active fragment of an engineered acid alpha-glucosidase herein comprises at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the parent sequence of the engineered acid alpha-glucosidase. In some embodiments, functional fragment or biologically active fragment comprises at least 90%, 91%, 92%, 93%, 94%, 95%, 96%, 97%, 98%, or 99% of the parent sequence of the acid alpha-glucosidase. In some embodiments, the functional fragment will be truncated by less than 5, less than 10, less than 15, less than 10, less than 25, less than 30, less than 35, less than 40, less than 45, less than 50, less than 55, less than 60, less than 65, or less than 70 amino acids.

In some embodiments, the functional fragments or biologically active fragments of the engineered acid alpha-glucosidase polypeptide described herein include at least a substitution or substitution set in the amino acid sequence of the engineered acid alpha-glucosidase described herein. Accordingly, in some embodiments, the functional fragment(s) or biologically active fragment(s) of the engineered acid alpha-glucosidase displays the enhanced or improved property associated with the substitution or substitution set in the parent acid alpha-glucosidase.

### Polynucleotides Encoding Recombinant Polypeptides, Expression Vectors, and Host Cells

In another aspect, the present disclosure provides recombinant polynucleotides encoding the engineered acid alpha-glucosidase polypeptides described herein. In some embodiments, the polynucleotides are operably linked to one or more heterologous or homologous regulatory sequences that control gene expression to create a recombinant polynucleotide capable of expressing the polypeptide. Expression constructs containing a heterologous polynucleotide encoding the engineered acid alpha-glucosidase polypeptides can be introduced into appropriate host cells to express the corresponding engineered acid alpha-glucosidase polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the engineered acid alpha-glucosidase polypeptide. Thus, having knowledge of a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made encoding the engineered acid alpha-glucosidase polypeptides described herein by selecting combinations based on the possible codon choices, and all such variations are to be considered specifically disclosed for any polypeptide described herein, including the engineered acid alpha-glucosidase variants provided in the Examples.

In some embodiments, the recombinant polynucleotide is codon optimized. In other words, the codons are preferably selected to fit the host cell in which the protein is being produced. For example, preferred codons used in bacteria are used for expression in bacteria, preferred codons used in fungi are typically used for expression in fungi, and preferred codons used in mammals are used for expression in mammals and mammalian cells. In some embodiments, codon optimized polynucleotides encoding an engineered acid alpha-glucosidase polypeptide contains preferred codons at about 40%, 50%, 60%, 70%, 80%, or greater than 90% of codon positions of the full length coding region. In some embodiments, the present disclosure provides recombinant polynucleotide sequences in which the codons are optimized for expression in human cells or tissues.

As discussed above, it is to be understood that the present disclosure provides recombinant polynucleotides encoding each and every engineered acid alpha-glucosidase polypeptides described herein. Accordingly by way of example and not limitation, in some embodiments, a recombinant polynucleotide of the present disclosure comprises a polynucleotide sequence encoding an engineered acid alpha-glucosidase, or biologically active fragment thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-944 of an even numbered SEQ ID NO. of SEQ ID NOs: 2, 12, and 14-754, or to an even numbered SEQ ID NO. of SEQ ID NOs: 2, 12, and 14-754, wherein the amino acid sequence comprises one or more substitutions relative to the reference sequence corresponding to residues 20-944 of SEQ ID NO: 12 or 2, or relative to the reference sequence corresponding to SEQ ID NO: 12 or 2.

In some embodiments, the recombinant polynucleotide comprises a polynucleotide sequence encoding an engineered acid alpha-glucosidase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to residues 20-944 of SEQ ID NO: 12 or 2, or to the reference sequence corresponding to SEQ ID NO: 12 or 2, wherein the amino acid sequence comprises one or more substitutions relative to the reference sequence corresponding to residues 20-944 of SEQ ID NO: 12 or 2, or relative to the reference sequence corresponding to SEQ ID NO: 12 or 2.

In some embodiments, the recombinant polynucleotide comprises a polynucleotide sequence encoding an engineered acid alpha-glucosidase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-944 of SEQ ID NO: 12, or to the reference sequence corresponding to SEQ ID NO: 12, wherein the amino acid sequence comprises one or more substitutions relative to the reference sequence corresponding to residues 20-944 of SEQ ID NO: 12, or relative to the reference sequence corresponding to SEQ ID NO: 12.

In some embodiments, the recombinant polynucleotide comprises a polynucleotide sequence encoding an engineered acid alpha-glucosidase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-944 of an even numbered SEQ ID NO. of SEQ ID NOs: 14-754, or to a reference sequence corresponding to an even numbered SEQ ID NO. of SEQ ID NOs: 14-754, wherein the amino acid sequence comprises one or more substitutions relative to the reference sequence corresponding to residues 20-944 of SEQ ID NO: 12 or 2, or relative to the reference sequence corresponding to SEQ ID NO: 12 or 2.

In some embodiments, the recombinant polynucleotide comprises a polynucleotide sequence encoding an engineered acid alpha-glucosidase comprising an amino acid sequence comprising at least a substitution at amino acid position 24, 28, 29, 39, 50, 62, 78, 87, 135, 150, 266, 267, 305, 437, 486, 522, 569, 670, 692, 711, 736, 750, 812, 830, 842, 871, 883, 894, 913, or 932, or combinations thereof, wherein the amino acid positions are relative to the reference sequence corresponding to residues 20-944 of SEQ ID NO: 12, or relative to the reference sequence corresponding to SEQ ID NO: 12.

In some embodiments, the recombinant polynucleotide encodes an engineered acid alpha-glucosidase comprising an amino acid sequence comprising at least a substitution at amino acid position 305.

In some embodiments, the recombinant polynucleotide encodes an engineered acid alpha-glucosidase comprising an amino acid sequence comprising at least a substitution at amino acid position 24.

In some embodiments, the recombinant polynucleotide encodes an engineered acid alpha-glucosidase comprising an amino acid sequence comprising at least a substitution at amino acid position 28.

In some embodiments, the recombinant polynucleotide encodes an engineered acid alpha-glucosidase comprising an amino acid sequence comprising at least a substitution at amino acid position 29.

In some embodiments, the recombinant polynucleotide encodes an engineered acid alpha-glucosidase comprising an amino acid sequence comprising at least a substitution at amino acid position 39.

In some embodiments, the recombinant polynucleotide encodes an engineered acid alpha-glucosidase comprising an amino acid sequence comprising at least a substitution at amino acid position 50.

In some embodiments, the recombinant polynucleotide encodes an engineered acid alpha-glucosidase comprising an amino acid sequence comprising at least a substitution at amino acid position 62.

In some embodiments, the recombinant polynucleotide encodes an engineered acid alpha-glucosidase comprising an amino acid sequence comprising at least a substitution at amino acid position 78.

In some embodiments, the recombinant polynucleotide encodes an engineered acid alpha-glucosidase comprising an amino acid sequence comprising at least a substitution at amino acid position 87.

In some embodiments, the recombinant polynucleotide encodes an engineered acid alpha-glucosidase comprising an amino acid sequence comprising at least a substitution at amino acid position 135.

In some embodiments, the recombinant polynucleotide encodes an engineered acid alpha-glucosidase comprising an amino acid sequence comprising at least a substitution at amino acid position 150.

In some embodiments, the recombinant polynucleotide encodes an engineered acid alpha-glucosidase comprising an amino acid sequence comprising at least a substitution at amino acid position 266.

In some embodiments, the recombinant polynucleotide encodes an engineered acid alpha-glucosidase comprising an amino acid sequence comprising at least a substitution at amino acid position 267.

In some embodiments, the recombinant polynucleotide encodes an engineered acid alpha-glucosidase comprising an amino acid sequence comprising at least a substitution at amino acid position 437.

In some embodiments, the recombinant polynucleotide encodes an engineered acid alpha-glucosidase comprising an amino acid sequence comprising at least a substitution at amino acid position 486.

In some embodiments, the recombinant polynucleotide encodes an engineered acid alpha-glucosidase comprising an amino acid sequence comprising at least a substitution at amino acid position 522.

In some embodiments, the recombinant polynucleotide encodes an engineered acid alpha-glucosidase comprising an amino acid sequence comprising at least a substitution at amino acid position 569.

In some embodiments, the recombinant polynucleotide encodes an engineered acid alpha-glucosidase comprising an amino acid sequence comprising at least a substitution at amino acid position 670.

In some embodiments, the recombinant polynucleotide encodes an engineered acid alpha-glucosidase comprising an amino acid sequence comprising at least a substitution at amino acid position 692.

In some embodiments, the recombinant polynucleotide encodes an engineered acid alpha-glucosidase comprising an amino acid sequence comprising at least a substitution at amino acid position 711.

In some embodiments, the recombinant polynucleotide encodes an engineered acid alpha-glucosidase comprising an amino acid sequence comprising at least a substitution at amino acid position 736.

In some embodiments, the recombinant polynucleotide encodes an engineered acid alpha-glucosidase comprising an amino acid sequence comprising at least a substitution at amino acid position 750.

In some embodiments, the recombinant polynucleotide encodes an engineered acid alpha-glucosidase comprising an amino acid sequence comprising at least a substitution at amino acid position 812.

In some embodiments, the recombinant polynucleotide encodes an engineered acid alpha-glucosidase comprising an amino acid sequence comprising at least a substitution at amino acid position 830.

In some embodiments, the recombinant polynucleotide encodes an engineered acid alpha-glucosidase comprising an amino acid sequence comprising at least a substitution at amino acid position 842.

In some embodiments, the recombinant polynucleotide encodes an engineered acid alpha-glucosidase comprising an amino acid sequence comprising at least a substitution at amino acid position 305.

In some embodiments, the recombinant polynucleotide encodes an engineered acid alpha-glucosidase comprising an amino acid sequence comprising at least a substitution at amino acid position 883.

In some embodiments, the recombinant polynucleotide encodes an engineered acid alpha-glucosidase comprising an amino acid sequence comprising at least a substitution at amino acid position 894.

In some embodiments, the recombinant polynucleotide encodes an engineered acid alpha-glucosidase comprising an amino acid sequence comprising at least a substitution at amino acid position 913.

In some embodiments, the recombinant polynucleotide encodes an engineered acid alpha-glucosidase comprising an amino acid sequence comprising at least a substitution at amino acid position 932.

In some embodiments, the recombinant polynucleotide comprises a polynucleotide sequence encoding an engineered acid alpha-glucosidase comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to an engineered acid alpha-glucosidase with a substitution or substitution set as set forth in Table 3-1, wherein the amino acid positions are relative to the reference sequence corresponding to residues 20-944 of SEQ ID NO: 12 or 2, or relative to the reference sequence corresponding to SEQ ID NO: 12 or 2.

In some embodiments, the recombinant polynucleotide comprises a polynucleotide sequence encoding an engineered acid alpha-glucosidase comprising an amino acid sequence comprising at least one substitution as set forth in Table 3-1, wherein the amino acid positions are relative to the reference sequence corresponding to residues 20-944 of SEQ ID NO: 12 or 2, or relative to the reference sequence corresponding to SEQ ID NO: 12 or 2.

In some embodiments, the recombinant polynucleotide comprises a polynucleotide sequence encoding an engineered acid alpha-glucosidase comprising an amino acid sequence comprising at least a substitution set as set forth in Table 3-1, wherein the amino acid positions are relative to the reference sequence corresponding to residues 20-944 of SEQ ID NO: 12 or 2, or relative to the reference sequence corresponding to SEQ ID NO: 12 or 2.

In some embodiments, the recombinant polynucleotide comprises a polynucleotide sequence encoding an engineered acid alpha-glucosidase comprising an amino acid sequence comprising at least a substitution set at amino acid positions 24/28/29/39/50/62/78/87/135/150/266/267/305/437/486/522/569/670/692/711/736/750/812/830/842/871/883/894/913/932, 24/28/29/39/50/62/78/87/135/150/266/267/437/486/522/569/670/692/711/736/750/812/830/842/871/883/894/913/932, 24/28/29/39/50/62/78/87/135/150/266/267/437/486/522/569/670/692/711/736/750/812/830/871/883/894/913/932, 28/29/39/50/62/78/87/135/150/266/267/437/486/522/569/670/692/711/736/750/812/830/842/871/883/89 4/913/932, 24/28/29/39/50/78/87/135/150/266/267/437/486/522/569/670/692/711/736/750/812/830/842/871/883/89 4/913/932, 24/28/29/39/50/62/78/87/135/150/266/267/437/486/522/569/670/692/711/736/750/812/830/842/883/894/913/932, 24/28/29/39/50/62/78/87/135/150/266/267/437/486/522/670/692/711/736/750/812/830/842/871/883/894/913/932, 24/28/29/39/50/62/78/87/135/150/266/267/437/486/522/569/692/711/736/750/812/830/842/871/883/894/913/932, 24/28/29/39/50/62/78/87/135/150/266/267/437/486/522/569/670/692/736/750/812/830/842/871/883/894/913/932, 24/28/29/39/50/62/78/87/135/150/266/267/437/486/522/569/670/692/711/750/812/830/842/871/883/894/913/932, 24/28/29/39/50/62/78/87/135/150/266/267/437/486/569/670/692/711/736/750/812/830/842/871/883/894/913/932, 24/28/29/39/50/62/78/87/135/150/266/267/437/486/522/569/670/692/711/736/812/830/842/871/883/894/913/932, 24/28/39/50/62/78/87/135/150/266/267/437/486/522/569/670/692/711/736/750/812/830/842/871/883/89 4/913/932, 24/28/29/39/50/62/78/87/135/150/266/437/486/522/569/670/692/711/736/750/812/830/842/871/883/894/913/932, 24/29/39/50/62/78/87/135/150/266/267/437/486/522/569/670/692/711/736/750/812/830/842/871/883/89 4/913/932, 24/28/29/39/50/62/78/135/150/266/267/437/486/522/569/670/692/711/736/750/812/830/842/871/883/89 4/913/932, 24/28/29/39/50/62/78/87/135/150/266/267/486/522/569/670/692/711/736/750/812/830/842/871/883/894/913/932, 24/28/29/39/50/62/87/135/150/266/267/437/486/522/569/670/692/711/736/750/812/830/842/871/883/89 4/913/932, 24/28/29/39/50/62/78/87/135/150/266/267/437/486/522/569/670/692/711/736/750/812/830/8425/871/88 3/894/913/932, 24/28/29/39/50/62/78/87/135/266/267/437/486/522/569/670/692/711/736/750/812/830/842/871/883/894/913/932, 24/28/29/39/50/62/78/87/135/150/266/267/437/486/522/569/670/692/711/736/750/812/842/871/883/894/913/932, 24/28/29/39/50/62/78/87/135/150/266/267/437/486/522/569/670/692/711/736/750/830/842/871/883/894/913/932, 24/28/29/39/62/78/87/135/150/266/267/437/486/522/569/670/692/711/736/750/812/830/842/871/883/89 4/913/932, 24/28/29/39/50/62/78/87/135/150/266/267/437/486/522/569/670/711/736/750/812/830/842/871/883/894/913/932, 24/28/29/39/50/62/78/87/135/150/266/267/437/486/522/569/670/692/711/736/750/812/830/842/871/883/913/932, 24/28/29/39/50/62/78/87/135/150/267/437/486/522/569/670/692/711/736/750/812/830/842/871/883/894/913/932, 24/28/29/39/50/62/78/87/135/150/266/267/437/522/569/670/692/711/736/750/812/830/842/871/883/894/913/932, 24/28/29/39/50/62/78/87/135/150/266/267/437/486/522/569/670/692/711/736/750/812/830/842/871/883/894/913, or 24/28/29/39/50/62/78/87/135/150/266/267/437/486/522/569/670/692/711/736/750/8125/830/842/871/88 3/894/913/932, wherein the positions are relative to SEQ ID NO: 2.

In some embodiments, the recombinant polynucleotide comprises a polynucleotide sequence encoding an engineered acid alpha-glucosidase comprising an amino acid sequence comprising residues 20-944 of an even-numbered SEQ ID NO. of SEQ ID NOs: 14-754, or comprising an even-numbered SEQ ID NO. of SEQ ID NOs: 14-754.

In some embodiments, the recombinant polynucleotide comprises a polynucleotide sequence encoding an engineered acid alpha-glucosidase comprising an amino acid sequence comprising residues 20-944 of SEQ ID NO: 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, or 754.

In some embodiments, the recombinant polynucleotide comprises a polynucleotide sequence encoding an engineered acid alpha-glucosidase comprising an amino acid sequence comprising SEQ ID NO: 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, or 754.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference polynucleotide sequence corresponding to nucleotide residues 58-2832 of an odd-numbered SEQ ID NO. of SEQ ID NOs: 13-753, or to a reference polynucleotide sequence corresponding to an odd-numbered SEQ ID NO. of SEQ ID NOs: 13-753, wherein the recombinant polynucleotide encodes an acid alpha-glucosidase.

In some embodiments, the recombinant polynucleotide comprises a polynucleotide sequence comprising nucleotide residues 58-2832 of an odd-numbered SEQ ID NO. of SEQ ID NOs: 13-753, or a polynucleotide sequence comprising an odd-numbered SEQ ID NO. of SEQ ID NOs: 13-753.

In some embodiments, the recombinant polynucleotide comprises a polynucleotide sequence comprising nucleotide residues 58-2832 of SEQ ID NO: 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, or 753.

In some embodiments, the recombinant polynucleotide comprises a polynucleotide sequence comprising SEQ ID NO: 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, or 753.

In some embodiments, the recombinant polynucleotide encoding any of the engineered acid alpha-glucosidase polypeptides provided herein is manipulated in a variety of ways to provide for expression of the encoded polypeptide. In some embodiments, the polynucleotides encoding the polypeptides are provided as expression vectors where one or more control sequences is present to regulate the expression of the polynucleotides and/or polypeptides. In some embodiments, the control sequences include among other sequences, promoters, Kozak sequence, leader sequences, polyadenylation sequences, pro-peptide sequences, signal peptide sequences, DNA based regulatory elements for gene therapy retention and transcription terminators. Manipulation of the isolated polynucleotide prior to its insertion into a vector may be desirable or necessary depending on the expression vector. The techniques for modifying polynucleotides and nucleic acid sequences utilizing recombinant DNA methods are well-known in the art.

In some embodiments, suitable promoters can be selected based on the host cells used for expression. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (see, e.g., Villa-Kamaroff et al., Proc. Natl Acad. Sci. USA, 1978, 75:3727-3731), as well as the tac promoter (see, e.g., DeBoer et al., Proc. Natl Acad. Sci. USA, 1983, 80:21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (see, e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (see, e.g., Romanos et al., Yeast, 1992, 8:423-488). Exemplary promoters for use in mammalian cells include, but are not limited to those from cytomegalovirus (CMV), chicken β-actin promoter fused with the CMV enhancer, Simian virus 40 (SV40), from Homo sapiens phosphoglycerate kinase, beta actin, elongation factor-1a or glyceraldehyde-3-phosphate dehydrogenase, or from Gallus β-actin.

In some embodiments, the control sequence is a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice finds use in the present invention. For bacterial expression, the transcription terminators can be a Rho-dependent terminators that rely on a Rho transcription factor, or a Rho-independent, or intrinsic terminators, which do not require a transcription factor. Exemplary bacterial transcription terminators are described in Peters et al., J Mol Biol., 2011, 412(5):793-813. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra). Exemplary terminators for mammalian cells include, but are not limited to, those from cytomegalovirus (CMV), Simian vacuolating virus 40 (SV40), from Homo sapiens growth hormone hGH, from bovine growth hormone BGH, and from human or rabbit beta globulin.

In some embodiments, the control sequence is a suitable leader sequence, 5′-cap modification, 5′ UTR, etc. In some embodiments, these regulatory sequence elements mediate binding to molecules involved in mRNA trafficking and translation, inhibit 5′-exonucleolytic degradation and confer resistance to de-capping. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence comprises a 3′ untranslated nucleic acid region and polyadenylation tail nucleic acid sequence, sequences operably linked to the 3′ terminus of the protein coding nucleic acid sequence which mediate binding to proteins involved in mRNA trafficking and translation and mRNA half-life. Any polyadenylation sequence and 3′ UTR which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Biol., 1995, 15:5983-5990). Useful polyadenylation and 3′ UTR sequences for mammalian host cells include, but are not limited to the 3′-UTRs of α- and β-globin mRNAs that harbor several sequence elements that increase the stability and translation of mRNA.

In some embodiments, the control sequence is a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region that directs the expressed polypeptide into the secretory pathway of a host cell of choice finds use for expression of the engineered acid alpha-glucosidase polypeptides provided herein. Effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for mammalian host cells include but are not limited to those from the genes for immunoglobulin gamma (IgG). In some embodiments, the signal peptide comprises the naturally occurring signal sequence in human acid alpha-glucosidase. In some embodiments, the encoded signal peptide comprises a sequence comprising residues 1-19 of SEQ ID NO: 2 or 12.

In some embodiments, the control sequence comprises one or more regulatory sequences that facilitate regulation of the expression of the polynucleotide and/or corresponding encoded polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter. Exemplary inducible promoters regulated by exogenous agents include the zinc-inducible sheep metallothionine (MT) promoter, dexamethasone (Dex)-inducible promoter, mouse mammary tumor virus (MMTV) promoter; ecdysone insect promoter, tetracycline-inducible promoter system, RU486-inducible promoter system, and the rapamycin-inducible promoter system.

In some embodiments, the recombinant expression vector may be any suitable vector (e.g., a plasmid or virus, including but not limited to adenovirus (AV), adeno-associated virus (AAV), lentivirus (LV), and non-viral vectors, such as liposomes and exosome), that can be conveniently subjected to recombinant DNA procedures and can result in the expression of the engineered acid alpha-glucosidase polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. In some embodiments, the vector is a non-replicating and non-integrating vector, which may exist episomally. Furthermore, a single vector or plasmid or two or more vectors or plasmids which together contain the total DNA to be introduced into the genome of the host cell, or a transposon may be used.

In some embodiment, the recombinant polynucleotides may be provided on a non-replicating expression vector or plasmid. In some embodiments, the non-replicating expression vector or plasmid can be based on viral vectors defective in replication (see, e.g., Travieso et al., npj Vaccines, 2022, Vol. 7, Article 75).

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol, or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof. Selectable marker for mammalian cells include, but are not limited to, chloramphenicol acetyl transferase (CAT), nourseothricin N-acetyl transferase, blasticidin-S deaminase, blastcidin S acetyltransferase, Sh ble (Zeocin® resistance), aminoglycoside 3′-phosphotransferase (neomycin resistance), hph (hygromycin resistance), thymidine kinase, and puromycin N-acetyl-transferase.

In another aspect, the present disclosure provides a host cell comprising a recombinant polynucleotide encoding an engineered acid alpha-glucosidase polypeptide described herein, the polynucleotide being operably linked to one or more control sequences for expression of the engineered acid alpha-glucosidase polypeptides(s) in the host cell. In some embodiments, the host cell is a prokaryotic or eukaryotic cell. In some embodiments, the host cell is a mammalian cell. In some embodiments, the host cell is a human cell. In some embodiments, the host cell is a cell deficient in acid alpha-glucosidase activity or a cell obtained from a subject with Pompe disease.

Host cells for use in expressing the polypeptides encoded by the expression vectors of the present disclosure are known in the art and include but are not limited to, fungal cells, such as yeast cells (e.g., Saccharomyces cerevisiae and Pichia pastoris (e.g., ATCC Accession No. 201178)); insect cells (e.g., Drosophila S2 and Spodoptera Sf9 cells), plant cells, animal cells (e.g., CHO, CHO-K1, COS, and BHK), and human cells (e.g., HEK293T, human fibroblast, THP-1, Jurkat and Bowes melanoma cell lines). In some embodiments, the host cells are cells obtained from mammals, including model animals or human patients deficient in acid alpha-glucosidase activity. In some embodiments, the host cells for expression are cells obtained from human patients diagnosed with Pompe disease.

In another aspect, the present disclosure provides a method for producing the engineered acid alpha-glucosidase polypeptide, where the method comprises culturing a host cell capable of expressing a polynucleotide encoding the engineered acid alpha-glucosidase polypeptide under conditions suitable for expression of the polypeptide such that the engineered acid alpha-glucosidase polypeptide is produced. In some embodiments, the method further comprises the step of isolating the engineered acid alpha-glucosidase polypeptides, such as from media and/or cells. In some embodiments, the method further comprises purifying the expressed engineered acid alpha-glucosidase, as described herein.

Appropriate culture media and growth conditions for the above-described host cells are known in the art. Polynucleotides for expression of the engineered acid alpha-glucosidase polypeptides may be introduced into cells by various methods known in the art. Techniques include, among others, electroporation, biolistic particle bombardment, liposome mediated transfection, calcium chloride transfection, and protoplast fusion.

In some embodiments, the engineered acid alpha-glucosidase polypeptides with the properties disclosed herein can be obtained by subjecting the polynucleotide encoding the naturally occurring or engineered acid alpha-glucosidase polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 1998, 16:258-261), mutagenic PCR (See e.g., Caldwell et al., PCR Methods Appl., 1994, 3:S136-S140), and cassette mutagenesis (See e.g., Black et al., Proc. Natl. Acad. Sci. USA, 1996, 93:3525-3529).

The mutagenesis and directed evolution methods can be applied to polynucleotides to generate variant libraries that can be expressed, screened, and assayed. Any suitable mutagenesis and directed evolution methods find use in the present disclosure (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,489,146, 6,506,602, 6,506,603, 6,519,065, 6,521,453, 6,528,311, 6,537,746, 6,573,098, 6,576,467, 6,579,678, 6,586,182, 6,602,986, 6,613,514, 6,653,072, 6,716,631, 6,946,296, 6,961,664, 6,995,017, 7,024,312, 7,058,515, 7,105,297, 7,148,054, 7,288,375, 7,421,347, 7,430,477, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,873,499, 7,904,249, 7,957,912, 8,383,346, 8,504,498, 8,849,575, 8,876,066, 8,768,871, 9,593,326, and all related non-US counterparts; Ling et al., Anal. Biochem., 1997, 254(2):157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., Cell, 1984, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. U.S.A., 1997, 94:4504-4509; Crameri et al., Nat. Biotechnol., 1996, 14:315-319; Stemmer, Nature, 1994, 370:389-391; Stemmer, Proc. Nat. Acad. Sci. USA, 1994, 91:10747-10751; US Pat. Appln. Publn. Nos. 2008/0220990, US 2009/0312196, US2014/0005057, US2014/0214391, US2014/0221216; US2015/0050658, US2015/0133307, US2015/0134315 and all related non-US counterparts; WO 95/22625, WO 97/0078, WO 97/35966, WO 98/27230, WO 00/42651, WO 01/75767, and WO 2009/152336; all of which are incorporated herein by reference).

In some embodiments, the polypeptide variants obtained following mutagenesis treatment are screened by subjecting the polypeptide variants to an assay condition, such as defined temperature, acid or basic pH, serum/plasma exposure, and measuring the amount of polypeptide activity remaining after treatments or other assay conditions. DNA containing the polynucleotide encoding the engineered acid alpha-glucosidase polypeptide is then isolated from the host cell, sequenced to identify the nucleotide sequence changes (if any), and used to express the protein in a different or the same host cell. Measuring activity from the expression libraries can be performed using any suitable method known in the art and as provided in the Examples.

For engineered polypeptides of known sequence, the polynucleotides encoding the polypeptide can be prepared by standard solid-phase methods, according to known synthetic methods. In some embodiments, polynucleotide fragments can be individually synthesized, then joined (e.g., by enzymatic or chemical litigation methods, or polymerase mediated methods) to form any desired continuous sequence. For example, polynucleotides and oligonucleotides disclosed herein can be prepared by chemical synthesis using the classical phosphoramidite method (See e.g., Beaucage et al., Tetra. Lett., 1981, 22:1859-69; and Matthes et al., EMBO J., 1984, 3:801-05), as it is typically practiced in automated synthetic methods. According to the phosphoramidite method, oligonucleotides are synthesized (e.g., in an automatic DNA synthesizer), purified, annealed, ligated and cloned in appropriate vectors.

Accordingly, in some embodiments, a method for preparing the engineered acid alpha-glucosidase polypeptide can comprise: (a) synthesizing a polynucleotide encoding an engineered acid alpha-glucosidase, for example an amino acid sequence of any variant provided in Table 3-1, as well as an even-numbered SEQ ID NO. of SEQ ID NOs: 14-754, and (b) expressing the engineered acid alpha-glucosidase polypeptide encoded by the polynucleotide. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions are conservative or non-conservative substitutions.

The expressed engineered acid alpha-glucosidase polypeptide can be assessed for any desired improved property (e.g., activity/potency, stability, serum/plasma stability, basic pH tolerance, acid pH tolerance, cellular uptake, etc.), using any suitable assay known in the art, including but not limited to the assays and conditions described herein.

### Compositions

In a further aspect, the present disclosure provides compositions comprising an engineered acid alpha-glucosidase or a recombinant polynucleotide encoding the acid alpha-glucosidase, including but not limited to those compositions described below. In some embodiments, the composition comprises at least an engineered acid alpha-glucosidase or a recombinant polynucleotide exemplified in Tables 3-1 and 4-1, and the Sequence Listing.

Depending on the composition and mode of administration, compositions comprising an engineered acid alpha-glucosidase described herein are in the form of a solid, semi-solid, or liquid. In some embodiments, the compositions include other pharmaceutically acceptable components such as diluents, buffers, excipients, salts, emulsifiers, preservatives, stabilizers, fillers, and other ingredients. In some embodiments, compositions comprising the engineered acid alpha-glucosidase polypeptides of the present disclosure include one or more commonly used carrier compounds, including but not limited to sugars (e.g., lactose, sucrose, mannitol, and/or sorbitol), starches, cellulose (e.g., methyl cellulose, hydroxypropylmethyl cellulose, sodium carboxy-methylcellulose), gums (e.g., arabic, tragacanth, guar, etc.), and/or proteins (e.g., gelatin, collagen, etc.). Details on techniques for formulation and administration are available in the art and described in the literature.

In some embodiments, the engineered acid alpha-glucosidase polypeptides are formulated for use in a pharmaceutical composition. In some embodiments, the pharmaceutical composition further comprises a pharmaceutically acceptable carrier and/or excipient. Any suitable format for use in delivering an engineered acid alpha-glucosidase polypeptides find use herein, including but not limited to pills, tablets, gel tabs, capsules, lozenges, dragees, powders, soft gels, sol-gels, gels, emulsions, implants, patches, sprays, ointments, liniments, creams, pastes, jellies, paints, aerosols, chewing gums, demulcents, sticks, solutions, suspensions (including but not limited to oil-based suspensions, oil-in water emulsions, etc.), slurries, syrups, controlled release formulations, etc. In some embodiments, the engineered acid alpha-glucosidase polypeptides are provided in a format suitable for injection or infusion (i.e., in an injectable formulation), particularly for parenteral administration or infusion into a patient, particularly a human patient.

In some embodiments, the engineered acid alpha-glucosidase polypeptides are provided in biocompatible matrices such as sol-gels, including silica-based (e.g., oxysilane) sol-gels. In some embodiments, the engineered acid alpha-glucosidase polypeptides are encapsulated. In some alternative embodiments, the engineered acid alpha-glucosidase polypeptides are encapsulated in nanostructures (e.g., nanotubes, nanotubules, nanocapsules, or microcapsules, microspheres, liposomes, etc.). It is intended that the engineered acid alpha-glucosidase polypeptides be administered by any suitable means known in the art, including but not limited to parenteral (e.g., intravenous, intramuscular, subcutaneous, etc.), oral, topical, transdermal, intranasal, intraocular, intrathecal, via implants, etc.

In some embodiments, the engineered acid alpha-glucosidase polypeptides are chemically modified by glycosylation, chemical crosslinking reagents, pegylation (i.e., modified with polyethylene glycol [PEG] or activated PEG, etc.) or other compounds (See e.g., Ikeda, Amino Acids, 2005, 29:283-287; U.S. Pat. Nos. 7,531,341, 7,534,595, and 7,560,263; US Pat. Publication Nos. 2013/0039898, 2012/0177722, etc.).

In some additional embodiments, the engineered acid alpha-glucosidase polypeptides are provided in formulations comprising matrix-stabilized enzyme crystals. In some embodiments, the formulation comprises a cross-linked crystalline engineered acid alpha-glucosidase enzyme and a polymer with a reactive moiety that adheres to the enzyme crystals. The present invention also provides engineered acid alpha-glucosidase polypeptides in polymers.

In some embodiments, compositions comprising the engineered acid alpha-glucosidase polypeptides include one or more commonly used carrier compounds, including but not limited to sugars (e.g., lactose, sucrose, mannitol, and/or sorbitol), starches (e.g., corn, wheat, rice, potato, or other plant starch), cellulose (e.g., methyl cellulose, hydroxypropylmethyl cellulose, sodium carboxy-methylcellulose), gums (e.g., arabic, tragacanth, guar, etc.), and/or proteins (e.g., gelatin, collagen, etc.).

Additional components in oral formulations may include coloring and or sweetening agents (e.g., glucose, sucrose, and mannitol) and lubricating agents (e.g., magnesium stearate), as well as enteric coatings (e.g., methacrylate polymers, hydroxyl propyl methyl cellulose phthalate, and/or any other suitable enteric coating known in the art). In some embodiments, disintegrating or solubilizing agents are included (e.g., cross-linked polyvinyl pyrrolidone, agar, alginic acid or salts thereof, such as sodium alginate). In some embodiments, the engineered acid alpha-glucosidase polypeptide are be combined with various additional components, including but not limited to preservatives, suspending agents, thickening agents, wetting agents, alcohols, fatty acids, and/or emulsifiers, particularly in liquid formulations. In some embodiments, the engineered acid alpha-glucosidase polypeptides are administered to subjects in combination with other compounds, molecules, and/or materials used in the treatment of Pompe disease, including but not limited to pharmacological chaperones, as well as any other suitable compounds.

In some embodiments, the pharmaceutical composition comprises a recombinant polynucleotide encoding an engineered acid alpha-glucosidase described herein. In some embodiments, the recombinant polynucleotide is DNA or mRNA. In some embodiments, the pharmaceutical composition further comprises a pharmaceutically acceptable carrier and/or excipient suitable for administration of the recombinant polynucleotide. In some embodiments, the pharmaceutical composition comprising a recombinant polynucleotide encoding an engineered acid alpha-glucosidase is suitable for any suitable means for administration, including, among others, intravenous, intramuscular, subcutaneous, oral, intranasal, intraocular, intrathecal, inhalation, and the like.

In some additional embodiments, the recombinant polynucleotide encoding the engineered acid alpha-glucosidase polypeptides are provided for delivery to cells or tissues via gene therapy, including viral delivery vectors, including, among others, adenovirus (AV), adeno-associated virus (AAV), and lentivirus (LV). In some embodiments, the recombinant polynucleotide encoding the engineered acid alpha-glucosidase is provided for delivery in non-viral vectors or formulations, including, among others, liposomes, nanotubes, nanotubules, nanocapsules, or microcapsules, and microspheres. In some embodiments, the recombinant polynucleotide encoding the engineered acid alpha-glucosidase polypeptides are provided for delivery to cells or tissues via mRNA therapy following formulation of polyribonucleotide sequences in an encapsulated delivery, such as liposomes, or as lipid nanoparticles (see, e.g., Hou et al., Nature Reviews Materials, 2021, 6:1078-1094).

In some additional embodiments, the engineered acid alpha-glucosidase polypeptides are provided for delivery to cells or tissues via cell therapy, where the polynucleotide sequence encoding the engineered acid alpha-glucosidase polypeptides is introduced into exogenous cell and that cell (or cells) are introduced into a recipient (e.g., a patient exhibiting or at risk for developing Pompe disease). Exemplary cells that may be used include, among others, hematopoietic (blood-forming), stem cells (HSC), skeletal muscle stem cells, and mesenchymal stem cells.

### Uses and Methods

In a further aspect, the present disclosure provide use of the engineered acid alpha-glucosidase polypeptides, recombinant polynucleotides encoding the engineered acid alpha-glucosidase, or compositions thereof for treating a subject with a deficiency in acid alpha-glucosidase activity. In some embodiments, the engineered acid alpha-glucosidase polypeptides, the recombinant polynucleotides, or compositions thereof, are used for treating one or more symptoms associated with a deficiency in acid alpha-glucosidase activity. In some embodiments, the engineered acid alpha-glucosidase polypeptides, recombinant polynucleotides, or compositions thereof are used for treating a subject with Pompe disease.

In some embodiments, a method for treating and/or preventing the symptoms of a deficiency in acid alpha-glucosidase activity comprises providing to a subject in need thereof an effective amount of an engineered acid alpha-glucosidase described herein. In some embodiments, an effective amount of an engineered acid alpha-glucosidase or a pharmaceutical composition thereof is administered to the subject. In some embodiments, the engineered acid alpha-glucosidase is administered about 1 mg/kg to about 100 mg/kg. In some embodiments, the engineered acid alpha-glucosidase is administered about 10 mg/kg to about 60 mg/kg. In some embodiments, the engineered acid alpha-glucosidase is administered about 20 mg/kg to about 40 mg/kg.

In some embodiments, a recombinant polynucleotide encoding an engineered acid alpha-glucosidase, or a pharmaceutical composition thereof, is administered to a subject. In some embodiments, the recombinant polynucleotide encoding an engineered acid alpha-glucosidase, or a pharmaceutical composition thereof, is administered at a therapeutically effective amount. In some embodiments, the recombinant polynucleotide encoding the engineered acid alpha-glucosidase, or pharmaceutical compositions thereof, is administered intravenously, intramuscularly, subcutaneously, intranasally, intraocularly, intrathecally, inhalation, orally, and the like.

In some embodiments, the subject for treatment is afflicted with Pompe disease. In some embodiments, the symptoms of Pompe disease are ameliorated. In some embodiments, the subject for treatment is an infant or child. In some embodiments, the subject for treatment is an adult or young adult.

In some embodiments, the present disclosure also provides a use of the engineered acid alpha-glucosidase in the preparation of a medicament for treating a deficiency in acid alpha-glucosidase in a subject. In some embodiments, the present disclosure provides for use of a recombinant polynucleotide encoding an engineered acid alpha-glucosidase in the preparation of a medicament for treating a deficiency in acid alpha-glucosidase activity in a subject. In some embodiments, a condition involving a deficiency in acid alpha-glucosidase is Pompe disease.

The foregoing and other aspects of the invention may be better understood in connection with the following non-limiting examples. The examples are provided for illustrative purposes only and are not intended to limit the scope of the present invention in any way.

### EXAMPLES

In the experimental disclosure below, the following abbreviations apply: ppm (parts per million); M (molar); mM (millimolar), uM and μM (micromolar); nM (nanomolar); mol (moles); gm and g (gram); mg (milligrams); ug and μg (micrograms); L and 1 (liter); ml and mL (milliliter); ul, μl, uL, μL (microliter); cm (centimeters); mm (millimeters); um and μm (micrometers); sec. (seconds); min(s) (minute(s)); h(s) and hr(s) (hour(s)); U (units); MW (molecular weight); rpm (rotations per minute); ° C. (degrees Celsius); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); DPBS (Dulbecco's phosphate buffered saline); LB (Luria-Burtani); TB (terrific broth); 4-MUGlu or 4-MU-GLU (4-methylumbelliferyl α-D-glucopyranoside; SD-Ura (single drop out medium without uracil); HPLC (high pressure liquid chromatography); SDS-PAGE (sodium dodecyl sulfate polyacrylamide gel electrophoresis); MU-Glu (4-methylumbelliferyl α-D-glucopyranoside); IPTG (isopropyl β-D-1-thiogalactopyranoside); PMBS (polymyxin B sulfate); FIOPC (fold improvements over positive control); PBMC (peripheral blood mononuclear cells); LB (Luria broth); and MeOH (methanol).

### Example 1

### GAA Gene Acquisition and Construction of Expression Vectors

This example describes GAA gene acquisition and expression vector construction. A synthetic gene coding for a WT human GAA (Uniprot ID P10253) was designed for optimized gene expression in Homo sapiens (SEQ ID NO: 1) and cloned into the expression vector pDH (see International patent publication WO2021127457). For secreted expression and transient transfection in mammalian cells, a chimeric GAA expression construct encoding the synthetic mouse IG signal peptide (residues 1-19 of Uniprot accession number: AON1R5) fused to a synthetic gene coding for the different GAA variants was generated as follows. Oligonucleotides containing restriction enzyme flanks to enable cloning into either the BamHI/XhoI site or HindIII/XhoI site were used to amplify a fragment coding for the synthetic mouse IG signal peptide and the coding sequence for the mature form of GAA. Acid alpha-glucosidase variants SEQ ID NO: 3, 7, 5, 13, 9 were cloned into pDH or pcDNA3.1(+). Directed evolution was used to generate specific gene variants derived from SEQ ID NO: 11 (disclosed in WO2021127457 as SEQ ID NO: 3103 for polynucleotide sequence and SEQ ID NO: 3104 for polypeptide sequence) within the pDH plasmid construct (see e.g., U.S. Pat. No. 8,383,346 and WO2010/144103).

### Example 2

### High-Throughput Growth of Suspension Mammalian Cells and GAA Assays Obtained Through Suspension Mammalian Expression

### High-Throughput (HTP) Growth of GAA and GAA Variants in Suspension Mammalian Cells (Expi293F)

EXPI293F™ cells (ThermoFisher Scientific) were transfected with polynucleotides encoding wild-type GAA or GAA variants having a synthetic mouse IG signal peptide fusion using the lipofection method with EXPIFECTAMINE™ 293 Reagent (ThermoFisher Scientific) in EXPI293™ Expression Medium (ThermoFisher Scientific). EXPI293F™ cells (ThermoFisher Scientific) were cultured in EXPI293™ Expression Medium (ThermoFisher Scientific) and seeded into Axygen 1.1 mL deep well plate (Corning, P-DW-11-C-S), at densities of 1×10⁶ cells/well/400 μL. Cells were subjected to lipofection-mediated transfection and returned to a shaking incubator with 8% CO₂ and 70% humidity for 3-4 days to allow for expression and secretion of GAA variants into the conditioned media. Conditioned media was harvested by centrifugation of expression plates and transfer of conditioned media into a BioRad Hardshell PCR Plate (BioRad, HSP9601). Plates were centrifuged again and clarified conditioned media was transferred into new 96-well plates for activity, stability or uptake into cell analysis.

### HTP-Analysis of Supernatants

GAA variant activity was determined by measuring the hydrolysis of 4-methylumbelliferyl α-D-glucopyranoside (4-MU-GLU). For the unchallenged assay, 5 μL of EXPI293F™ clarified conditioned media produced as described above was mixed with 50 μL of 1.5 mM 4-MU-GLU in McIlvaine Buffer (McIlvaine, J. Biol. Chem., 1921, 49:183-186), pH 4.4, in a 96-well, black, opaque bottom plate. The reactions were incubated at 25° C. for 15 minutes with agitation at 400 rpm, prior to quenching with 100 μL of 0.5M sodium carbonate pH 10.5. Hydrolysis was analyzed using an EnVision microplate reader (Perkin Elmer) monitoring fluorescence (Ex. 355 nm, Em. 460 nm). Unchallenged activity FIOPC was calculated by dividing normalized GAA variant by the activity of the reference polypeptide with the indicated SEQ ID NO.

HTP-Analysis of Supernatants Challenged with Plasma

GAA variants were challenged with plasma to simulate the conditions that the variants encounter in the blood following their administration to a patient. First, 30 μL of GAA variants in EXPI293F™ clarified conditioned media were combined with 30 μL of plasma (Innovative Research, Innovative Grade US Origin Monkey Cynomolgus Plasma K2 EDTA) in a 96-well plate. The plates were sealed and incubated at 37° C. with agitation at 400 rpm for 4 hours. Next, 10 μL of plasma-challenged sample were mixed with 50 μL of 1.5 mM 4-MU-GLU in McIlvaine buffer, pH 4.4. The reactions were incubated at 25-37° C. for 30 minutes with agitation at 400 rpm, prior to quenching with 100 μL of 0.5M sodium carbonate pH 10.5. Hydrolysis was analyzed using an EnVision microplate reader (Perkin Elmer) monitoring fluorescence (Ex. 355 nm, Em. 460 nm). Plasma stability FIOPC was calculated by dividing normalized GAA variant activity following challenge by the activity of the reference polypeptide with the indicated SEQ ID NO following challenge.

### HTP-Analysis of GAA Activity in Lysates of Pompe Fibroblasts

GAA variants from HTP EXPI293F™ expression in clarified conditioned media were incubated with target cells and assayed for residual intracellular activity after 72 hours. For these experiments, mammalian cells lacking functional GAA activity were used, namely Pompe patient-derived fibroblasts (Coriell Institute for Medical Research, #GM00248). Pompe patient-derived fibroblasts were seeded into 96-well plates COSTAR® (3904, Corning) and allowed to grow to confluency in standard complete growth medium. Upon confluency, complete growth culture media was removed from the plates using an automated BioMek i5 liquid handler. Clarified conditioned media from transient HPT transfections in EXPI293F™, were transferred to Pompe patient-derived fibroblasts, and allowed to incubate for 24 hours at 37° C., 5% CO₂. Medium was removed from the cultures using an automated BioMek i5 liquid handler. The cells were briefly washed with 150 μL 1×DPBS/well, and DPBS was removed using an automated BioMek i5 liquid handler. Then, 200 μL standard complete growth culture medium was added to each well, and the plates were returned to the incubator for 72 hours. At the conclusion of incubation, standard complete growth media was removed using an automated BioMek i5 liquid handler. The cells were washed with 150 μL 1×DPBS/well, and the DPBS removed using an automated BioMek i5 liquid handling. The cells were lysed via addition of 50 μL of McIlvaine buffer, pH 4.4, supplemented with 0.5% TRITON X-100^{T}M non-ionic surfactant (Sigma, Cat #93443) and agitation at room temperature for 30 minutes. Activity was assessed by addition of 50 μL of 1.5 mM 4-MU-GLU in McIlvaine buffer, pH 4.4. The plates were sealed, incubated at 37° C. for 360 minutes with agitation at 400 rpm, prior to quenching with 100 μL of 0.5M sodium carbonate, pH 10.5. Hydrolysis was analyzed using an EnVision microplate reader (Perkin Elmer) monitoring fluorescence (Ex. 355 nm, Em. 460 nm). Cellular uptake FIOPC was calculated by dividing normalized GAA variant intracellular activity by the activity of the reference polypeptide with the indicated SEQ ID NO.

### Example 3

### GAA Variants of SEQ ID NO: 11

In this Example, experiments for evolution and screening of GAA variants derived from SEQ ID NO: 11 for improved GAA activity after a series of challenges are described. Libraries of variant genes GAA encoded based on SEQ ID NO: 11 were constructed, plated, grown, and screened for GAA 4-MU-GLU activity (“Unchallenged Activity FOPC”), as well as after plasma challenge (“Plasma Stability and Activity FOPC”), as described in Example 2. Variants were also tested for 4-MU-GLU activity after lysis of treatments of Pompe fibroblasts with conditioned media (“Lysate Activity from Treatment of Pompe Fibroblast FIOPC”), as described in Example 2. The results of these assays are presented in Table 3-1.

TABLE 3-1
Activity of GAA Variants Under Various Conditions Relative to SEQ ID NO: 12¹

| | | | | | Lysate |
|---|---|---|---|---|---|
| | | | | | Activity from |
| | Amino Acid | Amino Acid | | Plasma | Treatment |
| SEQ ID | Differences | Differences | Unchallenged | Stability | of Pompe |
| NO: | (Relative to | Relative to | Activity | and Activity | Fibroblast |
| (nt/aa) | SEQ ID NO: 2) | SEQ ID NO: 12 | FIOPC | FIOPC | FIOPC |
| 13/14 | L24W/L28S/L29T/P39Q/Q50V/ | L305V | + | + | + |
| | A62L/P78E/D87E/S135Q/T150S/ | | | | |
| | T266N/R267K/L305V/A437G/T486E/ | | | | |
| | E522V/L569T/L670T/T692G/ | | | | |
| | A711H/L736M/A750P/A812E/ | | | | |
| | Q830K/G842S/L871E/R883H/ | | | | |
| | Q894G/V913R/S932A | | | | |
| 15/16 | L24W/L28S/L29T/P39Q/Q50V/ | Q135P | | + | |
| | A62L/P78E/D87E/S135P/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 17/18 | L24W/L28S/L29T/P39Q/Q50V/ | E486Q | + | + | + |
| | A62L/P78E/D87E/S135Q/T150S/ | | | | |
| | T266N/R267K/A437G/T486Q/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 19/20 | L24W/L28S/L29T/P39Q/Q50V/ | S842E | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842E/L871E/R883H/Q894G/V913R/ | | | | |
| | S932A | | | | |
| 21/22 | L24W/L28S/L29T/P39Q/Q50V/ | E78G | + | + | + |
| | A62L/P78G/D87E/S135Q/T150S/ | | | | |
| | T266N/R267K/A437G/T486E/ | | | | |
| | E522V/L569T/L670T/T692G/ | | | | |
| | A711H/L736M/A750P/A812E/Q830K/ | | | | |
| | G842S/L871E/R883H/Q894G/ | | | | |
| | V913R/S932A | | | | |
| 23/24 | L24W/L28S/L29T/P39Q/Q50V/ | G894S | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894S/ | | | | |
| | V913R/S932A | | | | |
| 25/26 | L24W/L28S/L29A/P39Q/Q50V/ | T29A | + | + | |
| | A62L/P78E/D87E/S135Q/T150S/ | | | | |
| | T266N/R267K/A437G/T486E/ | | | | |
| | E522V/L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 27/28 | L24W/L28S/L29T/P39Q/Q50V/ | S842G | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | L871E/R883H/Q894G/V913R/S932A | | | | |
| 29/30 | L24W/L28S/L29T/P39Q/Q50V/ | L62S | | + | |
| | A62S/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 31/32 | L24W/L28S/L29T/P39Q/Q50V/ | A932R | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932R | | | | |
| 33/34 | L24W/L28A/L29T/P39Q/Q50V/ | S28A | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 35/36 | L24W/L28S/L29T/P39Q/Q50V/ | T569A | + | + | + |
| | A62L/P78E/D87E/S135Q/T150S/ | | | | |
| | T266N/R267K/A437G/T486E/E522V/ | | | | |
| | L569A/L670T/T692G/A711H/ | | | | |
| | L736M/A750P/A812E/Q830K/ | | | | |
| | G842S/L871E/R883H/Q894G/V913R/ | | | | |
| | S932A | | | | |
| 37/38 | L24W/L28S/L29T/P39Q/Q50V/ | T569R | + | | |
| | A62L/P78E/D87E/S135Q/T150S/ | | | | |
| | T266N/R267K/A437G/T486E/E522V/ | | | | |
| | L569R/L670T/T692G/A711H/ | | | | |
| | L736M/A750P/A812E/Q830K/ | | | | |
| | G842S/L871E/R883H/Q894G/V913R/ | | | | |
| | S932A | | | | |
| 39/40 | L24W/L28S/L29T/P39Q/Q50V/ | S842W | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842W/L871E/R883H/Q894G/V913R/ | | | | |
| | S932A | | | | |
| 41/42 | L24W/L28S/L29C/P39Q/Q50V/ | T29C | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 43/44 | L24W/L28S/L29T/P39Q/Q50V/ | K267L | | | + |
| | A62L/P78E/D87E/S135Q/T150S/ | | | | |
| | T266N/R267L/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 45/46 | L24W/L28S/L29T/P39Q/Q50V/ | E87Q | | + | ++ |
| | A62L/P78E/D87Q/S135Q/T150S/ | | | | |
| | T266N/R267K/A437G/T486E/ | | | | |
| | E522V/L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 47/48 | L24W/L28S/L29T/P39Q/Q50V/ | L62D | + | + | |
| | A62D/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 49/50 | L24W/L28S/L29T/P39Q/Q50V/ | L62H | + | ++ | |
| | A62H/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 51/52 | L24W/L28S/L29T/P39Q/Q50V/ | Q135R | + | + | ++ |
| | A62L/P78E/D87E/S135R/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 53/54 | L28S/L29T/P39Q/Q50V/A62L/P78E/ | W24L | ++ | + | + |
| | D87E/S135Q/T150S/T266N/ | | | | |
| | R267K/A437G/T486E/E522V/L569T/ | | | | |
| | L670T/T692G/A711H/L736M/ | | | | |
| | A750P/A812E/Q830K/G842S/ | | | | |
| | L871E/R883H/Q894G/V913R/S932A | | | | |
| 55/56 | L24W/L28S/L29T/P39Q/Q50V/ | E871A | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871A/R883H/Q894G/ | | | | |
| | V913R/S932A | | | | |
| 57/58 | L24W/L28S/L29T/P39Q/Q50S/A62L/ | V50S | + | + | |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 59/60 | L24W/L28S/L29T/P39Q/Q50V/P78E/ | L62A | + | + | |
| | D87E/S135Q/T150S/T266N/ | | | | |
| | R267K/A437G/T486E/E522V/L569T/ | | | | |
| | L670T/T692G/A711H/L736M/ | | | | |
| | A750P/A812E/Q830K/G842S/ | | | | |
| | L871E/R883H/Q894G/V913R/S932A | | | | |
| 61/62 | L24W/L28S/L29T/P39Q/Q50V/ | T569G | | + | |
| | A62L/P78E/D87E/S135Q/T150S/ | | | | |
| | T266N/R267K/A437G/T486E/E522V/ | | | | |
| | L569G/L670T/T692G/A711H/ | | | | |
| | L736M/A750P/A812E/Q830K/ | | | | |
| | G842S/L871E/R883H/Q894G/V913R/ | | | | |
| | S932A | | | | |
| 63/64 | L24W/L28S/L29T/P39Q/Q50V/ | G692R | + | + | + |
| | A62L/P78E/D87E/S135Q/T150S/ | | | | |
| | T266N/R267K/A437G/T486E/E522V/ | | | | |
| | L569T/L670T/T692R/A711H/ | | | | |
| | L736M/A750P/A812E/Q830K/ | | | | |
| | G842S/L871E/R883H/Q894G/V913R/ | | | | |
| | S932A | | | | |
| 65/66 | L24W/L28S/L29T/P39Q/Q50V/ | K830N | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830N/ | | | | |
| | G842S/L871E/R883H/Q894G/V913R/ | | | | |
| | S932A | | | | |
| 67/68 | L24W/L28S/L29T/P39Q/Q50V/ | G894R | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894R/V913R/ | | | | |
| | S932A | | | | |
| 69/70 | L24W/L28S/L29T/P39Q/Q50V/ | K267T | | | ++ |
| | A62L/P78E/D87E/S135Q/T150S/ | | | | |
| | T266N/R267T/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 71/72 | L24E/L28S/L29T/P39Q/Q50V/A62L/ | W24E | +++ | +++ | ++ |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/G842S/ | | | | |
| | L871E/R883H/Q894G/V913R/ | | | | |
| | S932A | | | | |
| 73/74 | L24W/L28S/L29T/P39Q/Q50V/ | Q135G | | + | |
| | A62L/P78E/D87E/S135G/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 75/76 | L24W/L28S/L29T/P39Q/Q50V/ | G692V | + | + | |
| | A62L/P78E/D87E/S135Q/T150S/ | | | | |
| | T266N/R267K/A437G/T486E/E522V/ | | | | |
| | L569T/L670T/T692V/A711H/ | | | | |
| | L736M/A750P/A812E/Q830K/ | | | | |
| | G842S/L871E/R883H/Q894G/V913R/ | | | | |
| | S932A | | | | |
| 77/78 | L24W/L28S/L29T/P39Q/Q50V/ | E87R | | | + |
| | A62L/P78E/D87R/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 79/80 | L24W/L28S/L29T/P39Q/Q50V/ | R913Q | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913Q/ | | | | |
| | S932A | | | | |
| 81/82 | L24W/L28S/L29T/P39Q/Q50V/ | H711L | + | ++ | + |
| | A62L/P78E/D87E/S135Q/T150S/ | | | | |
| | T266N/R267K/A437G/T486E/E522V/ | | | | |
| | L569T/L670T/T692G/A711L/ | | | | |
| | L736M/A750P/A812E/Q830K/ | | | | |
| | G842S/L871E/R883H/Q894G/V913R/ | | | | |
| | S932A | | | | |
| 83/84 | L24W/L28S/L29T/P39Q/Q50V/ | E87L | | | + |
| | A62L/P78E/D87L/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R/ | | | | |
| | S932A | | | | |
| 85/86 | L24W/L28S/L29T/P39Q/Q50V/ | E87V | | + | + |
| | A62L/P78E/D87V/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 87/88 | L24W/L28S/L29T/P39Q/Q50V/ | E78Y | | + | +++ |
| | A62L/P78Y/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 89/90 | L24W/L28S/L29T/P39Q/Q50V/ | T569C | + | + | |
| | A62L/P78E/D87E/S135Q/T150S/ | | | | |
| | T266N/R267K/A437G/T486E/E522V/ | | | | |
| | L569C/L670T/T692G/A711H/ | | | | |
| | L736M/A750P/A812E/Q830K/ | | | | |
| | G842S/L871E/R883H/Q894G/V913R/ | | | | |
| | S932A | | | | |
| 91/92 | L24W/L28S/L29T/P39Q/Q50V/ | E87W | | | + |
| | A62L/P78E/D87W/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 93/94 | L24W/L28S/L29T/P39Q/Q50V/ | T670A | ++ | + | + |
| | A62L/P78E/D87E/S135Q/T150S/ | | | | |
| | T266N/R267K/A437G/T486E/E522V/ | | | | |
| | L569T/L670A/T692G/A711H/ | | | | |
| | L736M/A750P/A812E/Q830K/ | | | | |
| | G842S/L871E/R883H/Q894G/ | | | | |
| | V913R/S932A | | | | |
| 95/96 | L24W/L28S/L29T/P39Q/Q50V/ | T569Q | ++ | + | |
| | A62L/P78E/D87E/S135Q/T150S/ | | | | |
| | T266N/R267K/A437G/T486E/E522V/ | | | | |
| | L569Q/L670T/T692G/A711H/ | | | | |
| | L736M/A750P/A812E/Q830K/ | | | | |
| | G842S/L871E/R883H/Q894G/ | | | | |
| | V913R/S932A | | | | |
| 97/98 | L24W/L28S/L29T/P39Q/Q50V/ | L62K | + | + | + |
| | A62K/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 99/100 | L24W/L28S/L29T/P39Q/Q50V/ | G894C | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894C/ | | | | |
| | V913R/S932A | | | | |
| 101/102 | L24W/L28S/L29T/P39Q/Q50V/ | T569H | + | + | |
| | A62L/P78E/D87E/S135Q/T150S/ | | | | |
| | T266N/R267K/A437G/T486E/E522V/ | | | | |
| | L569H/L670T/T692G/A711H/ | | | | |
| | L736M/A750P/A812E/Q830K/ | | | | |
| | G842S/L871E/R883H/Q894G/ | | | | |
| | V913R/S932A | | | | |
| 103/104 | L24W/L28S/L29T/P39Q/Q50V/ | H711V | + | + | |
| | A62L/P78E/D87E/S135Q/T150S/ | | | | |
| | T266N/R267K/A437G/T486E/E522V/ | | | | |
| | L569T/L670T/T692G/A711V/ | | | | |
| | L736M/A750P/A812E/Q830K/ | | | | |
| | G842S/L871E/R883H/Q894G/ | | | | |
| | V913R/S932A | | | | |
| 105/106 | L24W/L28S/L29T/P39Q/Q50V/ | V522S | + | | |
| | A62L/P78E/D87E/S135Q/T150S/ | | | | |
| | T266N/R267K/A437G/T486E/E522S/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 107/108 | L24F/L28S/L29T/P39Q/Q50V/ | W24F | ++ | ++ | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 109/110 | L24W/L28S/L29T/P39Q/Q50V/ | T569M | ++ | + | ++ |
| | A62L/P78E/D87E/S135Q/T150S/ | | | | |
| | T266N/R267K/A437G/T486E/E522V/ | | | | |
| | L569M/L670T/T692G/A711H/ | | | | |
| | L736M/A750P/A812E/Q830K/ | | | | |
| | G842S/L871E/R883H/Q894G/ | | | | |
| | V913R/S932A | | | | |
| 111/112 | L24W/L28S/L29T/P39Q/Q50V/ | H711S | ++ | ++ | + |
| | A62L/P78E/D87E/S135Q/T150S/ | | | | |
| | T266N/R267K/A437G/T486E/ | | | | |
| | E522V/L569T/L670T/T692G/A711S/ | | | | |
| | L736M/A750P/A812E/Q830K/ | | | | |
| | G842S/L871E/R883H/Q894G/ | | | | |
| | V913R/S932A | | | | |
| 113/114 | L24Y/L28S/L29T/P39Q/Q50V/ | W24Y | +++ | +++ | +++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 115/116 | L24W/L28S/L29T/P39Q/Q50V/ | E87G | + | + | ++ |
| | A62L/P78E/D87G/S135Q/T150S/ | | | | |
| | T266N/R267K/A437G/T486E/ | | | | |
| | E522V/L569T/L670T/T692G/ | | | | |
| | A711H/L736M/A750P/A812E/Q830K/ | | | | |
| | G842S/L871E/R883H/Q894G/ | | | | |
| | V913R/S932A | | | | |
| 117/118 | L24W/L28S/L29T/P39Q/Q50V/ | E78I | + | + | + |
| | A62L/P78I/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 119/120 | L24W/L28S/L29T/P39Q/Q50V/ | E871L | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/R883H/Q894G/V913R/S932A | | | | |
| 121/122 | L24W/L28S/L29T/P39Q/Q50V/ | E78R | + | + | ++ |
| | A62L/P78R/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 123/124 | L24W/L28S/L29T/P39Q/Q50V/ | T569L | + | + | |
| | A62L/P78E/D87E/S135Q/T150S/ | | | | |
| | T266N/R267K/A437G/T486E/E522V/ | | | | |
| | L670T/T692G/A711H/L736M/ | | | | |
| | A750P/A812E/Q830K/G842S/ | | | | |
| | L871E/R883H/Q894G/V913R/S932A | | | | |
| 125/126 | L24C/L28S/L29T/P39Q/Q50V/A62L/ | W24C | +++ | +++ | +++ |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 127/128 | L24W/L28S/L29T/P39Q/Q50V/ | T670L | + | + | + |
| | A62L/P78E/D87E/S135Q/T150S/ | | | | |
| | T266N/R267K/A437G/T486E/E522V/ | | | | |
| | L569T/T692G/A711H/L736M/ | | | | |
| | A750P/A812E/Q830K/G842S/ | | | | |
| | L871E/R883H/Q894G/V913R/S932A | | | | |
| 129/130 | L24W/L28S/L29T/P39Q/Q50V/ | E78W | + | + | +++ |
| | A62L/P78W/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 131/132 | L24W/L28S/L29T/P39Q/Q50V/ | V522F | + | | |
| | A62L/P78E/D87E/S135Q/T150S/ | | | | |
| | T266N/R267K/A437G/T486E/E522F/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 133/134 | L24W/L28S/L29T/P39Q/Q50V/ | V522P | + | | |
| | A62L/P78E/D87E/S135Q/T150S/ | | | | |
| | T266N/R267K/A437G/T486E/E522P/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 135/136 | L24W/L28S/L29T/P39Q/Q50V/ | K830V | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830V/ | | | | |
| | G842S/L871E/R883H/Q894G/ | | | | |
| | V913R/S932A | | | | |
| 137/138 | L24W/L28S/L29T/P39Q/Q50V/ | H711M | + | + | + |
| | A62L/P78E/D87E/S135Q/T150S/ | | | | |
| | T266N/R267K/A437G/T486E/E522V/ | | | | |
| | L569T/L670T/T692G/A711M/ | | | | |
| | L736M/A750P/A812E/Q830K/ | | | | |
| | G842S/L871E/R883H/Q894G/ | | | | |
| | V913R/S932A | | | | |
| 139/140 | L24W/L28T/L29T/P39Q/Q50V/ | S28T | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 141/142 | L24W/L28S/L29T/P39Q/Q50V/ | T569K | ++ | + | + |
| | A62L/P78E/D87E/S135Q/T150S/ | | | | |
| | T266N/R267K/A437G/T486E/E522V/ | | | | |
| | L569K/L670T/T692G/A711H/ | | | | |
| | L736M/A750P/A812E/Q830K/ | | | | |
| | G842S/L871E/R883H/Q894G/ | | | | |
| | V913R/S932A | | | | |
| 143/144 | L24S/L28S/L29T/P39Q/Q50V/ | W24S | ++ | ++ | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 145/146 | L24W/L28S/L29T/P39Q/Q50V/ | Q135L | + | + | + |
| | A62L/P78E/D87E/S135L/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 147/148 | L24W/L28S/L29T/P39Q/Q50V/ | S842R | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842R/L871E/R883H/Q894G/ | | | | |
| | V913R/S932A | | | | |
| 149/150 | L24W/L28S/L29T/P39Q/Q50V/ | G894Y | + | ++ | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894Y/ | | | | |
| | V913R/S932A | | | | |
| 151/152 | L24W/L28S/L29T/P39Q/Q50V/ | S842T | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842T/L871E/R883H/Q894G/ | | | | |
| | V913R/S932A | | | | |
| 153/154 | L24W/L28S/L29T/P39Q/Q50V/ | G894T | + | ++ | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894T/ | | | | |
| | V913R/S932A | | | | |
| 155/156 | L24W/L28Q/L29T/P39Q/Q50V/ | S28Q | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 157/158 | L24W/L28S/L29T/P39Q/Q50V/ | H711A | ++ | ++ | ++ |
| | A62L/P78E/D87E/S135Q/T150S/ | | | | |
| | T266N/R267K/A437G/T486E/E522V/ | | | | |
| | L569T/L670T/T692G/L736M/ | | | | |
| | A750P/A812E/Q830K/G842S/ | | | | |
| | L871E/R883H/Q894G/V913R/S932A | | | | |
| 159/160 | L24W/L28S/L29T/P39Q/Q50V/ | N266H | + | | + |
| | A62L/P78E/D87E/S135Q/T150S/ | | | | |
| | T266H/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 161/162 | L24W/L28S/L29T/P39Q/Q50V/ | L62P | + | + | |
| | A62P/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 163/164 | L24W/L28S/L29T/P39L/Q50V/ | Q39L | + | + | +++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 165/166 | L24W/L28S/L29T/P39Q/Q50V/ | E486D | + | + | ++ |
| | A62L/P78E/D87E/S135Q/T150S/ | | | | |
| | T266N/R267K/A437G/T486D/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 167/168 | L24W/L28S/L29T/P39Q/Q50V/ | H711G | + | + | + |
| | A62L/P78E/D87E/S135Q/T150S/ | | | | |
| | T266N/R267K/A437G/T486E/E522V/ | | | | |
| | L569T/L670T/T692G/A711G/ | | | | |
| | L736M/A750P/A812E/Q830K/ | | | | |
| | G842S/L871E/R883H/Q894G/ | | | | |
| | V913R/S932A | | | | |
| 169/170 | L24N/L28S/L29T/P39Q/Q50V/ | W24N | +++ | +++ | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 171/172 | L24W/L28S/L29T/P39Q/Q50V/ | H711W | + | + | |
| | A62L/P78E/D87E/S135Q/T150S/ | | | | |
| | T266N/R267K/A437G/T486E/E522V/ | | | | |
| | L569T/L670T/T692G/A711W/ | | | | |
| | L736M/A750P/A812E/Q830K/ | | | | |
| | G842S/L871E/R883H/Q894G/ | | | | |
| | V913R/S932A | | | | |
| 173/174 | L24W/L28S/L29T/P39Q/Q50V/ | E871R | + | ++ | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871R/R883H/Q894G/ | | | | |
| | V913R/S932A | | | | |
| 175/176 | L24W/L28S/L29T/P39Q/Q50V/ | M736L | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | A750P/A812E/Q830K/G842S/ | | | | |
| | L871E/R883H/Q894G/V913R/S932A | | | | |
| 177/178 | L24W/L28S/L29T/P39Q/Q50V/ | V522W | + | | + |
| | A62L/P78E/D87E/S135Q/T150S/ | | | | |
| | T266N/R267K/A437G/T486E/E522W/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 179/180 | L24W/L28S/L29T/P39Q/Q50V/ | G894L | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894L/ | | | | |
| | V913R/S932A | | | | |
| 181/182 | L24W/L28S/L29T/P39Q/Q50V/ | H883A | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883A/Q894G/ | | | | |
| | V913R/S932A | | | | |
| 183/184 | L24W/L28S/L29T/P39Q/Q50V/ | V522G | + | | |
| | A62L/P78E/D87E/S135Q/T150S/ | | | | |
| | T266N/R267K/A437G/T486E/E522G/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 185/186 | L24W/L28S/L29T/P39Q/Q50V/ | V522L | | + | |
| | A62L/P78E/D87E/S135Q/T150S/ | | | | |
| | T266N/R267K/A437G/T486E/E522L/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 187/188 | L24W/L28S/L29T/P39Q/Q50V/ | T670V | ++ | ++ | + |
| | A62L/P78E/D87E/S135Q/T150S/ | | | | |
| | T266N/R267K/A437G/T486E/E522V/ | | | | |
| | L569T/L670V/T692G/A711H/ | | | | |
| | L736M/A750P/A812E/Q830K/ | | | | |
| | G842S/L871E/R883H/Q894G/ | | | | |
| | V913R/S932A | | | | |
| 189/190 | L24W/L28S/L29T/P39Q/Q50V/ | L62G | | + | |
| | A62G/P78E/D87E/S135Q/T150S/ | | | | |
| | T266N/R267K/A437G/T486E/ | | | | |
| | E522V/L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 191/192 | L24W/L28S/L29T/P39Q/Q50V/ | G692N | + | + | |
| | A62L/P78E/D87E/S135Q/T150S/ | | | | |
| | T266N/R267K/A437G/T486E/E522V/ | | | | |
| | L569T/L670T/T692N/A711H/ | | | | |
| | L736M/A750P/A812E/Q830K/ | | | | |
| | G842S/L871E/R883H/Q894G/ | | | | |
| | V913R/S932A | | | | |
| 193/194 | L24W/L28S/L29T/P39Q/Q50V/ | T569V | ++ | + | ++ |
| | A62L/P78E/D87E/S135Q/T150S/ | | | | |
| | T266N/R267K/A437G/T486E/E522V/ | | | | |
| | L569V/L670T/T692G/A711H/ | | | | |
| | L736M/A750P/A812E/Q830K/ | | | | |
| | G842S/L871E/R883H/Q894G/ | | | | |
| | V913R/S932A | | | | |
| 195/196 | L24W/L28S/L29T/P39Q/Q50V/ | T670I | + | + | |
| | A62L/P78E/D87E/S135Q/T150S/ | | | | |
| | T266N/R267K/A437G/T486E/E522V/ | | | | |
| | L569T/L670I/T692G/A711H/ | | | | |
| | L736M/A750P/A812E/Q830K/ | | | | |
| | G842S/L871E/R883H/Q894G/ | | | | |
| | V913R/S932A | | | | |
| 197/198 | L24W/L28S/L29T/P39Q/Q50V/ | E87A | + | + | + |
| | A62L/P78E/D87A/S135Q/T150S/ | | | | |
| | T266N/R267K/A437G/T486E/ | | | | |
| | E522V/L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 199/200 | L24W/L28S/L29T/P39Q/Q50V/ | G894A | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894A/ | | | | |
| | V913R/S932A | | | | |
| 201/202 | L24W/L28S/L29T/P39Q/Q50V/ | N266A | + | | |
| | A62L/P78E/D87E/S135Q/T150S/ | | | | |
| | T266A/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 203/204 | L24W/L28S/L29T/P39Q/Q50V/ | V522E | + | + | |
| | A62L/P78E/D87E/S135Q/T150S/ | | | | |
| | T266N/R267K/A437G/T486E/L569T/ | | | | |
| | L670T/T692G/A711H/L736M/ | | | | |
| | A750P/A812E/Q830K/G842S/ | | | | |
| | L871E/R883H/Q894G/V913R/S932A | | | | |
| 205/206 | L24W/L28R/L29T/P39Q/Q50V/ | S28R | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 207/208 | L24W/L28S/L29T/P39Q/Q50V/ | E871G | | | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871G/R883H/Q894G/ | | | | |
| | V913R/S932A | | | | |
| 209/210 | L24W/L28S/L29T/P39Q/Q50V/ | V522I | + | ++ | |
| | A62L/P78E/D87E/S135Q/T150S/ | | | | |
| | T266N/R267K/A437G/T486E/E522I/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 211/212 | L24W/L28S/L29T/P39Q/Q50V/ | H711Y | + | + | + |
| | A62L/P78E/D87E/S135Q/T150S/ | | | | |
| | T266N/R267K/A437G/T486E/ | | | | |
| | E522V/L569T/L670T/T692G/A711Y/ | | | | |
| | L736M/A750P/A812E/Q830K/ | | | | |
| | G842S/L871E/R883H/Q894G/ | | | | |
| | V913R/S932A | | | | |
| 213/214 | L24W/L28S/L29T/P39Q/Q50E/A62L/ | V50E | ++ | ++ | |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 215/216 | L24W/L28S/L29T/P39Q/Q50V/ | L62V | + | + | |
| | A62V/P78E/D87E/S135Q/T150S/ | | | | |
| | T266N/R267K/A437G/T486E/ | | | | |
| | E522V/L569T/L670T/T692G/ | | | | |
| | A711H/L736M/A750P/A812E/Q830K/ | | | | |
| | G842S/L871E/R883H/Q894G/ | | | | |
| | V913R/S932A | | | | |
| 217/218 | L24W/L28S/L29T/P39Q/Q50V/ | E87S | | | + |
| | A62L/P78E/D87S/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 219/220 | L24W/L28S/L29T/P39Q/Q50V/ | L62N | + | + | |
| | A62N/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 221/222 | L24W/L28S/L29T/P39Q/Q50V/ | E87H | | | ++ |
| | A62L/P78E/D87H/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 223/224 | L24W/L28S/L29T/P39Q/Q50V/ | E486L | + | + | ++ |
| | A62L/P78E/D87E/S135Q/T150S/ | | | | |
| | T266N/R267K/A437G/T486L/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 225/226 | L24W/L28S/L29T/P39Q/Q50V/ | R913E | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913E/S932A | | | | |
| 227/228 | L24W/L28S/L29T/P39Q/Q50V/ | H711R | ++ | ++ | + |
| | A62L/P78E/D87E/S135Q/T150S/ | | | | |
| | T266N/R267K/A437G/T486E/E522V/ | | | | |
| | L569T/L670T/T692G/A711R/ | | | | |
| | L736M/A750P/A812E/Q830K/ | | | | |
| | G842S/L871E/R883H/Q894G/ | | | | |
| | V913R/S932A | | | | |
| 229/230 | L24W/L28S/L29T/P39G/Q50V/ | Q39G | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 231/232 | L24R/L28S/L29T/P39Q/Q50V/A62L/ | W24R | +++ | ++ | + |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 233/234 | L24W/L28S/L29T/P39Q/Q50V/ | N266D | | + | |
| | A62L/P78E/D87E/S135Q/T150S/ | | | | |
| | T266D/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 235/236 | L24W/L28S/L29T/P39T/Q50V/ | Q39T | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 237/238 | L24W/L28S/L29T/P39Q/Q50C/ | V50C | ++ | ++ | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 239/240 | L24A/L28S/L29T/P39Q/Q50V/ | W24A | +++ | +++ | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 241/242 | L24W/L28S/L29T/P39Q/Q50V/ | E87K | | | + |
| | A62L/P78E/D87K/S135Q/T150S/ | | | | |
| | T266N/R267K/A437G/T486E/ | | | | |
| | E522V/L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 243/244 | L24W/L28S/L29T/P39Q/Q50V/ | P750A | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A812E/Q830K/G842S/ | | | | |
| | L871E/R883H/Q894G/V913R/S932A | | | | |
| 245/246 | L24W/L28S/L29E/P39Q/Q50V/ | T29E | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 247/248 | L24W/L28S/L29T/P39Q/Q50V/ | E78L | + | ++ | +++ |
| | A62L/P78L/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 249/250 | L24P/L28S/L29T/P39Q/Q50V/ | W24P | +++ | +++ | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 251/252 | L24D/L28S/L29T/P39Q/Q50V/ | W24D | ++++ | ++++ | +++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 253/254 | L24W/L28S/L29T/P39Q/Q50V/ | G692K | + | + | |
| | A62L/P78E/D87E/S135Q/T150S/ | | | | |
| | T266N/R267K/A437G/T486E/E522V/ | | | | |
| | L569T/L670T/T692K/A711H/ | | | | |
| | L736M/A750P/A812E/Q830K/ | | | | |
| | G842S/L871E/R883H/Q894G/ | | | | |
| | V913R/S932A | | | | |
| 255/256 | L24W/L28S/L29D/P39Q/Q50V/ | T29D | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 257/258 | L24W/L28S/L29T/P39Q/Q50Y/ | V50Y | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 259/260 | L24W/L28S/L29T/P39Q/Q50V/ | E486K | | | + |
| | A62L/P78E/D87E/S135Q/T150S/ | | | | |
| | T266N/R267K/A437G/T486K/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 261/262 | L24W/L28F/L29T/P39Q/Q50V/ | S28F | | | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 263/264 | L24W/L28S/L29T/P39Q/Q50V/ | T569S | + | + | |
| | A62L/P78E/D87E/S135Q/T150S/ | | | | |
| | T266N/R267K/A437G/T486E/E522V/ | | | | |
| | L569S/L670T/T692G/A711H/ | | | | |
| | L736M/A750P/A812E/Q830K/ | | | | |
| | G842S/L871E/R883H/Q894G/ | | | | |
| | V913R/S932A | | | | |
| 265/266 | L24W/L28S/L29T/P39Q/Q50V/ | H883Q | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883Q/Q894G/ | | | | |
| | V913R/S932A | | | | |
| 267/268 | L24W/L28S/L29T/P39Q/Q50F/ | V50F | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 269/270 | L24W/L28W/L29T/P39Q/Q50V/ | S28W | | | +++ |
| | A62L/P78E/D87E/S135Q/T150S/ | | | | |
| | T266N/R267K/A437G/T486E/ | | | | |
| | E522V/L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 271/272 | L24W/L28S/L29T/P39Q/Q50V/ | K267V | + | + | + |
| | A62L/P78E/D87E/S135Q/T150S/ | | | | |
| | T266N/R267V/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 273/274 | L24W/L28S/L29T/P39Q/Q50V/ | K830E | | ++ | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830E/ | | | | |
| | G842S/L871E/R883H/Q894G/ | | | | |
| | V913R/S932A | | | | |
| 275/276 | L24W/L28S/L29T/P39Q/Q50V/ | L62E | + | + | |
| | A62E/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 277/278 | L24W/L28S/L29T/P39Q/Q50V/ | T569I | + | + | + |
| | A62L/P78E/D87E/S135Q/T150S/ | | | | |
| | T266N/R267K/A437G/T486E/E522V/ | | | | |
| | L569I/L670T/T692G/A711H/ | | | | |
| | L736M/A750P/A812E/Q830K/ | | | | |
| | G842S/L871E/R883H/Q894G/ | | | | |
| | V913R/S932A | | | | |
| 279/280 | L24W/L28V/L29T/P39Q/Q50V/ | S28V | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 281/282 | L24W/L28S/L29T/P39Q/Q50V/ | E486V | + | ++ | +++ |
| | A62L/P78E/D87E/S135Q/T150S/ | | | | |
| | T266N/R267K/A437G/T486V/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 283/284 | L24W/L28S/L29T/P39Q/Q50V/ | E871V | + | ++ | +++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871V/R883H/Q894G/ | | | | |
| | V913R/S932A | | | | |
| 285/286 | L24W/L28S/L29S/P39Q/Q50V/A62L/ | T29S | ++ | + | + |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 287/288 | L24W/L28C/L29T/P39Q/Q50V/ | S28C | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 289/290 | L24W/L28S/L29T/P39Q/Q50V/ | K830L | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830L/ | | | | |
| | G842S/L871E/R883H/Q894G/ | | | | |
| | V913R/S932A | | | | |
| 291/292 | L24W/L28K/L29T/P39Q/Q50V/ | S28K | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 293/294 | L24W/L28S/L29T/P39Q/Q50V/ | E871N | | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871N/R883H/Q894G/ | | | | |
| | V913R/S932A | | | | |
| 295/296 | L24W/L28S/L29T/P39Q/Q50L/A62L/ | V50L | ++ | ++ | + |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 297/298 | L24W/L28S/L29T/P39Q/Q50V/ | A932Q | ++ | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932Q | | | | |
| 299/300 | L24W/L28S/P39Q/Q50V/A62L/ | T29L | + | | ++ |
| | P78E/D87E/S135Q/T150S/T266N/ | | | | |
| | R267K/A437G/T486E/E522V/L569T/ | | | | |
| | L670T/T692G/A711H/L736M/ | | | | |
| | A750P/A812E/Q830K/G842S/ | | | | |
| | L871E/R883H/Q894G/V913R/S932A | | | | |
| 301/302 | L24W/L28S/L29T/P39Q/Q50V/ | H711F | + | + | ++ |
| | A62L/P78E/D87E/S135Q/T150S/ | | | | |
| | T266N/R267K/A437G/T486E/E522V/ | | | | |
| | L569T/L670T/T692G/A711F/ | | | | |
| | L736M/A750P/A812E/Q830K/ | | | | |
| | G842S/L871E/R883H/Q894G/ | | | | |
| | V913R/S932A | | | | |
| 303/304 | L24W/L28S/L29T/P39Q/Q50V/ | T569E | ++ | + | + |
| | A62L/P78E/D87E/S135Q/T150S/ | | | | |
| | T266N/R267K/A437G/T486E/E522V/ | | | | |
| | L569E/L670T/T692G/A711H/ | | | | |
| | L736M/A750P/A812E/Q830K/ | | | | |
| | G842S/L871E/R883H/Q894G/ | | | | |
| | V913R/S932A | | | | |
| 305/306 | L24W/L28S/L29T/P39Q/Q50V/ | E78F | | + | + |
| | A62L/P78F/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 307/308 | L24W/L28S/L29T/P39Q/Q50V/ | V522R | + | | |
| | A62L/P78E/D87E/S135Q/T150S/ | | | | |
| | T266N/R267K/A437G/T486E/E522R/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 309/310 | L24W/L28S/L29T/P39Q/Q50V/ | H711E | + | + | + |
| | A62L/P78E/D87E/S135Q/T150S/ | | | | |
| | T266N/R267K/A437G/T486E/E522V/ | | | | |
| | L569T/L670T/T692G/A711E/ | | | | |
| | L736M/A750P/A812E/Q830K/ | | | | |
| | G842S/L871E/R883H/Q894G/ | | | | |
| | V913R/S932A | | | | |
| 311/312 | L24W/L28S/L29T/P39Q/Q50V/ | S842F | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842F/L871E/R883H/Q894G/ | | | | |
| | V913R/S932A | | | | |
| 313/314 | L24W/L28S/L29T/P39Q/Q50N/ | V50N | ++ | ++ | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 315/316 | L24W/L28S/L29T/P39Q/Q50V/ | Q135D | + | + | + |
| | A62L/P78E/D87E/S135D/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 317/318 | L24W/L28S/L29T/P39Q/Q50V/ | K267R | | + | |
| | A62L/P78E/D87E/S135Q/T150S/ | | | | |
| | T266N/A437G/T486E/E522V/L569T/ | | | | |
| | L670T/T692G/A711H/L736M/ | | | | |
| | A750P/A812E/Q830K/G842S/ | | | | |
| | L871E/R883H/Q894G/V913R/S932A | | | | |
| 319/320 | L24W/L28S/L29T/P39Q/Q50V/ | E78S | + | + | +++ |
| | A62L/P78S/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 321/322 | L24V/L28S/L29T/P39Q/Q50V/A62L/ | W24V | +++ | +++ | ++ |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 323/324 | L24W/L28S/L29T/P39Q/Q50V/ | R913S | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913S/S932A | | | | |
| 325/326 | L24W/L28S/L29T/P39Q/Q50V/ | E78M | + | + | + |
| | A62L/P78M/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 327/328 | L24W/L28S/L29T/P39Q/Q50R/A62L/ | V50R | ++ | ++ | ++ |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 329/330 | L24W/L28S/L29T/P39Q/Q50V/ | T670Q | + | + | |
| | A62L/P78E/D87E/S135Q/T150S/ | | | | |
| | T266N/R267K/A437G/T486E/ | | | | |
| | E522V/L569T/L670Q/T692G/A711H/ | | | | |
| | L736M/A750P/A812E/Q830K/ | | | | |
| | G842S/L871E/R883H/Q894G/ | | | | |
| | V913R/S932A | | | | |
| 331/332 | L24W/L28S/L29T/P39Q/Q50V/ | L62T | | + | |
| | A62T/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 333/334 | L24W/L28S/L29T/P39Q/Q50V/ | G894M | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894M/ | | | | |
| | V913R/S932A | | | | |
| 335/336 | L24W/L28S/L29T/P39Q/Q50K/ | V50K | ++ | ++ | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 337/338 | L24W/L28S/L29T/P39Q/Q50V/ | E78N | + | + | + |
| | A62L/P78N/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 339/340 | L24W/L28S/L29T/P39Q/Q50V/ | P750L | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750L/A812E/Q830K/ | | | | |
| | G842S/L871E/R883H/Q894G/ | | | | |
| | V913R/S932A | | | | |
| 341/342 | L24W/L28S/L29T/P39Q/Q50V/ | P750R | | | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750R/A812E/Q830K/ | | | | |
| | G842S/L871E/R883H/Q894G/ | | | | |
| | V913R/S932A | | | | |
| 343/344 | L24W/L28S/L29T/P39Q/Q50V/ | T569D | | + | |
| | A62L/P78E/D87E/S135Q/T150S/ | | | | |
| | T266N/R267K/A437G/T486E/E522V/ | | | | |
| | L569D/L670T/T692G/A711H/ | | | | |
| | L736M/A750P/A812E/Q830K/ | | | | |
| | G842S/L871E/R883H/Q894G/ | | | | |
| | V913R/S932A | | | | |
| 345/346 | L24I/L28S/L29T/P39Q/Q50V/A62L/ | W24I | +++ | ++ | + |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 347/348 | L24W/L28S/L29T/P39Q/Q50V/ | E486N | + | ++ | +++ |
| | A62L/P78E/D87E/S135Q/T150S/ | | | | |
| | T266N/R267K/A437G/T486N/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 349/350 | L24W/L28S/L29T/P39Q/Q50V/ | G894V | + | ++ | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894V/ | | | | |
| | V913R/S932A | | | | |
| 351/352 | L24W/L28S/L29T/P39Q/Q50V/ | K830D | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830D/ | | | | |
| | G842S/L871E/R883H/Q894G/ | | | | |
| | V913R/S932A | | | | |
| 353/354 | L24W/L28S/L29T/P39Q/Q50V/ | G894W | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894W/ | | | | |
| | V913R/S932A | | | | |
| 355/356 | L24W/L28S/L29T/P39Q/Q50V/ | E78V | + | + | + |
| | A62L/P78V/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 357/358 | L24W/L28S/L29T/P39Q/Q50V/ | V522A | + | | + |
| | A62L/P78E/D87E/S135Q/T150S/ | | | | |
| | T266N/R267K/A437G/T486E/E522A/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 359/360 | L24W/L28S/L29T/P39Q/Q50V/ | T670M | + | + | |
| | A62L/P78E/D87E/S135Q/T150S/ | | | | |
| | T266N/R267K/A437G/T486E/E522V/ | | | | |
| | L569T/L670M/T692G/A711H/ | | | | |
| | L736M/A750P/A812E/Q830K/ | | | | |
| | G842S/L871E/R883H/Q894G/ | | | | |
| | V913R/S932A | | | | |
| 361/362 | L24W/L28S/L29T/P39Q/Q50V/ | L62Y | | + | |
| | A62Y/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 363/364 | L24W/L29T/P39Q/Q50V/A62L/ | S28L | + | | + |
| | P78E/D87E/S135Q/T150S/T266N/ | | | | |
| | R267K/A437G/T486E/E522V/ | | | | |
| | L569T/L670T/T692G/A711H/L736M/ | | | | |
| | A750P/A812E/Q830K/G842S/ | | | | |
| | L871E/R883H/Q894G/V913R/ | | | | |
| | S932A | | | | |
| 365/366 | L24W/L28S/L29T/P39Q/Q50V/ | P750K | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750K/A812E/Q830K/ | | | | |
| | G842S/L871E/R883H/Q894G/ | | | | |
| | V913R/S932A | | | | |
| 367/368 | L24W/L28S/L29T/P39Q/Q50V/ | E78A | + | ++ | +++ |
| | A62L/P78A/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 369/370 | L24W/L28S/L29T/P39Q/Q50G/ | V50G | ++ | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 371/372 | L24W/L28S/L29T/P39Q/Q50V/ | G894K | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894K/ | | | | |
| | V913R/S932A | | | | |
| 373/374 | L24W/L28S/L29T/P39Q/Q50V/ | G437S | + | + | |
| | A62L/P78E/D87E/S135Q/T150S/ | | | | |
| | T266N/R267K/A437S/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 375/376 | L24W/L28S/L29T/P39Q/Q50V/ | G692E | | + | |
| | A62L/P78E/D87E/S135Q/T150S/ | | | | |
| | T266N/R267K/A437G/T486E/E522V/ | | | | |
| | L569T/L670T/T692E/A711H/ | | | | |
| | L736M/A750P/A812E/Q830K/ | | | | |
| | G842S/L871E/R883H/Q894G/ | | | | |
| | V913R/S932A | | | | |
| 377/378 | L24W/L28S/L29T/P39Q/Q50V/ | T569P | | + | |
| | A62L/P78E/D87E/S135Q/T150S/ | | | | |
| | T266N/R267K/A437G/T486E/E522V/ | | | | |
| | L569P/L670T/T692G/A711H/ | | | | |
| | L736M/A750P/A812E/Q830K/ | | | | |
| | G842S/L871E/R883H/Q894G/ | | | | |
| | V913R/S932A | | | | |
| 379/380 | L24W/L28S/L29T/P39Q/Q50V/ | T569N | | + | |
| | A62L/P78E/D87E/S135Q/T150S/ | | | | |
| | T266N/R267K/A437G/T486E/ | | | | |
| | E522V/L569N/L670T/T692G/ | | | | |
| | A711H/L736M/A750P/A812E/Q830K/ | | | | |
| | G842S/L871E/R883H/Q894G/ | | | | |
| | V913R/S932A | | | | |
| 381/382 | L24W/L28S/L29T/P39Q/Q50V/ | E87D | + | + | + |
| | A62L/P78E/S135Q/T150S/T266N/ | | | | |
| | R267K/A437G/T486E/E522V/ | | | | |
| | L569T/L670T/T692G/A711H/L736M/ | | | | |
| | A750P/A812E/Q830K/G842S/ | | | | |
| | L871E/R883H/Q894G/V913R/ | | | | |
| | S932A | | | | |
| 383/384 | L24W/L28S/L29T/P39Q/Q50V/ | Q135N | | + | |
| | A62L/P78E/D87E/S135N/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 385/386 | L24W/L28S/L29T/P39Q/Q50V/ | V522Y | + | | |
| | A62L/P78E/D87E/S135Q/T150S/ | | | | |
| | T266N/R267K/A437G/T486E/E522Y/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 387/388 | L24W/L28S/L29T/P39Q/Q50V/ | G437A | + | + | ++ |
| | A62L/P78E/D87E/S135Q/T150S/ | | | | |
| | T266N/R267K/T486E/E522V/L569T/ | | | | |
| | L670T/T692G/A711H/L736M/ | | | | |
| | A750P/A812E/Q830K/G842S/ | | | | |
| | L871E/R883H/Q894G/V913R/S932A | | | | |
| 389/390 | L24W/L28S/L29T/P39Q/Q50V/ | M736F | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736F/A750P/A812E/Q830K/ | | | | |
| | G842S/L871E/R883H/Q894G/ | | | | |
| | V913R/S932A | | | | |
| 391/392 | L24W/L28S/L29T/P39Q/Q50V/ | A932T | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932T | | | | |
| 393/394 | L24G/L28S/L29T/P39Q/Q50V/A62L/ | W24G | ++++ | +++ | +++ |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 395/396 | L24W/L28S/L29T/P39Q/Q50V/ | S842K | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842K/L871E/R883H/Q894G/ | | | | |
| | V913R/S932A | | | | |
| 397/398 | L24W/L28S/L29T/P39Q/Q50V/ | A932W | | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932W | | | | |
| 399/400 | L24W/L28S/L29T/P39Q/Q50V/ | G692Q | + | + | |
| | A62L/P78E/D87E/S135Q/T150S/ | | | | |
| | T266N/R267K/A437G/T486E/E522V/ | | | | |
| | L569T/L670T/T692Q/A711H/ | | | | |
| | L736M/A750P/A812E/Q830K/ | | | | |
| | G842S/L871E/R883H/Q894G/ | | | | |
| | V913R/S932A | | | | |
| 401/402 | L24W/L28S/L29T/P39Q/Q50V/ | Q135F | + | + | |
| | A62L/P78E/D87E/S135F/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 403/404 | L24W/L28S/L29T/P39Q/Q50V/ | E78P | + | ++ | +++ |
| | A62L/D87E/S135Q/T150S/T266N/ | | | | |
| | R267K/A437G/T486E/E522V/ | | | | |
| | L569T/L670T/T692G/A711H/L736M/ | | | | |
| | A750P/A812E/Q830K/G842S/ | | | | |
| | L871E/R883H/Q894G/V913R/S932A | | | | |
| 405/406 | L24W/L28S/L29T/P39Q/Q50V/ | E871F | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871F/R883H/Q894G/ | | | | |
| | V913R/S932A | | | | |
| 407/408 | L24W/L28S/L29T/P39Q/Q50V/ | T670Y | + | + | ++ |
| | A62L/P78E/D87E/S135Q/T150S/ | | | | |
| | T266N/R267K/A437G/T486E/E522V/ | | | | |
| | L569T/L670Y/T692G/A711H/ | | | | |
| | L736M/A750P/A812E/Q830K/ | | | | |
| | G842S/L871E/R883H/Q894G/ | | | | |
| | V913R/S932A | | | | |
| 409/410 | L24W/L28S/L29T/P39Q/Q50V/ | G692L | + | + | +++ |
| | A62L/P78E/D87E/S135Q/T150S/ | | | | |
| | T266N/R267K/A437G/T486E/E522V/ | | | | |
| | L569T/L670T/T692L/A711H/ | | | | |
| | L736M/A750P/A812E/Q830K/ | | | | |
| | G842S/L871E/R883H/Q894G/ | | | | |
| | V913R/S932A | | | | |
| 411/412 | L24W/L28S/L29T/P39Q/Q50V/ | Q135E | + | + | |
| | A62L/P78E/D87E/S135E/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 413/414 | L24W/L28S/L29T/P39Q/Q50V/ | T670F | + | + | |
| | A62L/P78E/D87E/S135Q/T150S/ | | | | |
| | T266N/R267K/A437G/T486E/E522V/ | | | | |
| | L569T/L670F/T692G/A711H/ | | | | |
| | L736M/A750P/A812E/Q830K/ | | | | |
| | G842S/L871E/R883H/Q894G/ | | | | |
| | V913R/S932A | | | | |
| 415/416 | L24W/L28S/L29T/P39Q/Q50H/ | V50H | ++ | ++ | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 417/418 | L24W/L28S/L29T/P39Q/Q50V/ | E87I | | | + |
| | A62L/P78E/D87I/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 419/420 | L24W/L28P/L29T/P39Q/Q50V/ | S28P | | | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 421/422 | L24W/L28S/L29T/P39Q/Q50V/ | H883F | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883F/Q894G/ | | | | |
| | V913R/S932A | | | | |
| 423/424 | L24W/L28S/L29T/P39Q/Q50V/ | G437H | + | + | |
| | A62L/P78E/D87E/S135Q/T150S/ | | | | |
| | T266N/R267K/A437H/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 425/426 | L24W/L28S/L29T/P39Q/Q50V/ | L62I | | + | |
| | A62I/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 427/428 | L24W/L28S/L29T/P39Q/Q50V/ | K830W | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830W/ | | | | |
| | G842S/L871E/R883H/Q894G/ | | | | |
| | V913R/S932A | | | | |
| 429/430 | L24W/L28S/L29T/P39Q/Q50V/ | G692S | + | + | |
| | A62L/P78E/D87E/S135Q/T150S/ | | | | |
| | T266N/R267K/A437G/T486E/E522V/ | | | | |
| | L569T/L670T/T692S/A711H/ | | | | |
| | L736M/A750P/A812E/Q830K/ | | | | |
| | G842S/L871E/R883H/Q894G/ | | | | |
| | V913R/S932A | | | | |
| 431/432 | L24W/L28S/L29K/P39Q/Q50V/ | T29K | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 433/434 | L24W/L28S/L29T/P39Q/Q50V/ | T670E | + | + | |
| | A62L/P78E/D87E/S135Q/T150S/ | | | | |
| | T266N/R267K/A437G/T486E/E522V/ | | | | |
| | L569T/L670E/T692G/A711H/ | | | | |
| | L736M/A750P/A812E/Q830K/ | | | | |
| | G842S/L871E/R883H/Q894G/ | | | | |
| | V913R/S932A | | | | |
| 435/436 | L24W/L28S/L29T/P39Q/Q50V/ | E486R | + | + | ++ |
| | A62L/P78E/D87E/S135Q/T150S/ | | | | |
| | T266N/R267K/A437G/T486R/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 437/438 | L24W/L28S/L29T/P39Q/Q50V/ | Q135A | + | + | ++ |
| | A62L/P78E/D87E/S135A/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 439/440 | L24W/L28S/L29T/P39I/Q50V/A62L/ | Q39I | | + | |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 441/442 | L24W/L28S/L29T/P39Q/Q50V/ | E87N | | + | ++ |
| | A62L/P78E/D87N/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 443/444 | L24W/L28S/L29T/P39Q/Q50V/ | E87M | + | + | ++ |
| | A62L/P78E/D87M/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 445/446 | L24W/L28S/L29T/P39Q/Q50V/ | Q135C | + | + | |
| | A62L/P78E/D87E/S135C/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 447/448 | L24W/L28S/L29T/P39Q/Q50V/ | A932L | | | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932L | | | | |
| 449/450 | L24W/L28S/L29T/P39Q/Q50V/ | T670G | + | + | |
| | A62L/P78E/D87E/S135Q/T150S/ | | | | |
| | T266N/R267K/A437G/T486E/E522V/ | | | | |
| | L569T/L670G/T692G/A711H/ | | | | |
| | L736M/A750P/A812E/Q830K/ | | | | |
| | G842S/L871E/R883H/Q894G/ | | | | |
| | V913R/S932A | | | | |
| 451/452 | L24W/L28S/L29T/P39Q/Q50V/ | K830F | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830F/ | | | | |
| | G842S/L871E/R883H/Q894G/ | | | | |
| | V913R/S932A | | | | |
| 453/454 | L24W/L28S/L29T/P39Q/Q50V/ | V522H | + | | |
| | A62L/P78E/D87E/S135Q/T150S/ | | | | |
| | T266N/R267K/A437G/T486E/E522H/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 455/456 | L24W/L28D/L29T/P39Q/Q50V/ | S28D | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 457/458 | L24W/L28S/L29T/P39Q/Q50V/ | V522Q | ++ | ++ | + |
| | A62L/P78E/D87E/S135Q/T150S/ | | | | |
| | T266N/R267K/A437G/T486E/E522Q/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 459/460 | L24W/L28S/L29T/P39Q/Q50A/ | V50A | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 461/462 | L24W/L28S/L29T/P39Q/Q50V/ | T670S | ++ | ++ | + |
| | A62L/P78E/D87E/S135Q/T150S/ | | | | |
| | T266N/R267K/A437G/T486E/E522V/ | | | | |
| | L569T/L670S/T692G/A711H/ | | | | |
| | L736M/A750P/A812E/Q830K/ | | | | |
| | G842S/L871E/R883H/Q894G/ | | | | |
| | V913R/S932A | | | | |
| 463/464 | L24W/L28S/L29T/P39Q/Q50V/ | K830M | + | ++ | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830M/ | | | | |
| | G842S/L871E/R883H/Q894G/ | | | | |
| | V913R/S932A | | | | |
| 465/466 | L24W/L28S/L29T/P39Q/Q50V/ | S842A | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842A/L871E/R883H/Q894G/ | | | | |
| | V913R/S932A | | | | |
| 467/468 | L24W/L28S/L29T/P39Q/Q50V/ | E87T | | + | ++ |
| | A62L/P78E/D87T/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 469/470 | L24W/L28S/L29T/P39Q/Q50V/ | V522T | + | + | |
| | A62L/P78E/D87E/S135Q/T150S/ | | | | |
| | T266N/R267K/A437G/T486E/E522T/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 471/472 | L24K/L28S/L29T/P39Q/Q50V/A62L/ | W24K | +++ | +++ | ++ |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 473/474 | L24W/L28S/L29T/P39Q/Q50V/ | E871K | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871K/R883H/Q894G/ | | | | |
| | V913R/S932A | | | | |
| 475/476 | L24W/L28S/L29T/P39Q/Q50V/ | P750E | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750E/A812E/Q830K/ | | | | |
| | G842S/L871E/R883H/Q894G/ | | | | |
| | V913R/S932A | | | | |
| 477/478 | L24W/L28S/L29T/P39Q/Q50V/ | K830T | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830T/ | | | | |
| | G842S/L871E/R883H/Q894G/ | | | | |
| | V913R/S932A | | | | |
| 479/480 | L24W/L28S/L29T/P39Q/Q50I/A62L/ | V50I | ++ | ++ | + |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 481/482 | L24W/L28S/L29T/P39Q/Q50V/ | A932E | ++ | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932E | | | | |
| 483/484 | L24W/L28S/L29T/P39Q/Q50V/ | N266Q | + | + | |
| | A62L/P78E/D87E/S135Q/T150S/ | | | | |
| | T266Q/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 485/486 | L24W/L28S/L29T/P39A/Q50V/ | Q39A | | | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 487/488 | L24T/L28S/L29T/P39Q/Q50V/A62L/ | W24T | +++ | +++ | ++ |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 489/490 | L24W/L28S/L29T/P39Q/Q50V/ | E871T | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871T/R883H/Q894G/ | | | | |
| | V913R/S932A | | | | |
| 491/492 | L24W/L28S/L29T/P39Q/Q50V/ | S842M | + | | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842M/L871E/R883H/Q894G/ | | | | |
| | V913R/S932A | | | | |
| 493/494 | L24W/L28S/L29T/P39Q/Q50V/ | H711T | + | ++ | + |
| | A62L/P78E/D87E/S135Q/T150S/ | | | | |
| | T266N/R267K/A437G/T486E/E522V/ | | | | |
| | L569T/L670T/T692G/A711T/ | | | | |
| | L736M/A750P/A812E/Q830K/ | | | | |
| | G842S/L871E/R883H/Q894G/ | | | | |
| | V913R/S932A | | | | |
| 495/496 | L24W/L28S/L29T/P39Q/Q50V/ | N266K | + | | |
| | A62L/P78E/D87E/S135Q/T150S/ | | | | |
| | T266K/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 497/498 | L24W/L28S/L29T/P39Q/Q50V/ | E486F | + | + | + |
| | A62L/P78E/D87E/S135Q/T150S/ | | | | |
| | T266N/R267K/A437G/T486F/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 499/500 | L24W/L28S/L29T/P39Q/Q50M/ | V50M | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 501/502 | L24W/L28S/L29T/P39Q/Q50V/ | E486Y | + | + | ++ |
| | A62L/P78E/D87E/S135Q/T150S/ | | | | |
| | T266N/R267K/A437G/T486Y/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 503/504 | L24W/L28S/L29T/P39Q/Q50V/ | T670R | + | + | ++ |
| | A62L/P78E/D87E/S135Q/T150S/ | | | | |
| | T266N/R267K/A437G/T486E/E522V/ | | | | |
| | L569T/L670R/T692G/A711H/ | | | | |
| | L736M/A750P/A812E/Q830K/ | | | | |
| | G842S/L871E/R883H/Q894G/ | | | | |
| | V913R/S932A | | | | |
| 505/506 | L24M/L28S/L29T/P39Q/Q50V/A62L/ | W24M | +++ | +++ | ++ |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 507/508 | L24W/L28S/L29T/P39Q/Q50V/ | L62F | + | + | + |
| | A62F/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 509/510 | L24W/L28S/L29T/P39Q/Q50V/ | T569Y | + | + | |
| | A62L/P78E/D87E/S135Q/T150S/ | | | | |
| | T266N/R267K/A437G/T486E/E522V/ | | | | |
| | L569Y/L670T/T692G/A711H/ | | | | |
| | L736M/A750P/A812E/Q830K/ | | | | |
| | G842S/L871E/R883H/Q894G/ | | | | |
| | V913R/S932A | | | | |
| 511/512 | L24W/L28S/L29T/P39Q/Q50V/ | H711Q | | ++ | |
| | A62L/P78E/D87E/S135Q/T150S/ | | | | |
| | T266N/R267K/A437G/T486E/E522V/ | | | | |
| | L569T/L670T/T692G/A711Q/ | | | | |
| | L736M/A750P/A812E/Q830K/ | | | | |
| | G842S/L871E/R883H/Q894G/ | | | | |
| | V913R/S932A | | | | |
| 513/514 | L24W/L28S/L29T/P39Q/Q50V/ | E486I | + | + | + |
| | A62L/P78E/D87E/S135Q/T150S/ | | | | |
| | T266N/R267K/A437G/T486I/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 515/516 | L24W/L28S/L29T/P39Q/Q50V/ | Q135H | + | + | + |
| | A62L/P78E/D87E/S135H/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 517/518 | L24W/L28S/L29T/P39Q/Q50V/ | T670H | + | + | |
| | A62L/P78E/D87E/S135Q/T150S/ | | | | |
| | T266N/R267K/A437G/T486E/E522V/ | | | | |
| | L569T/L670H/T692G/A711H/ | | | | |
| | L736M/A750P/A812E/Q830K/ | | | | |
| | G842S/L871E/R883H/Q894G/ | | | | |
| | V913R/S932A | | | | |
| 519/520 | L24W/L28S/L29T/P39Q/Q50V/ | V522N | + | | |
| | A62L/P78E/D87E/S135Q/T150S/ | | | | |
| | T266N/R267K/A437G/T486E/E522N/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 521/522 | L24W/L28S/L29T/P39Q/Q50V/ | G894E | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894E/ | | | | |
| | V913R/S932A | | | | |
| 523/524 | L24W/L28S/L29T/P39Q/Q50V/ | E871Q | + | ++ | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871Q/R883H/Q894G/ | | | | |
| | V913R/S932A | | | | |
| 525/526 | L24W/L28E/L29T/P39Q/Q50V/ | S28E | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 527/528 | L24W/L28S/L29T/P39Q/Q50V/ | S842N | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842N/L871E/R883H/Q894G/ | | | | |
| | V913R/S932A | | | | |
| 529/530 | L24W/L28S/L29T/P39Q/Q50V/ | E78H | + | + | ++ |
| | A62L/P78H/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 531/532 | L24W/L28S/L29T/P39Q/Q50V/ | S150T | + | + | + |
| | A62L/P78E/D87E/S135Q/T266N/ | | | | |
| | R267K/A437G/T486E/E522V/L569T/ | | | | |
| | L670T/T692G/A711H/L736M/ | | | | |
| | A750P/A812E/Q830K/G842S/ | | | | |
| | L871E/R883H/Q894G/V913R/ | | | | |
| | S932A | | | | |
| 533/534 | L24W/L28S/L29T/P39Q/Q50V/ | K830Q | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/G842S/ | | | | |
| | L871E/R883H/Q894G/V913R/S932A | | | | |
| 535/536 | L24W/L28S/L29T/P39Q/Q50V/ | R913I | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913I/S932A | | | | |
| 537/538 | L24W/L28S/L29T/P39Q/Q50V/ | R913H | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913H/S932A | | | | |
| 539/540 | L24W/L28S/L29T/P39Q/Q50V/ | E871Y | + | ++ | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871Y/R883H/Q894G/ | | | | |
| | V913R/S932A | | | | |
| 541/542 | L24W/L28S/L29N/P39Q/Q50V/ | T29N | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 543/544 | L24W/L28S/L29T/P39Q/Q50V/ | T670K | + | + | + |
| | A62L/P78E/D87E/S135Q/T150S/ | | | | |
| | T266N/R267K/A437G/T486E/E522V/ | | | | |
| | L569T/L670K/T692G/A711H/ | | | | |
| | L736M/A750P/A812E/Q830K/ | | | | |
| | G842S/L871E/R883H/Q894G/ | | | | |
| | V913R/S932A | | | | |
| 545/546 | L24W/L28S/L29T/P39Q/Q50V/ | G692A | + | + | + |
| | A62L/P78E/D87E/S135Q/T150S/ | | | | |
| | T266N/R267K/A437G/T486E/E522V/ | | | | |
| | L569T/L670T/T692A/A711H/ | | | | |
| | L736M/A750P/A812E/Q830K/ | | | | |
| | G842S/L871E/R883H/Q894G/ | | | | |
| | V913R/S932A | | | | |
| 547/548 | L24W/L28S/L29T/P39Q/Q50T/A62L/ | V50T | + | + | |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 549/550 | L24W/L28S/L29T/P39Q/Q50V/ | S842H | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842H/L871E/R883H/Q894G/ | | | | |
| | V913R/S932A | | | | |
| 551/552 | L24W/L28S/L29T/P39Q/Q50V/ | G692M | + | + | |
| | A62L/P78E/D87E/S135Q/T150S/ | | | | |
| | T266N/R267K/A437G/T486E/E522V/ | | | | |
| | L569T/L670T/T692M/A711H/ | | | | |
| | L736M/A750P/A812E/Q830K/ | | | | |
| | G842S/L871E/R883H/Q894G/ | | | | |
| | V913R/S932A | | | | |
| 553/554 | L24W/L28S/L29Q/P39Q/Q50V/ | T29Q | ++ | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 555/556 | L24W/L28S/L29T/P39Q/Q50V/ | G894D | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894D/ | | | | |
| | V913R/S932A | | | | |
| 557/558 | L24W/L28S/L29T/P39Q/Q50V/ | R913F | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913F/S932A | | | | |
| 559/560 | L24W/L28S/L29T/P39Q/Q50V/ | T670D | ++ | ++ | |
| | A62L/P78E/D87E/S135Q/T150S/ | | | | |
| | T266N/R267K/A437G/T486E/E522V/ | | | | |
| | L569T/L670D/T692G/A711H/ | | | | |
| | L736M/A750P/A812E/Q830K/ | | | | |
| | G842S/L871E/R883H/Q894G/ | | | | |
| | V913R/S932A | | | | |
| 561/562 | L24W/L28S/L29T/P39Q/Q50V/ | L62M | + | + | ++ |
| | A62M/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 563/564 | L24W/L28S/L29T/P39Q/Q50V/ | E486C | | | + |
| | A62L/P78E/D87E/S135Q/T150S/ | | | | |
| | T266N/R267K/A437G/T486C/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 565/566 | L24W/L28S/L29T/P39Q/Q50V/ | E78T | | + | + |
| | A62L/P78T/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 567/568 | L24W/L28S/L29T/P39Q/Q50V/ | E812A | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/Q830K/G842S/ | | | | |
| | L871E/R883H/Q894G/V913R/S932A | | | | |
| 569/570 | L24W/L28S/L29T/P39Q/Q50V/ | K267H | | + | |
| | A62L/P78E/D87E/S135Q/T150S/ | | | | |
| | T266N/R267H/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 571/572 | L24W/L28S/L29I/P39Q/Q50V/A62L/ | T29I | + | | |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 573/574 | L24H/L28S/L29T/P39Q/Q50V/A62L/ | W24H | ++ | + | |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 575/576 | L24W/L28S/L29T/P39Q/Q50V/ | Q135I | + | + | ++ |
| | A62L/P78E/D87E/S135I/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 577/578 | L24W/L28S/L29T/P39Q/Q50V/ | G692H | + | + | ++ |
| | A62L/P78E/D87E/S135Q/T150S/ | | | | |
| | T266N/R267K/A437G/T486E/E522V/ | | | | |
| | L569T/L670T/T692H/A711H/ | | | | |
| | L736M/A750P/A812E/Q830K/ | | | | |
| | G842S/L871E/R883H/Q894G/ | | | | |
| | V913R/S932A | | | | |
| 579/580 | L24W/L28H/L29T/P39Q/Q50V/ | S28H | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 581/582 | L24W/L28S/L29T/P39Q/Q50V/ | R913N | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913N/S932A | | | | |
| 583/584 | L24W/L28S/L29T/P39Q/Q50V/ | S842D | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842D/L871E/R883H/Q894G/ | | | | |
| | V913R/S932A | | | | |
| 585/586 | L24W/L28S/L29T/P39Q/Q50V/ | V522K | + | | |
| | A62L/P78E/D87E/S135Q/T150S/ | | | | |
| | T266N/R267K/A437G/T486E/E522K/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 587/588 | L24W/L28S/L29T/P39Q/Q50V/ | G894H | + | ++ | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894H/ | | | | |
| | V913R/S932A | | | | |
| 589/590 | L24W/L28S/L29T/P39Q/Q50V/ | E78K | + | + | ++ |
| | A62L/P78K/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 591/592 | L24W/L28S/L29T/P39F/Q50V/A62L/ | Q39F | + | + | |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 593/594 | L24W/L28S/L29T/P39Q/Q50V/ | E871W | + | ++ | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871W/R883H/Q894G/ | | | | |
| | V913R/S932A | | | | |
| 595/596 | L24W/L28S/L29T/P39Q/Q50V/ | Q135Y | | | + |
| | A62L/P78E/D87E/S135Y/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 597/598 | L24W/L28S/L29T/P39Q/Q50V/ | S842Q | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842Q/L871E/R883H/Q894G/ | | | | |
| | V913R/S932A | | | | |
| 599/600 | L24W/L28S/L29H/P39Q/Q50V/ | T29H | + | | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 601/602 | L24W/L28S/L29T/P39Q/Q50V/ | E78Q | + | + | ++ |
| | A62L/P78Q/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 603/604 | L24W/L28S/L29T/P39Q/Q50V/ | V522D | + | | |
| | A62L/P78E/D87E/S135Q/T150S/ | | | | |
| | T266N/R267K/A437G/T486E/E522D/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 605/606 | L24W/L28S/L29T/P39Q/Q50V/ | A932Y | + | + | +++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932Y | | | | |
| 607/608 | L24W/L28S/L29V/P39Q/Q50V/ | T29V | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 609/610 | L24W/L28S/L29T/P39Q/A62L/P78E/ | V50Q | + | + | |
| | D87E/S135Q/T150S/T266N/ | | | | |
| | R267K/A437G/T486E/E522V/L569T/ | | | | |
| | L670T/T692G/A711H/L736M/ | | | | |
| | A750P/A812E/Q830K/G842S/ | | | | |
| | L871E/R883H/Q894G/V913R/S932A | | | | |
| 611/612 | L24W/L28S/L29T/P39Q/Q50V/ | S842L | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842L/L871E/R883H/Q894G/ | | | | |
| | V913R/S932A | | | | |
| 613/614 | L24W/L28S/L29T/P39Q/Q50V/ | G692F | + | ++ | |
| | A62L/P78E/D87E/S135Q/T150S/ | | | | |
| | T266N/R267K/A437G/T486E/E522V/ | | | | |
| | L569T/L670T/T692F/A711H/ | | | | |
| | L736M/A750P/A812E/Q830K/ | | | | |
| | G842S/L871E/R883H/Q894G/ | | | | |
| | V913R/S932A | | | | |
| 615/616 | L24W/L28S/L29R/P39Q/Q50V/ | T29R | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 617/618 | L24W/L28S/L29T/P39Q/Q50V/ | G894I | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894I/ | | | | |
| | V913R/S932A | | | | |
| 619/620 | L24W/L28S/L29T/P39N/Q50V/ | Q39N | | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 621/622 | L24W/L28S/L29T/P39Q/Q50V/ | G692T | | + | |
| | A62L/P78E/D87E/S135Q/T150S/ | | | | |
| | T266N/R267K/A437G/T486E/E522V/ | | | | |
| | L569T/L670T/A711H/L736M/ | | | | |
| | A750P/A812E/Q830K/G842S/ | | | | |
| | L871E/R883H/Q894G/V913R/S932A | | | | |
| 623/624 | L24W/L28S/L29T/P39Q/Q50V/ | N266E | + | + | |
| | A62L/P78E/D87E/S135Q/T150S/ | | | | |
| | T266E/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 625/626 | L24W/L28S/L29T/P39Q/Q50V/ | E78C | | + | + |
| | A62L/P78C/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 627/628 | L24W/L28S/L29T/P39Q/Q50V/ | G894Q | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/V913R/S932A | | | | |
| 629/630 | L24W/L28S/L29T/P39Q/Q50V/ | A932P | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932P | | | | |
| 631/632 | L24W/L28S/L29T/P39Q/Q50V/ | A932D | ++ | ++ | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932D | | | | |
| 633/634 | L24W/L28S/L29T/P39Q/Q50V/ | K830Y | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830Y/ | | | | |
| | G842S/L871E/R883H/Q894G/ | | | | |
| | V913R/S932A | | | | |
| 635/636 | L24W/L28S/L29T/P39Q/Q50V/ | Q135K | + | + | + |
| | A62L/P78E/D87E/S135K/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 637/638 | L24W/L28S/L29T/P39Q/Q50V/ | G692D | + | ++ | |
| | A62L/P78E/D87E/S135Q/T150S/ | | | | |
| | T266N/R267K/A437G/T486E/E522V/ | | | | |
| | L569T/L670T/T692D/A711H/ | | | | |
| | L736M/A750P/A812E/Q830K/ | | | | |
| | G842S/L871E/R883H/Q894G/ | | | | |
| | V913R/S932A | | | | |
| 639/640 | L24W/L28G/L29T/P39Q/Q50V/ | S28G | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 641/642 | L24W/L28S/L29T/P39Q/Q50V/ | H711N | + | ++ | + |
| | A62L/P78E/D87E/S135Q/T150S/ | | | | |
| | T266N/R267K/A437G/T486E/E522V/ | | | | |
| | L569T/L670T/T692G/A711N/ | | | | |
| | L736M/A750P/A812E/Q830K/ | | | | |
| | G842S/L871E/R883H/Q894G/ | | | | |
| | V913R/S932A | | | | |
| 643/644 | L24W/L28S/L29T/P39Q/Q50V/ | E486W | | | ++ |
| | A62L/P78E/D87E/S135Q/T150S/ | | | | |
| | T266N/R267K/A437G/T486W/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 645/646 | L24W/L28S/L29T/P39Q/Q50D/ | V50D | + | ++ | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 647/648 | L24W/L28S/L29T/P39Q/Q50V/ | E486A | + | + | ++ |
| | A62L/P78E/D87E/S135Q/T150S/ | | | | |
| | T266N/R267K/A437G/T486A/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 649/650 | L24W/L28S/L29T/P39Q/Q50V/ | G894N | + | ++ | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894N/ | | | | |
| | V913R/S932A | | | | |
| 651/652 | L24W/L28S/L29T/P39Q/Q50V/ | A932G | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932G | | | | |
| 653/654 | L24W/L28S/L29T/P39Q/Q50V/ | H711D | + | + | |
| | A62L/P78E/D87E/S135Q/T150S/ | | | | |
| | T266N/R267K/A437G/T486E/E522V/ | | | | |
| | L569T/L670T/T692G/A711D/ | | | | |
| | L736M/A750P/A812E/Q830K/ | | | | |
| | G842S/L871E/R883H/Q894G/ | | | | |
| | V913R/S932A | | | | |
| 655/656 | L24W/L28S/L29T/P39Q/Q50V/ | N266T | + | | |
| | A62L/P78E/D87E/S135Q/T150S/ | | | | |
| | R267K/A437G/T486E/E522V/L569T/ | | | | |
| | L670T/T692G/A711H/L736M/ | | | | |
| | A750P/A812E/Q830K/G842S/ | | | | |
| | L871E/R883H/Q894G/V913R/S932A | | | | |
| 657/658 | L24W/L28S/L29T/P39Q/Q50V/ | E871H | ++ | ++ | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871H/R883H/Q894G/ | | | | |
| | V913R/S932A | | | | |
| 659/660 | L24W/L28S/L29T/P39Q/Q50V/ | E486H | + | + | +++ |
| | A62L/P78E/D87E/S135Q/T150S/ | | | | |
| | T266N/R267K/A437G/T486H/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 661/662 | L24W/L28S/L29T/P39Q/Q50V/ | R913K | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913K/S932A | | | | |
| 663/664 | L24W/L28S/L29T/P39Q/Q50V/ | E871M | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871M/R883H/Q894G/ | | | | |
| | V913R/S932A | | | | |
| 665/666 | L24W/L28S/L29T/P39E/Q50V/A62L/ | Q39E | + | + | |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 667/668 | L24W/L28S/L29T/P39Q/Q50V/ | A932N | ++ | ++ | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932N | | | | |
| 669/670 | L24W/L28S/L29T/P39Q/Q50V/ | A932M | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932M | | | | |
| 671/672 | L24W/L28S/L29T/P39Q/Q50V/ | R913M | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913M/S932A | | | | |
| 673/674 | L24W/L28S/L29T/P39Q/Q50V/ | T569W | + | + | ++ |
| | A62L/P78E/D87E/S135Q/T150S/ | | | | |
| | T266N/R267K/A437G/T486E/ | | | | |
| | E522V/L569W/L670T/T692G/A711H/ | | | | |
| | L736M/A750P/A812E/Q830K/ | | | | |
| | G842S/L871E/R883H/Q894G/ | | | | |
| | V913R/S932A | | | | |
| 675/676 | L24W/L28S/L29T/P39Q/Q50V/ | E486S | + | + | + |
| | A62L/P78E/D87E/S135Q/T150S/ | | | | |
| | T266N/R267K/A437G/T486S/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 677/678 | L24W/L28S/L29T/P39Q/Q50V/ | H711I | + | + | |
| | A62L/P78E/D87E/S135Q/T150S/ | | | | |
| | T266N/R267K/A437G/T486E/E522V/ | | | | |
| | L569T/L670T/T692G/A711I/ | | | | |
| | L736M/A750P/A812E/Q830K/ | | | | |
| | G842S/L871E/R883H/Q894G/ | | | | |
| | V913R/S932A | | | | |
| 679/680 | L24W/L28S/L29T/P39Q/Q50V/ | H711C | + | + | |
| | A62L/P78E/D87E/S135Q/T150S/ | | | | |
| | T266N/R267K/A437G/T486E/E522V/ | | | | |
| | L569T/L670T/T692G/A711C/ | | | | |
| | L736M/A750P/A812E/Q830K/ | | | | |
| | G842S/L871E/R883H/Q894G/ | | | | |
| | V913R/S932A | | | | |
| 681/682 | L24W/L28S/L29T/P39Q/Q50W/ | V50W | + | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 683/684 | L24W/L28S/L29T/P39Q/Q50V/ | V522C | + | + | |
| | A62L/P78E/D87E/S135Q/T150S/ | | | | |
| | T266N/R267K/A437G/T486E/E522C/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 685/686 | L24W/L28S/L29T/P39Q/Q50V/ | E486T | + | ++ | + |
| | A62L/P78E/D87E/S135Q/T150S/ | | | | |
| | T266N/R267K/A437G/E522V/L569T/ | | | | |
| | L670T/T692G/A711H/L736M/ | | | | |
| | A750P/A812E/Q830K/G842S/ | | | | |
| | L871E/R883H/Q894G/V913R/S932A | | | | |
| 687/688 | L24W/L28S/L29T/P39Q/Q50V/ | A932S | ++ | ++ | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/V913R | | | | |
| 689/690 | L24W/L28S/L29W/P39Q/Q50V/ | T29W | ++ | | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 691/692 | L24W/L28S/L29T/P39Q/Q50V/ | E812G | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812G/Q830K/ | | | | |
| | G842S/L871E/R883H/Q894G/ | | | | |
| | V913R/S932A | | | | |
| 693/694 | L24W/L28S/L29T/P39Q/Q50V/ | E78D | + | ++ | +++ |
| | A62L/P78D/D87E/S135Q/T150S/ | | | | |
| | T266N/R267K/A437G/T486E/ | | | | |
| | E522V/L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 695/696 | L24W/L28S/L29T/P39Q/Q50V/ | E871D | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871D/R883H/Q894G/ | | | | |
| | V913R/S932A | | | | |
| 697/698 | L24W/L28S/L29T/P39Q/Q50V/ | G692I | | + | + |
| | A62L/P78E/D87E/S135Q/T150S/ | | | | |
| | T266N/R267K/A437G/T486E/E522V/ | | | | |
| | L569T/L670T/T692I/A711H/ | | | | |
| | L736M/A750P/A812E/Q830K/ | | | | |
| | G842S/L871E/R883H/Q894G/ | | | | |
| | V913R/S932A | | | | |
| 699/700 | L24W/L28S/L29F/P39Q/Q50V/A62L/ | T29F | + | | ++ |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 701/702 | L24W/L28S/L29T/P39Q/Q50V/ | V522M | + | + | |
| | A62L/P78E/D87E/S135Q/T150S/ | | | | |
| | T266N/R267K/A437G/T486E/E522M/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 703/704 | L24W/L28S/L29T/P39Q/Q50V/ | T670N | + | + | + |
| | A62L/P78E/D87E/S135Q/T150S/ | | | | |
| | T266N/R267K/A437G/T486E/E522V/ | | | | |
| | L569T/L670N/T692G/A711H/ | | | | |
| | L736M/A750P/A812E/Q830K/ | | | | |
| | G842S/L871E/R883H/Q894G/ | | | | |
| | V913R/S932A | | | | |
| 705/706 | L24W/L28S/L29M/P39Q/Q50V/ | T29M | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 707/708 | L24W/L28S/L29T/P39Q/Q50V/ | E486G | + | + | + |
| | A62L/P78E/D87E/S135Q/T150S/ | | | | |
| | T266N/R267K/A437G/T486G/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 709/710 | L24W/L28S/L29T/P39Q/Q50V/ | A932K | | | +++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932K | | | | |
| 711/712 | L24W/L28S/L29T/P39Q/Q50V/ | L62Q | | + | |
| | A62Q/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 713/714 | L24W/L28S/L29T/P39Q/Q50V/ | K830S | | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830S/ | | | | |
| | G842S/L871E/R883H/Q894G/ | | | | |
| | V913R/S932A | | | | |
| 715/716 | L24W/L28S/L29T/P39Q/Q50V/ | E812S | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812S/Q830K/ | | | | |
| | G842S/L871E/R883H/Q894G/ | | | | |
| | V913R/S932A | | | | |
| 717/718 | L24W/L28S/L29T/P39Q/Q50V/ | K830G | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830G/ | | | | |
| | G842S/L871E/R883H/Q894G/ | | | | |
| | V913R/S932A | | | | |
| 719/720 | L24W/L28S/L29T/P39Q/Q50V/ | E486M | + | ++ | +++ |
| | A62L/P78E/D87E/S135Q/T150S/ | | | | |
| | T266N/R267K/A437G/T486M/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 721/722 | L24W/L28S/L29T/P39Q/Q50V/ | H711K | + | ++ | |
| | A62L/P78E/D87E/S135Q/T150S/ | | | | |
| | T266N/R267K/A437G/T486E/E522V/ | | | | |
| | L569T/L670T/T692G/A711K/ | | | | |
| | L736M/A750P/A812E/Q830K/ | | | | |
| | G842S/L871E/R883H/Q894G/ | | | | |
| | V913R/S932A | | | | |
| 723/724 | L24W/L28S/L29T/P39Q/Q50V/ | A932H | ++ | ++ | +++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932H | | | | |
| 725/726 | L24W/L28S/L29P/P39Q/Q50V/A62L/ | T29P | ++ | + | |
| | P78E/D87E/S135Q/T150S/T266N/ | | | | |
| | R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 727/728 | L24W/L28S/L29T/P39Q/Q50V/ | G692W | + | + | |
| | A62L/P78E/D87E/S135Q/T150S/ | | | | |
| | T266N/R267K/A437G/T486E/E522V/ | | | | |
| | L569T/L670T/T692W/A711H/ | | | | |
| | L736M/A750P/A812E/Q830K/ | | | | |
| | G842S/L871E/R883H/Q894G/ | | | | |
| | V913R/S932A | | | | |
| 729/730 | L24W/L28S/L29Y/P39Q/Q50V/ | T29Y | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 731/732 | L24W/L28S/L29T/P39Q/Q50V/ | G692C | + | + | + |
| | A62L/P78E/D87E/S135Q/T150S/ | | | | |
| | T266N/R267K/A437G/T486E/E522V/ | | | | |
| | L569T/L670T/T692C/A711H/ | | | | |
| | L736M/A750P/A812E/Q830K/ | | | | |
| | G842S/L871E/R883H/Q894G/ | | | | |
| | V913R/S932A | | | | |
| 733/734 | L24W/L28S/L29G/P39Q/Q50V/ | T29G | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932A | | | | |
| 735/736 | L24W/L28S/L29T/P39Q/Q50V/ | E812D | | + | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812D/Q830K/ | | | | |
| | G842S/L871E/R883H/Q894G/ | | | | |
| | V913R/S932A | | | | |
| 737/738 | L24W/L28S/L29T/P39Q/Q50V/ | A932C | ++ | + | +++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913R/S932C | | | | |
| 739/740 | L24W/L28S/L29T/P39Q/Q50V/ | E871I | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871I/R883H/Q894G/ | | | | |
| | V913R/S932A | | | | |
| 741/742 | L24W/L28S/L29T/P39Q/Q50V/ | G692Y | + | + | |
| | A62L/P78E/D87E/S135Q/T150S/ | | | | |
| | T266N/R267K/A437G/T486E/E522V/ | | | | |
| | L569T/L670T/T692Y/A711H/ | | | | |
| | L736M/A750P/A812E/Q830K/ | | | | |
| | G842S/L871E/R883H/Q894G/ | | | | |
| | V913R/S932A | | | | |
| 743/744 | L24W/L28S/L29T/P39Q/Q50V/ | P750Q | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750Q/A812E/Q830K/ | | | | |
| | G842S/L871E/R883H/Q894G/ | | | | |
| | V913R/S932A | | | | |
| 745/746 | L24W/L28S/L29T/P39Q/Q50V/ | K830H | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830H/ | | | | |
| | G842S/L871E/R883H/Q894G/ | | | | |
| | V913R/S932A | | | | |
| 747/748 | L24W/L28S/L29T/P39Q/Q50V/ | S842C | + | | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842C/L871E/R883H/Q894G/ | | | | |
| | V913R/S932A | | | | |
| 749/750 | L24W/L28S/L29T/P39Q/Q50V/ | E871S | + | ++ | + |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871S/R883H/Q894G/ | | | | |
| | V913R/S932A | | | | |
| 751/752 | L24W/L28S/L29T/P39Q/Q50V/ | E871C | + | + | ++ |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871C/R883H/Q894G/ | | | | |
| | V913R/S932A | | | | |
| 753/754 | L24W/L28S/L29T/P39Q/Q50V/ | R913W | + | + | |
| | A62L/P78E/D87E/S135Q/T150S/ | | | | |
| | T266N/R267K/A437G/T486E/E522V/ | | | | |
| | L569T/L670T/T692G/A711H/ | | | | |
| | L736M/A750P/A812E/Q830K/ | | | | |
| | G842S/L871E/R883H/Q894G/ | | | | |
| | V913W/S932A | | | | |

¹All activities were determined relative to the reference polypeptide of SEQ ID NO: 12. Levels of increased activity are defined as follows: “+” 0.9-1.1, “++” >1.1, “+++” >1.5, “++++” >2.5

### Example 4

### Production of GAA Variants

### Production of GAA in EXPI293F™ Cells

Milligram-scale production of GAA variants was achieved by transient transfection of EXPI293F™ cells (ThermoFisher Scientific) using the lipofection method with EXPIFECTAMINE™ 293 Reagent (ThermoFisher Scientific) in EXPI293™ Expression Medium (ThermoFisher Scientific). GAA variants fused to an N-terminal synthetic mammalian signal peptide were subcloned into the mammalian expression vectors pDH or pcDNA 3.1(+) as described in Example 1. EXPI293F™ cells were transfected with plasmid DNA and grown in suspension for 4-7 days. Conditioned media was then harvested, clarified by centrifugation and filtration or diatomaceous earth and stored at −80° C. until purification.

### Example 5

### Purification of GAA Variants

GAA variants (SEQ ID NO: 2, 4, 12, 8, 6, 14, 10) produced in EXPI293F™ cells as described in Example 4, were purified from mammalian culture supernatant as described in the literature (Yasuda et al., Prot. Exp. Pur., 2004, 37:499-506). Concanavalin A resin (Sigma Aldrich) was equilibrated with 0.1M sodium acetate, 0.1M NaCl, 1 mM MgCl₂, CaCl₂, and MnCl₂, pH 6.0 (Concanavalin A binding buffer). Supernatant was concentrated 10-fold and diluted 2-fold into equilibration buffer (0.1M sodium acetate, 0.1M NaCl, 1 mM MgCl₂, CaCl₂, and MnCl₂, pH 6.0) before it was loaded onto the column. After loading, the column was washed with 10 column volumes of Concanavalin A binding buffer and the bound protein was eluted with Concanavalin A binding buffer supplemented with 0.9M methyl-α-D-mannopyranoside and 0.9M methyl-α-D-glucopyranoside. Eluted protein was concentrated, and the buffer exchanged into storage buffer (20 mM sodium phosphate, 150 mM sodium chloride, 185 μM TWEEN®-20 non-ionic detergent, pH 6.0) using an AMICON® Ultra 15 mL filtration unit with a 50 kDa molecular weight cut off membrane (Millipore). The GAA in storage buffer was sterile filtered through ANOTOP® 0.2 μm syringe filters (Whatman), and stored at −80° C. The purification process produced 60-100 mg of purified protein/L of culture supernatant.

### Example 6

### In Vitro Characterization of GAA Variants

In this Example, experiments conducted to characterize GAA variants produced as indicated herein are described.

Stability at Neutral pH of rhGAA and GAA Variants

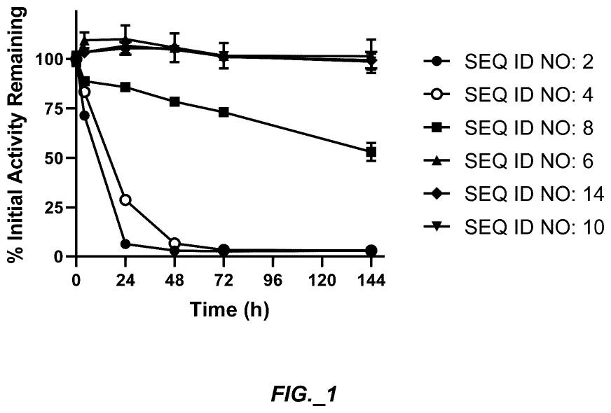

GAA variant stability to neutral pH was determined by incubating purified variants at 214 nM in MEM complete growth medium (pH 7.4) in a 96-well plate. The plates were incubated at 37° C. for up to 144 hours. At each timepoint, 10 μL of neutral-pH-challenged sample was transferred into a BioRad hardshell plate and frozen immediately at −80° C. At the conclusion of experiment all plates were processed simultaneously via addition of 50 μL of 1.5 mM 4-MU-GLU in McIlvaine Buffer for 30 minutes, with agitation at 400 rpm, at 37° C. Reactions were quenched with 100 μL of Na₂CO₃ (0.5M, pH 10.5), 100 μL was transferred into a black 96 well plate and hydrolysis was assessed by quantifying the released fluorescent methylumbelliferone, using an Envision microplate reader (ex 355/em 460 nm). The results from this assay are presented in FIG. 1.

Melting Temperature of rhGAA and GAA Variants at Neutral and Lysosomal pH

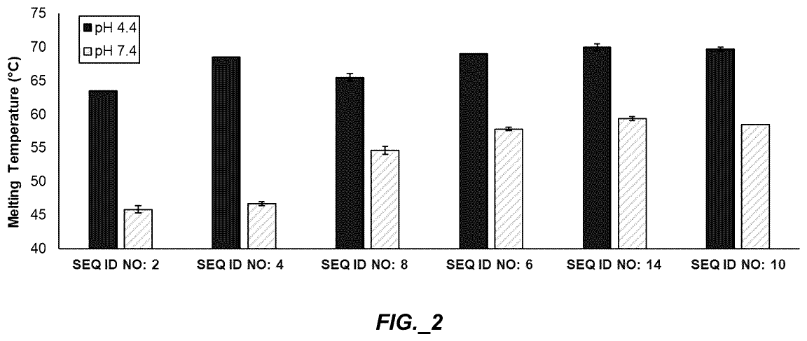

The melting temperature of rhGAA and GAA variants was determined by differential scanning fluorimetry at neutral and lysosomal pH. GAA enzyme variants were diluted to 1 mg/mL in DPBS (pH 6.2). 40 μL of pH 4.4 or pH 7.4 McIlvaine Buffer containing 1×SYPRO Orange was mixed with 10 μL of each enzyme solution (n=3) in separate wells of 96-well skirted BioRad plates. The plates were sealed with optically clear film and run on a CFX connect real-time PCR system using the manufacturer's recommended method from 25-95° C. Data were analyzed by BioRad Software. The results of these assays are presented in FIG. 2.

Stability in Plasma of rhGAA and GAA Variants

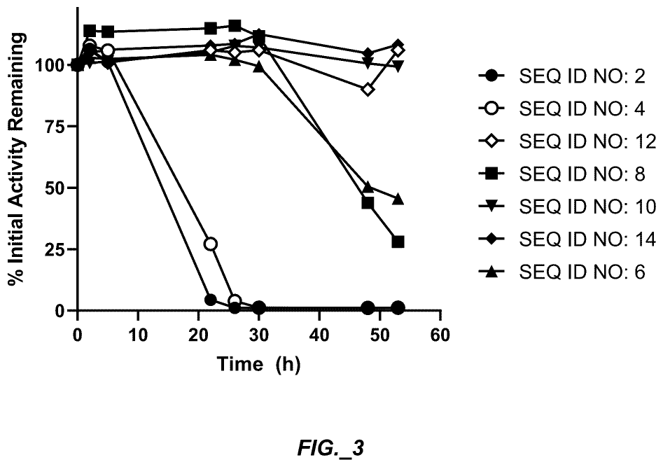

GAA variant stability to cynomolgus plasma was assessed over a time course 75 hours. Purified GAA variants were diluted into GAA storage buffer (20 mM Sodium Phosphate, 150 mM NaCl, 185 μM polysorbate 20, pH 6.0) to 100 μg/mL (2×assay concentration) and dispensed in triplicate (100 μL/well) into a BioRad hardshell plate. 100 μL of cynomolgus plasma was added and mixed thoroughly. The plasma challenge plate was sealed and incubated for up to 75 hours with agitation (400 rpm) at 37° C. At timepoints across the challenge time, the residual activity remaining of GAA variants was measured by transferring 10 μL of the plasma-challenged solution into 50 μL of 1.5 mM 4-MU-GLU in McIlvaine Buffer, pH 4.4, in black 96-well plates for 30 minutes with agitation (400 rpm) at 37° C. Reactions were quenched with 100 μL of Na₂CO₃ (0.5M, pH 10.5), and hydrolysis was assessed by quantifying the released fluorescent methylumbelliferone using an Envision microplate reader (ex 355/em 460 nm). The results from this assay are presented in FIG. 3.

### Cellular Uptake in Pompe Fibroblasts or C2C12 GAA Knockout Myoblasts of Purified GAA Variants

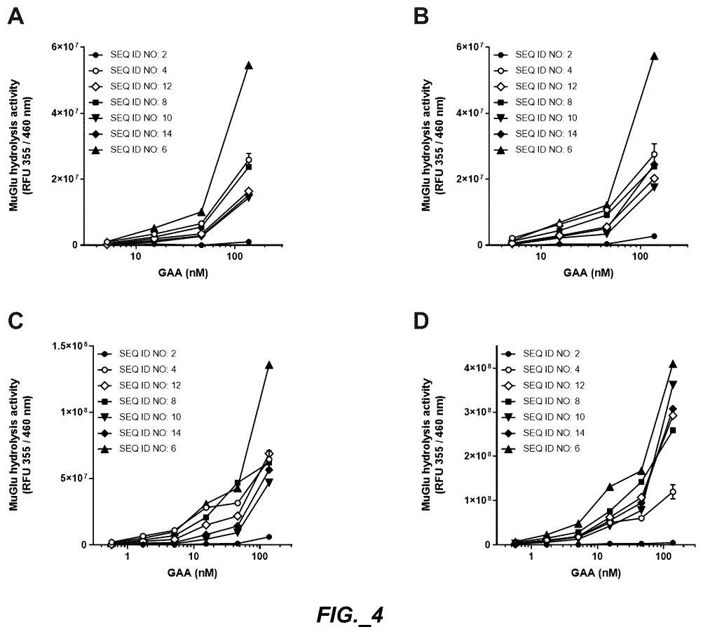

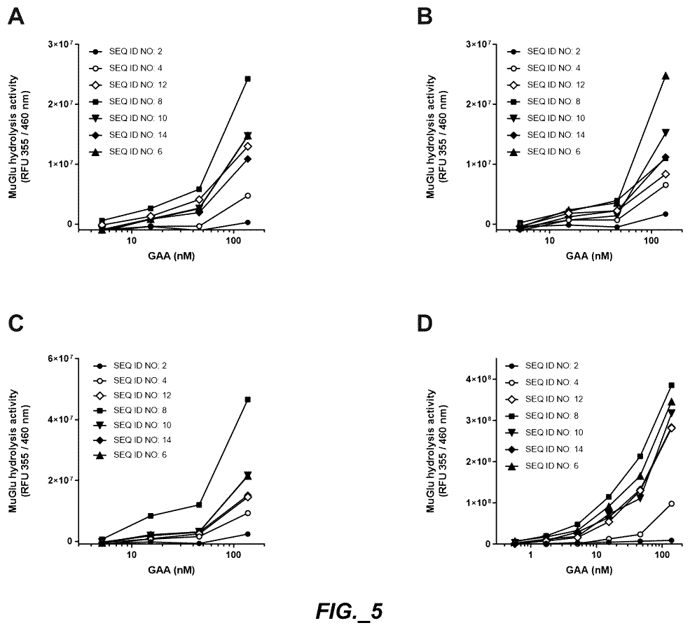

The capability of GAA variants for cross-correction of cells as compared to reference enzymes (SEQ ID NOS: 2 and 4) was determined. Pompe fibroblasts (GM00248, Coriell Institute for Medical Research) or C2C12 GAA knockout myoblasts were seeded into a black walled, clear bottom 96-well plate (Costar, #3604) in standard complete growth medium and allowed to reach confluency (2-3 days at 37° C., 5% CO₂). After reaching confluency, standard complete growth medium was removed using an automated BioMek i5 liquid handler. Enzymes purified as described in Example 5, were added to cells in a dilution series of concentrations ranging from 0-214 nM GAA/mL in standard complete growth media and allowed to incubate for 1, 4, 24 or 96 hours at 37° C., 5% CO₂. At the conclusion of treatment, media containing GAA variants were aspirated using an automated BioMek i5 liquid handler. The cells were briefly washed with 150 μL 1×DPBS/well, and the DPBS was removed by an automated BioMek i5 liquid handler. Then, 200 μL standard complete growth medium was added to each well, and the plates were returned to the incubator for the remainder of the 96 hour experiment (95 to 0 hr, depending on treatment time). At the conclusion of experiment, MEM complete growth media was removed using an automated BioMek i5 liquid handler. The cells were washed with 150 μL 1×DPBS/well, and the DPBS was removed using an automated BioMek i5 liquid handler. Cells were lysed via addition of 50 μL of McIlvaine buffer, pH 4.4, supplemented with 0.5% TRITON X-100™ non-ionic surfactant (Sigma #93443) and agitation at room temperature for 30 minutes. GAA activity was assessed by addition of 50 μL of 1.5 mM 4-MU-GLU in McIlvaine buffer, pH 4.4. The plates were sealed, incubated at 37° C. for 300-360 minutes with agitation at 400 rpm, prior to quenching with 100 μL of 0.5M sodium carbonate pH 10.5. Hydrolysis was analyzed using an EnVision (Perkin Elmer) microplate reader monitoring fluorescence (Ex. 355 nm, Em. 460 nm). FIG. 4, Panels A, B, C, and D provide graphs showing the activity in Pompe fibroblast lysates following treatment with purified GAA variants for durations of 1 to 96 hours. FIG. 5, Panels A, B, C, and D provide graphs showing the activity in C2C12 GAA KO myoblast lysates of purified GAA variants for durations of 1 to 96 hours.

### Example 7

### In Vitro Characterization of GAA Variant Expression in Myoblasts

In this Example, experiments conducted to characterize expression and activity of GAA variants in myoblasts as indicated herein are described.

rhGAA and GAA Variant 4-MU-GLU and Glycogen Hydrolysis Activity in Transiently Transfected Myoblasts

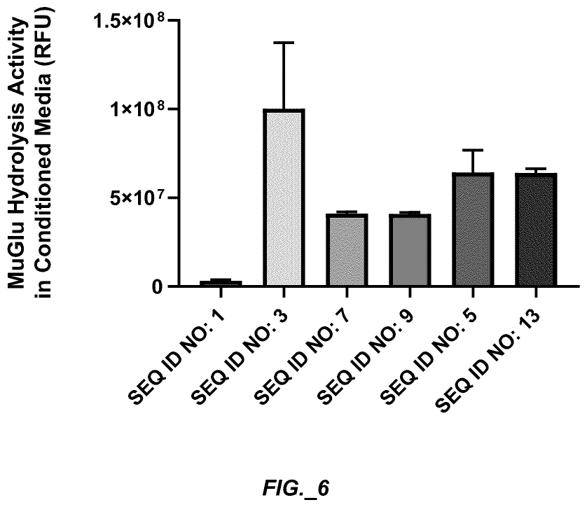

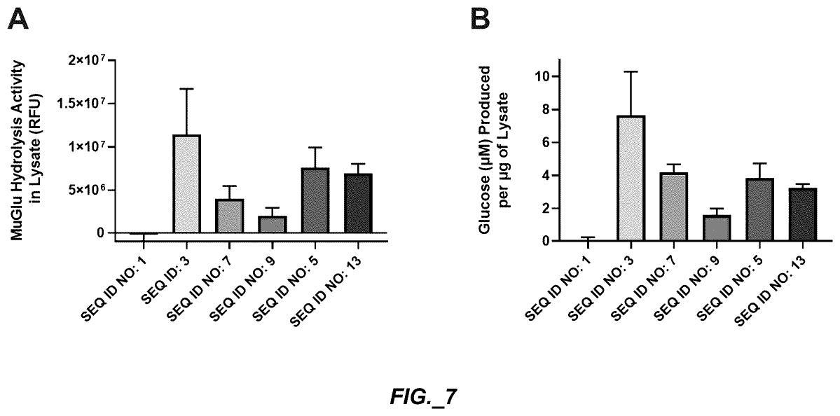

The expression efficiency and activity of wild type GAA and GAA variants was assessed in myoblasts using transient transfection. C2C12 GAA knockout myoblasts were seeded into 12-well plates in complete growth medium (Dulbecco's Modified Eagle's Medium with 10% fetal bovine serum) and allowed to adhere for 24 hours to a confluency of ˜50%. Cells were transfected with plasmid DNA of GAA variants using jetOPTIMUS® transfection polyplus. Plates were returned to the incubator for 4 hours before a media exchange. At 3 days post-transfection the conditioned media was assessed for GAA activity and myoblasts were harvested to assess GAA activity in lysates. GAA activity in conditioned media was determined against 4-MU-GLU, and was assessed via incubation of 20 μL of conditioned media with 50 μL of 1.5 mM 4-MU-GLU in McIlvaine Buffer, pH 4.4, in black 96-well plates for 4 hours with agitation (400 rpm) at 37° C. Hydrolysis was assessed by quantifying the released fluorescent methylumbelliferone, using an Envision microplate reader (ex 355/em 460 nm). To generate lysates, myoblasts were harvested with trypsin and centrifugation. Cell pellets were washed with DPBS and lysed in 52 μL GAA lysis buffer (0.2M sodium acetate, 0.4M potassium chloride, 0.5% Triton X-100, pH 4.3) on ice with intermittent vortexing for 30 minutes. Lysates were clarified (10 minutes at 20,000 RCF) and protein concentration was determined by BCA and normalized. GAA activity in lysates against 4-MU-GLU was assessed via incubation of 2 or 4 μL of normalized lysate with 50 μL of 1.5 mM 4-MU-GLU in McIlvaine Buffer, pH 4.4, in black 96-well plates for 4 hours with agitation (400 rpm) at 37° C. Hydrolysis was assessed by quantifying the released fluorescent methylumbelliferone, using an Envision microplate reader (ex 355/em 460 nm). Glycogen hydrolysis activity of lysates was determined by incubation of lysates with glycogen (100 mg/ml prepared in GAA reaction buffer of 0.1M sodium acetate, 0.1M NaCl, 0.5 mg/mL BSA at pH 4.3) for 1 hour at 37° C., 400 rpm. Glycogen hydrolysis reactions were quenched/neutralized via addition of 90 μL of stop buffer (133 mM glycine, 83 mM sodium carbonate, pH 10.7). Quenched hydrolysis reactions were diluted 1:20 in Amplex red reaction buffer (Invitrogen Amplex Red Glucose/Glucose Oxidase Assay Kit #A22189). 50 μL of quenched hydrolysis reaction dilutions and 50 μL of glucose standards (at 0, 3.2, 6.25, 12.5, 25, 50, and 100 μM) were added to a black 96-well plate. 50 μL of the Amplex Reagent Mix (Amplex Red/HRP/Glucose oxidase) was added to all wells and the plate was incubated at room temperature with gentle shaking, protected from light for 30 minutes. Red fluorescent resorufin (formed from the reaction of Amplex Red with hydrogen peroxide produced from the glucose oxidase—HRP coupled reactions) was quantified with a Spectramax EM microplate reader (ex 540/em 590 nm). FIG. 6 provides a graph showing the activity in conditioned media of C2C12 GAA KO myoblasts following transient transfections. FIG. 7 provides graphs showing, Panels A and B, the activity in lysates of C2C12 GAA KO myoblasts following transient transfections.

### Example 8

Identification of Active GAA Variants with Reduced Immunogenicity

In this Example, experiments conducted to characterize expression and activity of GAA variants in myoblasts as indicated herein are described.

Putative T-cell epitopes in a WT GAA of SEQ ID NO: 2 or the engineered GAA variant SEQ ID NO: 12 were identified using the Immune Epitope Database (IEDB; Immune Epitope Database and Analysis Resource website) tools, as known in the art and proprietary statistical analysis tools (See e.g., iedb.org, and Vita et al., Nucl. Acids Res., 2020, 38 (Database issue): D854-62. Epub 2009 Nov. 11). The WT GAA or the engineered GAA variant was parsed into all possible 15-mer analysis frames, with each frame overlapping the last by 14 amino acids. The 15-mer analysis frames were evaluated for immunogenic potential by scoring their predicted binding to eight common class II HLA-DR alleles (DRB1*0101, DRB1*0301, DRB1*0401, DRB1*0701, DRB1*0801, DRB1*1101, DRB1*1301, and DRB1*1501) that collectively cover 77% of the world population (See e.g., iedb.org and Bui et al., 2006, BMC Bioinformatics, 7:153), using methods recommended on the IEDB website. Potential T-cell epitope clusters contained within the enzyme (i.e., sub-regions contained within GAA which have an unusually high potential for immunogenicity) were identified using statistical analysis tools, as known in the art.

GAA variants that were identified in Example 3 to be active in assays described in Example 2 were analyzed for their levels of predicted immunogenicity by evaluating their binding to the eight common Class II HLA-DR alleles. The total immunogenicity score and immunogenic hit count were calculated for each variant. The total immunogenicity score (TIS) reflects the total number of predicted binding events between the eight MHC Class II alleles (described above) and peptide 15-mers across the sequence (i.e., a higher score indicates a higher level of predicted immunogenicity). The immunogenic “hit count” (IHC) indicates the number of peptide 15-mers that were predicted to bind to 4 or more of the 8 common alleles; these regions of the sequence have a particularly high potential for immunogenicity across a population (i.e., a higher score indicates a higher potential for immunogenicity). Mutations resulting in a reduced total immunogenicity score and/or an immunogenic hit count compared to the reference sequence were considered to be potential “deimmunizing mutations” and are shown in Table 4-1.

TABLE 4-1
Reduction of Total Immunogenicity Score
(TIS) and Immunogenic Hit Count (IHC) for GAA
Variants Relative to SEQ ID NO: 2 and SEQ ID NO: 12

| | TIC | IHC | TIC | IHC |
|---|---|---|---|---|
| SEQ | reduction | reduction | reduction | reduction |
| ID | relative | relative | relative | relative |
| NO: | to SEQ ID | to SEQ ID | to SEQ ID | to SEQ ID |
| (nt/aa) | NO: 2 | NO: 2 | NO: 12 | NO: 12 |
| 1/2 | | | | |
| 3/4 | + | + | | |
| 5/6 | ++ | +++ | | |
| 7/8 | + | ++ | | |
| 9/10 | + | ++ | | |
| 11/12 | ++ | +++ | | |
| 13/14 | ++ | +++ | ++++ | + |
| 15/16 | ++ | +++ | | |
| 17/18 | ++ | +++ | | |
| 19/20 | ++ | +++ | ++ | |
| 21/22 | ++ | +++ | | |
| 23/24 | ++ | ++ | | |
| 25/26 | ++ | +++ | + | |
| 27/28 | ++ | +++ | + | |
| 29/30 | ++ | +++ | | |
| 31/32 | ++ | ++ | | |
| 33/34 | ++ | +++ | + | |
| 35/36 | ++ | +++ | | |
| 37/38 | ++ | +++ | | |
| 39/40 | ++ | +++ | ++ | |
| 41/42 | ++ | +++ | + | |
| 43/44 | ++ | +++ | | |
| 45/46 | ++ | +++ | | |
| 47/48 | ++ | +++ | | |
| 49/50 | ++ | +++ | | |
| 51/52 | ++ | +++ | | |
| 53/54 | ++ | +++ | | |
| 55/56 | ++ | ++ | | |
| 57/58 | ++ | +++ | | |
| 59/60 | ++ | +++ | | |
| 61/62 | ++ | +++ | | |
| 63/64 | ++ | +++ | | |
| 65/66 | ++ | +++ | + | |
| 67/68 | ++ | ++ | | |
| 69/70 | ++ | +++ | | |
| 71/72 | ++ | +++ | + | |
| 73/74 | ++ | +++ | | |
| 75/76 | ++ | +++ | | |
| 77/78 | ++ | +++ | | |
| 79/80 | ++ | +++ | +++ | |
| 81/82 | ++ | +++ | | |
| 83/84 | ++ | +++ | | |
| 85/86 | ++ | +++ | | |
| 87/88 | ++ | +++ | | |
| 89/90 | ++ | +++ | ++ | |
| 91/92 | ++ | +++ | | |
| 93/94 | ++ | +++ | | |
| 95/96 | ++ | +++ | + | |
| 97/98 | ++ | +++ | | |
| 99/100 | ++ | +++ | ++ | |
| 101/102 | ++ | +++ | + | |
| 103/104 | ++ | +++ | | |
| 105/106 | ++ | +++ | | |
| 107/108 | ++ | +++ | | |
| 109/110 | ++ | +++ | | |
| 111/112 | ++ | +++ | | |
| 113/114 | ++ | +++ | | |
| 115/116 | ++ | +++ | | |
| 117/118 | ++ | +++ | | |
| 119/120 | + | ++ | | |
| 121/122 | ++ | +++ | | |
| 123/124 | ++ | +++ | | |
| 125/126 | ++ | +++ | + | |
| 127/128 | ++ | +++ | | |
| 129/130 | ++ | +++ | | |
| 131/132 | ++ | +++ | | |
| 133/134 | ++ | +++ | | |
| 135/136 | ++ | +++ | | |
| 137/138 | ++ | +++ | | |
| 139/140 | ++ | +++ | | |
| 141/142 | ++ | +++ | + | |
| 143/144 | ++ | +++ | | |
| 145/146 | ++ | +++ | | |
| 147/148 | ++ | +++ | | |
| 149/150 | ++ | ++ | | |
| 151/152 | ++ | +++ | + | |
| 153/154 | ++ | +++ | | |
| 155/156 | ++ | +++ | | |
| 157/158 | ++ | +++ | | |
| 159/160 | ++ | +++ | | |
| 161/162 | ++ | +++ | | |
| 163/164 | ++ | +++ | | |
| 165/166 | ++ | +++ | | |
| 167/168 | ++ | +++ | + | |
| 169/170 | ++ | +++ | + | |
| 171/172 | ++ | +++ | | |
| 173/174 | + | ++ | | |
| 175/176 | ++ | +++ | | |
| 177/178 | ++ | +++ | | |
| 179/180 | ++ | ++ | | |
| 181/182 | ++ | +++ | + | |
| 183/184 | ++ | +++ | | |
| 185/186 | ++ | +++ | | |
| 187/188 | ++ | +++ | | |
| 189/190 | ++ | +++ | | |
| 191/192 | ++ | +++ | | |
| 193/194 | ++ | +++ | | |
| 195/196 | ++ | +++ | | |
| 197/198 | ++ | +++ | | |
| 199/200 | ++ | +++ | | |
| 201/202 | ++ | +++ | | |
| 203/204 | ++ | +++ | | |
| 205/206 | ++ | +++ | | |
| 207/208 | ++ | +++ | | |
| 209/210 | ++ | +++ | | |
| 211/212 | ++ | +++ | | |
| 213/214 | ++ | +++ | | |
| 215/216 | ++ | +++ | | |
| 217/218 | ++ | +++ | | |
| 219/220 | ++ | +++ | | |
| 221/222 | ++ | +++ | | |
| 223/224 | ++ | +++ | | |
| 225/226 | ++ | +++ | +++ | |
| 227/228 | ++ | +++ | | |
| 229/230 | ++ | +++ | | |
| 231/232 | ++ | +++ | | |
| 233/234 | ++ | +++ | + | |
| 235/236 | ++ | +++ | | |
| 237/238 | ++ | +++ | | |
| 239/240 | ++ | +++ | | |
| 241/242 | ++ | +++ | | |
| 243/244 | ++ | +++ | | |
| 245/246 | ++ | +++ | ++ | |
| 247/248 | ++ | +++ | | |
| 249/250 | ++ | +++ | + | |
| 251/252 | ++ | +++ | + | |
| 253/254 | ++ | +++ | | |
| 255/256 | ++ | +++ | ++ | |
| 257/258 | ++ | +++ | | |
| 259/260 | ++ | +++ | | |
| 261/262 | ++ | +++ | | |
| 263/264 | ++ | +++ | | |
| 265/266 | ++ | +++ | + | |
| 267/268 | ++ | +++ | | |
| 269/270 | ++ | +++ | + | |
| 271/272 | ++ | +++ | | |
| 273/274 | ++ | +++ | + | |
| 275/276 | ++ | +++ | | |
| 277/278 | ++ | +++ | | |
| 279/280 | ++ | +++ | | |
| 281/282 | ++ | +++ | | |
| 283/284 | ++ | ++ | | |
| 285/286 | ++ | +++ | | |
| 287/288 | ++ | +++ | + | |
| 289/290 | ++ | +++ | | |
| 291/292 | ++ | +++ | | |
| 293/294 | ++ | +++ | | |
| 295/296 | ++ | +++ | | |
| 297/298 | ++ | +++ | | |
| 299/300 | ++ | +++ | | |
| 301/302 | ++ | +++ | | |
| 303/304 | ++ | +++ | +++ | |
| 305/306 | ++ | +++ | | |
| 307/308 | ++ | +++ | | |
| 309/310 | ++ | +++ | +++ | |
| 311/312 | ++ | +++ | | |
| 313/314 | ++ | +++ | | |
| 315/316 | ++ | +++ | + | |
| 317/318 | ++ | +++ | | |
| 319/320 | ++ | +++ | | |
| 321/322 | ++ | +++ | | |
| 323/324 | ++ | +++ | ++ | |
| 325/326 | ++ | +++ | | |
| 327/328 | ++ | +++ | | |
| 329/330 | ++ | +++ | +++ | + |
| 331/332 | ++ | +++ | | |
| 333/334 | ++ | ++ | | |
| 335/336 | ++ | +++ | | |
| 337/338 | ++ | +++ | | |
| 339/340 | ++ | +++ | | |
| 341/342 | ++ | +++ | | |
| 343/344 | ++ | +++ | +++ | |
| 345/346 | ++ | +++ | | |
| 347/348 | ++ | +++ | | |
| 349/350 | ++ | ++ | | |
| 351/352 | ++ | +++ | + | |
| 353/354 | ++ | +++ | | |
| 355/356 | ++ | +++ | | |
| 357/358 | ++ | +++ | | |
| 359/360 | ++ | +++ | | |
| 361/362 | ++ | +++ | | |
| 363/364 | ++ | +++ | | |
| 365/366 | ++ | +++ | | |
| 367/368 | ++ | +++ | | |
| 369/370 | ++ | +++ | | |
| 371/372 | ++ | ++ | | |
| 373/374 | ++ | +++ | | |
| 375/376 | ++ | +++ | ++++ | +++ |
| 377/378 | ++ | +++ | +++ | |
| 379/380 | ++ | +++ | + | |
| 381/382 | ++ | +++ | | |
| 383/384 | ++ | +++ | | |
| 385/386 | ++ | +++ | | |
| 387/388 | ++ | +++ | | |
| 389/390 | ++ | +++ | | |
| 391/392 | ++ | +++ | + | |
| 393/394 | ++ | +++ | + | |
| 395/396 | ++ | +++ | | |
| 397/398 | ++ | +++ | + | |
| 399/400 | ++ | +++ | | |
| 401/402 | ++ | +++ | | |
| 403/404 | ++ | +++ | | |
| 405/406 | + | ++ | | |
| 407/408 | ++ | +++ | | |
| 409/410 | ++ | +++ | | |
| 411/412 | ++ | +++ | + | |
| 413/414 | ++ | +++ | | |
| 415/416 | ++ | +++ | | |
| 417/418 | ++ | +++ | | |
| 419/420 | ++ | +++ | ++ | |
| 421/422 | ++ | +++ | | |
| 423/424 | ++ | +++ | | |
| 425/426 | ++ | +++ | | |
| 427/428 | ++ | +++ | | |
| 429/430 | ++ | +++ | | |
| 431/432 | ++ | +++ | + | |
| 433/434 | ++ | +++ | +++ | + |
| 435/436 | ++ | +++ | | |
| 437/438 | ++ | +++ | | |
| 439/440 | ++ | +++ | | |
| 441/442 | ++ | +++ | | |
| 443/444 | ++ | +++ | | |
| 445/446 | ++ | +++ | + | |
| 447/448 | ++ | +++ | + | |
| 449/450 | ++ | +++ | ++++ | + |
| 451/452 | ++ | +++ | | |
| 453/454 | ++ | +++ | | |
| 455/456 | ++ | +++ | ++ | |
| 457/458 | ++ | +++ | | |
| 459/460 | ++ | +++ | | |
| 461/462 | ++ | +++ | + | |
| 463/464 | ++ | +++ | | |
| 465/466 | ++ | +++ | + | |
| 467/468 | ++ | +++ | | |
| 469/470 | ++ | +++ | | |
| 471/472 | ++ | +++ | | |
| 473/474 | + | ++ | | |
| 475/476 | ++ | +++ | | |
| 477/478 | ++ | +++ | + | |
| 479/480 | ++ | +++ | | |
| 481/482 | ++ | +++ | + | |
| 483/484 | ++ | +++ | | |
| 485/486 | ++ | +++ | | |
| 487/488 | ++ | +++ | + | |
| 489/490 | ++ | +++ | | |
| 491/492 | ++ | +++ | | |
| 493/494 | ++ | +++ | + | |
| 495/496 | ++ | +++ | | |
| 497/498 | ++ | +++ | | |
| 499/500 | ++ | +++ | | |
| 501/502 | ++ | +++ | | |
| 503/504 | ++ | +++ | + | |
| 505/506 | ++ | +++ | | |
| 507/508 | ++ | +++ | | |
| 509/510 | ++ | +++ | | |
| 511/512 | ++ | +++ | | |
| 513/514 | ++ | +++ | | |
| 515/516 | ++ | +++ | | |
| 517/518 | ++ | +++ | + | |
| 519/520 | ++ | +++ | | |
| 521/522 | ++ | +++ | | |
| 523/524 | ++ | ++ | | |
| 525/526 | ++ | +++ | + | |
| 527/528 | ++ | +++ | ++ | |
| 529/530 | ++ | +++ | | |
| 531/532 | ++ | +++ | + | |
| 533/534 | ++ | +++ | + | |
| 535/536 | ++ | +++ | + | |
| 537/538 | ++ | +++ | +++ | |
| 539/540 | + | ++ | | |
| 541/542 | ++ | +++ | | |
| 543/544 | ++ | +++ | + | |
| 545/546 | ++ | +++ | | |
| 547/548 | ++ | +++ | | |
| 549/550 | ++ | +++ | | |
| 551/552 | ++ | +++ | | |
| 553/554 | ++ | +++ | + | |
| 555/556 | ++ | +++ | | |
| 557/558 | ++ | +++ | ++ | |
| 559/560 | ++ | +++ | ++++ | + |
| 561/562 | ++ | +++ | | |
| 563/564 | ++ | +++ | | |
| 565/566 | ++ | +++ | | |
| 567/568 | ++ | ++ | | |
| 569/570 | ++ | +++ | | |
| 571/572 | ++ | +++ | | |
| 573/574 | ++ | +++ | + | |
| 575/576 | ++ | +++ | | |
| 577/578 | ++ | +++ | | |
| 579/580 | ++ | +++ | + | |
| 581/582 | ++ | +++ | ++ | |
| 583/584 | ++ | +++ | ++ | |
| 585/586 | ++ | +++ | | |
| 587/588 | ++ | ++ | | |
| 589/590 | ++ | +++ | | |
| 591/592 | ++ | +++ | | |
| 593/594 | ++ | ++ | | |
| 595/596 | ++ | +++ | | |
| 597/598 | ++ | +++ | ++ | |
| 599/600 | ++ | +++ | + | |
| 601/602 | ++ | +++ | | |
| 603/604 | ++ | +++ | | |
| 605/606 | ++ | +++ | | |
| 607/608 | ++ | +++ | | |
| 609/610 | ++ | +++ | | |
| 611/612 | ++ | +++ | | |
| 613/614 | ++ | +++ | | + |
| 615/616 | ++ | +++ | + | |
| 617/618 | ++ | ++ | | |
| 619/620 | ++ | +++ | | |
| 621/622 | ++ | +++ | | |
| 623/624 | ++ | +++ | + | |
| 625/626 | ++ | +++ | | |
| 627/628 | ++ | ++ | | |
| 629/630 | ++ | +++ | + | |
| 631/632 | ++ | +++ | | |
| 633/634 | ++ | +++ | | |
| 635/636 | ++ | +++ | | |
| 637/638 | ++ | +++ | ++++ | ++ |
| 639/640 | ++ | +++ | + | |
| 641/642 | ++ | +++ | + | |
| 643/644 | ++ | +++ | | |
| 645/646 | ++ | +++ | | |
| 647/648 | ++ | +++ | | |
| 649/650 | ++ | ++ | | |
| 651/652 | ++ | +++ | ++ | |
| 653/654 | ++ | +++ | +++ | |
| 655/656 | ++ | +++ | | |
| 657/658 | ++ | ++ | | |
| 659/660 | ++ | +++ | | |
| 661/662 | ++ | +++ | + | |
| 663/664 | + | ++ | | |
| 665/666 | ++ | +++ | | |
| 667/668 | ++ | +++ | + | |
| 669/670 | ++ | +++ | | |
| 671/672 | ++ | +++ | +++ | |
| 673/674 | ++ | +++ | | |
| 675/676 | ++ | +++ | | |
| 677/678 | ++ | +++ | | |
| 679/680 | ++ | +++ | | |
| 681/682 | ++ | +++ | | |
| 683/684 | ++ | +++ | | |
| 685/686 | ++ | +++ | | |
| 687/688 | ++ | +++ | | |
| 689/690 | ++ | +++ | | |
| 691/692 | ++ | +++ | | |
| 693/694 | ++ | +++ | | |
| 695/696 | ++ | +++ | | |
| 697/698 | ++ | +++ | | |
| 699/700 | ++ | +++ | | |
| 701/702 | ++ | +++ | | |
| 703/704 | ++ | +++ | +++ | + |
| 705/706 | ++ | +++ | | |
| 707/708 | ++ | +++ | | |
| 709/710 | ++ | ++ | | |
| 711/712 | ++ | +++ | | |
| 713/714 | ++ | +++ | | |
| 715/716 | ++ | ++ | | |
| 717/718 | ++ | +++ | + | |
| 719/720 | ++ | +++ | | |
| 721/722 | ++ | +++ | | |
| 723/724 | ++ | +++ | | |
| 725/726 | ++ | +++ | + | |
| 727/728 | ++ | +++ | + | + |
| 729/730 | ++ | +++ | | |
| 731/732 | ++ | +++ | + | ++ |
| 733/734 | ++ | +++ | + | |
| 735/736 | ++ | +++ | + | |
| 737/738 | ++ | +++ | ++ | |
| 739/740 | ++ | ++ | | |
| 741/742 | ++ | +++ | | |
| 743/744 | ++ | +++ | | |
| 745/746 | ++ | +++ | + | |
| 747/748 | ++ | +++ | ++ | |
| 749/750 | ++ | ++ | | |
| 751/752 | ++ | +++ | | |
| 753/754 | ++ | +++ | +++ | |

TIS reduction was measured as a reduction in the number of counts compared to the reference polypeptide of SEQ ID NO: 2 or SEQ ID NO: 12 and is defined as follows: “+” 1-10, “++” >10, “+++” >100, “++++” >200.
IHC reduction was measured as a reduction in the number of counts compared to the reference polypeptide of SEQ ID NO: 2 or SEQ ID NO: 12 and is defined as follows: “+” 1-2, “++” >2, “+++” >5, “++++” >20

### Example 9

### Characterization of GAA Variants in an Ex Vivo Immunogenicity Assessment

MHC II associated peptide proteomics (MAPPs) assays can be used to provide experimental evidence of HLA-binding epitopes from a protein antigen. The MAPPs assay combines the major steps of antigen uptake into differentiated antigen presenting cells, lysosomal processing, HLA binding, and presentation at the cell surface of an antigen presenting cell. The naturally processed, binding, and displayed peptides are then identified and quantified by liquid chromatography-mass spectrometry. Presentation of peptides at the cell surface by HLA-DR receptors to CD4+T helper cells is a required step in the activation cascade that includes T cell proliferation, differentiation, and ultimate production of antibodies from B cells. Therefore, decreased binding of a processed antigen to the groove of MHC II molecules and subsequent decreased presentation of an antigen is considered to reduce the immunogenicity risk potential of an antigen.

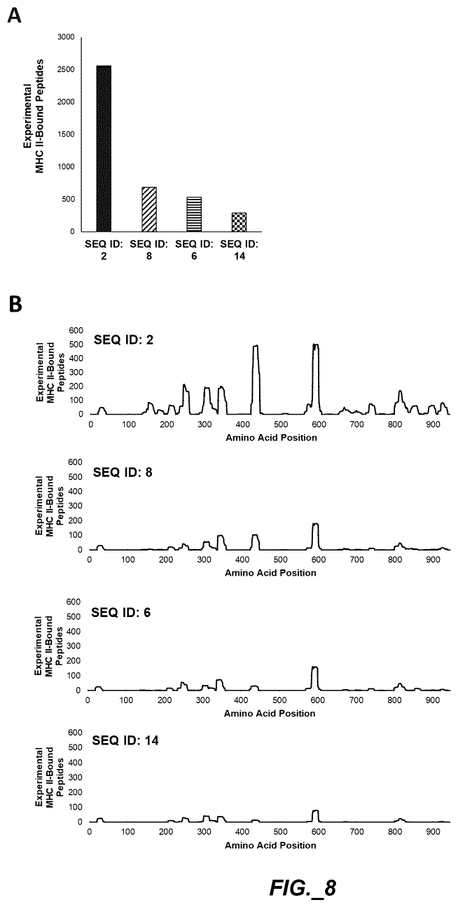

In this example, peripheral blood mononuclear cells (PBMCs) from healthy donor buffy coats were separated to isolate CD14+ monocytes which were subsequently differentiated into dendritic cells (DC) by methods known in the art. The DCs were then incubated with GAA variants and induced to a mature phenotype via addition of lipopolysaccharide. The HLA-DR binding peptides arising from processed antigens (GAA and variants) were captured by immunoprecipitation and eluted for LC-MS analysis. Identified binding peptides were aligned to the amino acid sequences of the GAA variants to enable comparison of processing and abundance of peptides originating from each GAA variant (Kropshofer and Spindeldreher (2005) in Antigen Presenting Cells:From Mechanisms to Drug Development, eds. Kropshofer and Vogt, Wiley-VCH, Weinheim, 159-98). By comparing the identified peptides, the GAA variants of SEQ ID NO: 6, SEQ ID NO: 8, and SEQ ID NO: 14 experimentally have significantly reduced processing and reduced frequency of peptide presentation as compared to the SEQ ID NO: 2 (see FIG. 8).

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

All publications, patents, patent applications and other documents cited in this application are hereby incorporated by reference in their entireties for all purposes to the same extent as if each individual publication, patent, patent application or other document were individually indicated to be incorporated by reference for all purposes.

What is claimed is:

1. An engineered acid alpha-glucosidase, or biologically active fragment thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99%, or more sequence identity to a reference sequence corresponding to residues 20 to 944 of SEQ ID NO: 12, or to a reference sequence corresponding to SEQ ID NO: 12, wherein the amino acid sequence comprises at least a substitution or amino acid residue 305V, 24A/C/D/F/G/H/I/K/M/N/P/S/T/V/Y, 28A/C/D/E/F/G/H/K/Q/T/V/W, 29A/C/D/E/F/G/H/I/K/M/N/P/R/W/Y, 39A/E/F/G/I/L/N/T, 50A/C/D/E/F/H/I/K/M/N/R/S/T/W/Y, 62D/H/I/K/M/N/P/Q/Y, 78A/C/D/F/G/H/I/K/L/M/N/Q/R/S/T/V/W/Y, 87A/G/H/I/K/L/MN/Q/R/S/T/V/W, 135C/D/E/F/G/H/I/K/L/N/R/Y, 266A/D/E/H/K/Q, 267H/L/T/V, 437A/H, 486C/D/F/G/H/I/K/L/M/N/Q/R/S/V/W/Y, 522A/C/D/F/G/H/I/K/L/M/N/P/Q/R/S/T/W/Y, 569A/C/D/E/G/K/M/N/P/R/W, 670A/D/G/H/K/M/Y, 692A/D/E/H/K/L/M/N/T/W, 711D/E/I/K/M/N/Q/S/T/V/Y, 736F/L, 750E/K/L/Q/R, 812A/D/G/S, 830D/E/F/G/H/L/M/N/S/T/W/Y, 842A/C/D/F/H/K/L/M/N/Q/R/T/W, 871A/C/D/F/H/I/M/N/Q/T/V/W/Y, 883A/F/Q, 894A/D/E/H/I/K/L/M/N/S/T/V/W/Y, 913F/I/K/M/N/S, or 932C/D/E/G/H/K/L/M/N/P/Q/R/W/Y, or combinations thereof, wherein the amino acid positions are relative to the reference sequence corresponding to residues 20 to 944 of SEQ ID NO: 12 or 2, or to a reference sequence corresponding to SEQ ID NO: 12 or 2.

2. An engineered acid alpha-glucosidase, or biologically active fragment thereof, comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-944 of an even numbered SEQ ID NO. of SEQ ID NOs: 14-754, or to an even numbered SEQ ID NO. of SEQ ID NOs: 14-754, wherein the amino acid sequence comprises at least a substitution or amino acid residue 305V, 24A/C/D/F/G/H/I/K/M/N/P/S/T/V/Y, 28A/C/D/E/F/G/H/K/Q/T/V/W, 29A/C/D/E/F/G/H/I/K/M/N/P/R/W/Y, 39A/E/F/G/I/L/N/T, 50A/C/D/E/F/H/I/K/M/N/R/S/T/W/Y, 62D/H/I/K/M/N/P/Q/Y, 78A/C/D/F/G/H/I/K/L/M/N/Q/R/S/T/V/W/Y, 87A/G/H/I/K/L/MN/Q/R/S/T/V/W, 135C/D/E/F/G/H/I/K/L/N/R/Y, 266A/D/E/H/K/Q, 267H/L/T/V, 437A/H, 486C/D/F/G/H/I/K/L/M/N/Q/R/S/V/W/Y, 522A/C/D/F/G/H/I/K/L/M/N/P/Q/R/S/T/W/Y, 569A/C/D/E/G/K/M/N/P/R/W, 670A/D/G/H/K/M/Y, 692A/D/E/H/K/L/M/N/T/W, 711D/E/I/K/M/N/Q/S/T/V/Y, 736F/L, 750E/K/L/Q/R, 812A/D/G/S, 830D/E/F/G/H/L/M/N/S/T/W/Y, 842A/C/D/F/H/K/L/M/N/Q/R/T/W, 871A/C/D/F/H/I/M/N/Q/T/V/W/Y, 883A/F/Q, 894A/D/E/H/I/K/L/M/N/S/T/V/W/Y, 913F/I/K/M/N/S, or 932C/D/E/G/H/K/L/M/N/P/Q/R/W/Y, or combinations thereof, wherein the amino acid positions are relative to the reference sequence corresponding to residues 20 to 944 of SEQ ID NO: 2, or to a reference sequence corresponding to SEQ ID NO: 2.

3. The engineered acid alpha-glucosidase of claim 1, wherein the amino acid sequence of the engineered acid alpha-glucosidase comprises residues 20 to 944 of an even-numbered SEQ ID NO. of SEQ ID NOs: 14-754, or comprises an even-numbered SEQ ID NO. of SEQ ID NOs: 14-754.

4. The engineered acid alpha-glucosidase of claim 1, wherein the amino acid sequence of the engineered acid alpha-glucosidase comprises residues 20 to 944 of SEQ ID NO: 14, 114, 126, 170, 250, 252, 394, 472, 488, or 506, or comprises SEQ ID NO: 14, 114, 126, 170, 250, 252, 394, 472, 488, or 506.

5. The engineered acid alpha-glucosidase of claim 1, wherein the engineered acid alpha-glucosidase exhibits at least one improved property selected from: i) enhanced catalytic activity; ii) increased tolerance to pH 7; iii) increased tolerance to pH 4.4; iv) increased stability in lysosomes; v) increased expression in cells; vi) increased uptake into cells; vii) increased enzymatic activity in cell lysates; viii) increased stability in plasma/serum; and ix) reduced immunogenicity; or a combination of any of i), ii), iii), iv), v), vi), vii), viii), and ix), as compared to a reference acid alpha-glucosidase having a sequence corresponding to residues 20 to 944 of SEQ ID NO: 2 or 12, or a sequence corresponding to SEQ ID NO: 2 or 12.

6. The engineered acid alpha-glucosidase of claim 5, wherein the engineered acid alpha-glucosidase exhibits reduced immunogenicity as compared to the reference acid alpha-glucosidase having a sequence corresponding to residues 20 to 944 of SEQ ID NO: 2 or 12, or a sequence corresponding to SEQ ID NO: 2 or 12.

7. The engineered acid alpha-glucosidase of claim 6, wherein the engineered acid alpha-glucosidase exhibits (a) a reduction in Total Immunogenic Score (TIS) of greater than 10 as compared to the reference acid alpha-glucosidase of SEQ ID NO: 2; (b) a reduction in Immunogenic Hit Count (IHC) of greater than 2 as compared to the reference acid alpha-glucosidase of SEQ ID NO: 2; (c) a reduction in Total Immunogenic Score (TIS) of greater than 10 as compared to the reference acid alpha-glucosidase of SEQ ID NO: 12; and/or (d) a reduction in Immunogenic Hit Count (IHC) of greater than 2 as compared to the reference acid alpha-glucosidase of SEQ ID NO: 12.

8. The engineered acid alpha-glucosidase of claim 1, comprising a pre-pro-peptide of the engineered acid alpha-glucosidase.

9. The engineered acid alpha-glucosidase of a claim 8, wherein the pre-pro-peptide of the engineered acid alpha-glucosidase comprises a eukaryotic or synthetic signal peptide sequence.

10. The engineered acid alpha-glucosidase of claim 9, wherein the signal peptide comprises a mouse or human signal peptide sequence.

11. The engineered acid alpha-glucosidase of claim 1, comprising a pro-peptide of the engineered acid alpha-glucosidase.

12. The engineered acid alpha-glucosidase of claim 1, wherein the engineered acid alpha-glucosidase is purified.

13. A pharmaceutical composition comprising an engineered acid alpha-glucosidase of claim 1.

14. The pharmaceutical composition of claim 13, further comprising a pharmaceutically acceptable carrier and/or excipient.

15. The pharmaceutical composition of claim 13, wherein the composition is suitable for parenteral injection or infusion to a human.

16. A recombinant polynucleotide comprising a polynucleotide sequence encoding an engineered acid alpha-glucosidase of claim 1.

17. The recombinant polynucleotide of claim 16, comprising a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference polynucleotide sequence corresponding to nucleotide residues 58-2832 of an odd-numbered SEQ ID NO. of SEQ ID NOs: 13-753, or to a reference polynucleotide sequence corresponding to an odd-numbered SEQ ID NO. of SEQ ID NOs: 13-753, wherein the polynucleotide encodes an acid alpha-glucosidase.

18. The recombinant polynucleotide of claim 16, comprising a polynucleotide sequence codon-optimized for expression of the encoded engineered acid alpha-glucosidase.

19. The recombinant polynucleotide of claim 16, comprising a polynucleotide sequence comprising nucleotide residues 58-2832 of an odd-numbered SEQ ID NO. of SEQ ID NOs: 13-753, or a polynucleotide sequence comprising an odd-numbered SEQ ID NO. of SEQ ID NOs: 13-753.

20. The recombinant polynucleotide of claim 16, comprising a polynucleotide sequence comprising nucleotide residues 58-2832 of SEQ ID NO: 13, 113, 125, 169, 249, 251, 393, 471, 487, or 505, or a polynucleotide sequence comprising SEQ ID NO: 13, 113, 125, 169, 249, 251, 393, 471, 487, or 505.

21. An expression vector comprising the recombinant polynucleotide of claim 16.

22. The expression vector of claim 21, wherein the recombinant polynucleotide is operably linked to a control sequence.

23. The expression vector of claim 22, wherein the control sequence comprises a promoter.

24. The expression vector of claim 23, wherein the promoter is a heterologous promoter.

25. A host cell comprising the expression vector of claim 21.

26. The host cell of claim 25, wherein the host cell is a eukaryotic cell or prokaryotic cell.

27. The host cell of claim 25, wherein the host cell is a mammalian cell.

28. The host cell of claim 27, wherein the mammalian cell is a human cell.

29. The host cell of claim 28, wherein the human cell is from a patient having a deficiency in acid alpha-glucosidase activity.

30. A method of producing an engineered acid alpha-glucosidase variant, comprising culturing the host cell of claim 25 under suitable conditions such that the acid alpha-glucosidase encoded by the recombinant polynucleotide is produced.

31. The method of claim 30, further comprising the step of recovering the acid alpha-glucosidase.

32. The method of claim 30, further comprising purifying the acid alpha-glucosidase.

33. A method for treating and/or preventing symptoms of a deficiency in acid alpha-glucosidase in a subject, comprising administering to a subject in need thereof an effective amount of an engineered acid alpha-glucosidase of claim 1.

34. The method of claim 33, wherein the deficiency in acid alpha-glucosidase is Pompe disease.

35. The method of claim 34, wherein the subject is an infant or child.

36. The method of claim 34, wherein the subject is an adult or young adult.
